SEQUENCE LISTING (28 sequences)

SEQ ID NO: 1 - protein, 108 aa, Rattus norvegicus
DIQMTQSPTSLSASLGETVSIECRASEDIYNNLAWYQQKSGKSPQLLIYYASTLQDGVPSRFSGSGSGTQFSLKISGMQPEDEGLYYCLQGSKYPRTFGGGTKLELKR

SEQ ID NO: 2 - DNA, 324 bp, Rattus norvegicus
gacatccagatgacacagtctccaacttccctgtctgcatctctgggagagactgtctccattgaatgtcgagcaagtgaggacatttacaataatttagcgtggtatcagcagaagtcagggaaatctcctcagcttttgatctattatgcaagtaccttgcaagatggggtcccatcacggttcagtggcagtggatctggcacacagttttctctcaagatcagtggcatgcaacctgaagatgaagggctttattactgtctacaaggttccaagtatcctcggacgttcggtggaggcaccaagctggaattgaaacgg

SEQ ID NO: 3 - protein, 151 aa, Rattus norvegicus
EEQLVESGGGLVQPGGSLKLSCLASGFTLSNYGMNWIRQAPGKGLEWVASISSSSSYFYYADTVKGRFTISREDAKNTLNLQMTSLRSEDTALYYCARGITIAAGFTMDAWGQGTSVTVSSAETTAPSVYPLAPGAAAQTNSMVTLGCLVK

SEQ ID NO: 4 - DNA, 363 bp, Rattus norvegicus
gaggagcaactggtggagtctggaggaggcttagtgcagcctggaggttccctgaaactctcctgcttagcctctggattcactctcagtaactatggaatgaactggattcgccaggctccagggaaggggctggagtgggttgcatctattagtagtagtagcagttacttctactatgcagacacagtgaagggccgattcaccatctccagagaggatgccaagaacaccctgaacctgcaaatgaccagtctgaggtctgaagacactgccttgtattactgtgcaagagggattactatagcagctggctttactatggatgcctggggtcaaggaacttcagtcactgtctcctca

SEQ ID NO: 5 - protein, 219 aa, Rattus norvegicus
DIVMTQSPSSLAVSAGETVTINCKSSQSLLSSRNQKNYLAWYQQKPGQSPKLLIYLASTRESGVPDRFIGSGSGTDFTLTITSVQAEDLADYYCQQHYNYYTFGAGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 6 - DNA, 339 bp, Rattus norvegicus
gacattgtgatgacccaatctccatcctccctggctgtgtcagcaggagagacggtcactataaactgcaagtccagtcagagtcttttatccagtagaaaccaaaagaactacttggcctggtaccagcagaaaccagggcagtctcctaaactgctgatctacttggcatccactagggaatctggtgtccctgatcgcttcataggcagtggatctgggacagacttcactctgaccatcaccagtgtgcaggctgaagatctggcagattattactgtcagcagcattacaactattacacgtttggagctgggaccaagctggaactgaaacgg

SEQ ID NO: 7 - protein, 150 aa, Rattus norvegicus
QVQLKESGPGLVQPSQTLSLTCTVSGFSLTSHAVNWVRQPPGKGLEWIGAIWSGGTTDYNSGFRSRLSISRDTSKSQVLLKMNSLQTEDTAMYFCARGYDGYYYYAMDVWGQGTSVTVSSAETTAPSVYPLAPGAAAQTNSMVTLGCLVK

SEQ ID NO: 8 - DNA, 360 bp, Rattus norvegicus
caggtgcagctgaaggagtcaggacctggtctggtgcagccctcacagaccctgtccctcacctgcactgtctctggattctcattaaccagccatgctgtaaactgggttcgccagcctccaggaaagggtctggagtggattggagcaatatggagtggtggaaccacagattataattcaggtttcagatcccgactgagcatcagcagggacacctccaagagccaagttctcttaaaaatgaacagtctgcaaactgaagacacagccatgtacttctgtgccagagggtatgatggttattattattatgctatggatgtctggggtcaaggaacttcagtcactgtctcctca

SEQ ID NO: 9 - protein, 128 aa, Rattus norvegicus
MAIPTQLLGLLLLWITDAICDIQMTQSPTSLSASLGETVSIECRASEDIYNNLAWYQQKSGKSPQLLIYYASTLQDGVPSRFSGSGSGTQFSLKISGMQPEDEGLYYCLQGSKYPRTFGGGTKLELKR

SEQ ID NO: 10 - DNA, 384 bp, Rattus norvegicus
atggctattcccactcagctcttggggttgttgttactgtggattacagatgccatatgtgacatccagatgacacagtctccaacttccctgtctgcatctctgggagagactgtctccattgaatgtcgagcaagtgaggacatttacaataatttagcgtggtatcagcagaagtcagggaaatctcctcagcttttgatctattatgcaagtaccttgcaagatggggtcccatcacggttcagtggcagtggatctggcacacagttttctctcaagatcagtggcatgcaacctgaagatgaagggctttattactgtctacaaggttccaagtatcctcggacgttcggtggaggcaccaagctggaattgaaacgg

SEQ ID NO: 11 - protein, 170 aa, Rattus norvegicus
MNFSNTLVFLLFLLKGILCEEQLVESGGGLVQPGGSLKLSCLASGFTLSNYGMNWIRQAPGKGLEWVASISSSSSYFYYADTVKGRFTISREDAKNTLNLQMTSLRSEDTALYYCARGITIAAGFTMDAWGQGTSVTVSSAETTAPSVYPLAPGAAAQTNSMVTLGCLVK

SEQ ID NO: 12 - DNA, 420 bp, Rattus norvegicus
atgaatttcagcaacaccttggttttccttttgtttcttttaaaaggtatcctgtgtgaggagcaactggtggagtctggaggaggcttagtgcagcctggaggttccctgaaactctcctgcttagcctctggattcactctcagtaactatggaatgaactggattcgccaggctccagggaaggggctggagtgggttgcatctattagtagtagtagcagttacttctactatgcagacacagtgaagggccgattcaccatctccagagaggatgccaagaacaccctgaacctgcaaatgaccagtctgaggtctgaagacactgccttgtattactgtgcaagagggattactatagcagctggctttactatggatgcctggggtcaaggaacttcagtcactgtctcctca

SEQ ID NO: 13 - protein, 239 aa, Rattus norvegicus
MESQTQVLMSLLLWISGTCGDIVMTQSPSSLAVSAGETVTINCKSSQSLLSSRNQKNYLAWYQQKPGQSPKLLIYLASTRESGVPDRFIGSGSGTDFTLTITSVQAEDLADYYCQQHYNYYTFGAGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 14 - DNA, 399 bp, Rattus norvegicus
atggaatcacagacccaggtcctcatgtccctgctgctctggatttctggtacctgtggggacattgtgatgacccaatctccatcctccctggctgtgtcagcaggagagacggtcactataaactgcaagtccagtcagagtcttttatccagtagaaaccaaaagaactacttggcctggtaccagcagaaaccagggcagtctcctaaactgctgatctacttggcatccactagggaatctggtgtccctgatcgcttcataggcagtggatctgggacagacttcactctgaccatcaccagtgtgcaggctgaagatctggcagattattactgtcagcagcattacaactattacacgtttggagctgggaccaagctggaactgaaacgg

SEQ ID NO: 15 - protein, 169 aa, Rattus norvegicus
MAVLVLLLCLVTFPSCVLSQVQLKESGPGLVQPSQTLSLTCTVSGFSLTSHAVNWVRQPPGKGLEWIGAIWSGGTTDYNSGFRSRLSISRDTSKSQVLLKMNSLQTEDTAMYFCARGYDGYYYYAMDVWGQGTSVTVSSAETTAPSVYPLAPGAAAQTNSMVTLGCLVK

SEQ ID NO: 16 - DNA, 417 bp, Rattus norvegicus
atggctgtcctggtgctgttgctctgcctggtgacatttccaagctgtgtcctgtcccaggtgcagctgaaggagtcaggacctggtctggtgcagccctcacagaccctgtccctcacctgcactgtctctggattctcattaaccagccatgctgtaaactgggttcgccagcctccaggaaagggtctggagtggattggagcaatatggagtggtggaaccacagattataattcaggtttcagatcccgactgagcatcagcagggacacctccaagagccaagttctcttaaaaatgaacagtctgcaaactgaagacacagccatgtacttctgtgccagagggtatgatggttattattattatgctatggatgtctggggtcaaggaacttcagtcactgtctcctca

SEQ ID NO: 17 - DNA, 43 bp, Artificial
  primer
cattctggatggggctgtctaataggaaccccagctacccatg

SEQ ID NO: 18 - DNA, 43 bp, Artificial
  primer
catgggtagctggggttcctattagacagccccatccagaatg

SEQ ID NO: 19 - DNA, 42 bp, Artificial
  primer
cctttgatgccccacttatttaatgtccgaggcgctgtctcc

SEQ ID NO: 20 - DNA, 42 bp, Artificial
  primer
ggagacagcgcctcggacattaaataagtggggcatcaaagg

SEQ ID NO: 21 - DNA, 46 bp, Artificial
  primer
gatgccccacttatttagagtcaatggcgctgtctcccagacatac

SEQ ID NO: 22 - DNA, 46 bp, Artificial
  primer
gtatgtctgggagacagcgccattgactctaaataagtggggcatc

SEQ ID NO: 23 - DNA, 47 bp, Artificial
  primer
caggtacctgtgcatatatacaaaatggagctgtttatgcggaaaac

SEQ ID NO: 24 - DNA, 47 bp, Artificial
  primer
gttttccgcataaacagctccattttgtatatatgcacaggtacctg

SEQ ID NO: 25 - RNA, 19 nt, Artificial
  siRNA
ggauuaguagugaccauua

SEQ ID NO: 26 - RNA, 19 nt, Artificial
  siRNA
ugucugaccuccuaacaca

SEQ ID NO: 27 - RNA, 19 nt, Artificial
  siRNA
cagaagaaggcaaaccuaa

SEQ ID NO: 28 - RNA, 19 nt, Artificial
  siRNA
gagaagugcuugucuuugg

(19) United States
(12) Patent Application Publication
(10) Pub. No.: US 2009/0311271 A1
(43) Pub. Date: Dec. 17, 2009

(54) Methods and Compositions for Selective Inhibition of Ligand Binding to the Lectin-Like Receptor for Oxidized Low Density Lipoprotein (LOX-1)

(75) Inventors: Douglas Carl Harnish, Pennsburg, PA (US); Songwen Zhang, Lansdale, PA (US); Macy Xiaohang Jin, Lexington, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/473,812

(22) Filed: May 28, 2009

Related U.S. Application Data
(60) Provisional application No. 61/075,084, filed on Jun. 24, 2008.
(60) Provisional application No. 61/057,005, filed on May 29, 2008.

(51) Int. Cl. A61K 39/395; C12N 5/06; C12N 1/20; C07K 16/18

(52) U.S. Cl. 424/172.1; 435/375; 435/252.1; 530/387.1

(57) ABSTRACT

The present invention provides methods of selectively inhibiting the binding of one ligand for LOX-1, but not one other ligand for LOX-1. Moreover, the invention relates to the identification of binding partners that act in a selective manner to inhibit the binding of one ligand to LOX-1, but not one other ligand for LOX-1, and methods of identifying such binding partners. Pharmaceutical compositions comprising the binding partners for LOX-1, in particular, anti-LOX-1 antibodies or fragments thereof, are also provided in the present invention.

### CROSS-REFERENCE TO RELATED APPLICATIONS

This application claims priority under 35 U.S.C. §119(e) to U.S. provisional application Ser. No. 61/057,005, filed on May 29, 2008, and to U.S. provisional application Ser. No. 61/075,084, filed on Jun. 24, 2008, both of which are incorporated herein by reference in their entireties.

### FIELD OF THE INVENTION

The present invention relates generally to methods and compositions for selectively inhibiting binding of at least one ligand to LOX-1, without inhibiting the binding of one other ligand to LOX-1. The present invention relates more particularly to the identification of binding partners that show selectivity for blocking the binding of at least one ligand, without inhibiting the binding of at least a second ligand, to LOX-1. The compositions comprise at least one binding partner, which demonstrates selectivity for blocking the binding of one ligand, but not a second ligand for LOX-1.

### BACKGROUND OF THE INVENTION

Atherosclerosis is a chronic inflammatory disease that results from hyperlipidemia, as well as, a complex interplay of a variety of environmental, metabolic and genetic risk factors. The oxidation of low density lipoprotein (LDL) plays a central, if not obligatory role, in the atherogenic process. Inflammation is a key component of atherosclerosis (V Willerson J T, Ridker P M. Circulation. 2004; 109:II2-10).

The lectin-like oxidized low-density lipoprotein (oxLDL) receptor-1 (LOX-1) is a 50 kDa type II membrane protein that structurally belongs to the C-type lectin family with a short intracellular N-terminal hydrophilic and a long extracellular C-terminal hydrophilic domain separated by a hydrophobic domain of 26 amino acids. The human LOX-1 gene is encoded by 6 exons spanning about 15 kb in the short arm of chromosome 12 (Aoyama, T. et al. Biochem. J. 1999; 339 (Pt 1): 177-184). LOX-1 is the primary oxLDL receptor in endothelial cells (Sawamura T, Kume N, Aoyama T, Moriwaki H, Hoshikawa H, Aiba Y, Tanaka T, Miwa S, Katsura Y, Kita T, Masaki T. Nature. 1997; 386:73-77) and as such, mediates most of the toxic effects of ox-LDL. LOX-1 is also present on other cell types, including macrophages, monocytes, dendritic cells, vascular smooth muscle cells (SMC), chondrocytes and cardiac myocytes. LOX-1 is highly expressed in vivo in large arteries (aortic, carotid, thoracic, coronary arteries and veins), which are the predilection sites of atherosclerosis (Sawamura, T. et al. Nature 1997; 386:73-77; and Shi, X. et al. J. Cell Sci, 2001; 114:1273-1282). Furthermore, LOX-1 is found in atherosclerotic lesions in humans and in experimental animal models. Moreover, macrophages and smooth muscle cells in the intima of advanced atherosclerotic plaques are positive for LOX-1 suggesting that LOX-1 may play a role in the early stages of atherosclerosis (Kataoka, H. et al. Circulation, 1999; 99:3310-3117). LOX-1 shows multiple ligand binding activity and several studies indicate that LOX-1 can be regulated at both the transcriptional and translational levels by inflammatory cytokines, oxidative stress, chemicals, as well as, pathological conditions.

In addition to its role in atherosclerosis, LOX-1 is also associated with several other cardiovascular conditions including acute myocardial infarction, hypertension and coronary heart disease (Chen, X. et al. Chinese Medical J., 2007, 120 (5): 421-426; Ohmori R, Momiyama Y, Nagano M, Taniguchi H, Egashira T, Yonemura A, Nakamura H, Kondo K, Ohsuzu F. Clinical Cardiology. 2004; 27:641-644; Tatsuguchi M, Furutani M, Hinagata J, Tanaka T, Furutani Y, Imamura S, Kawana M, Masaki T, Kasanuki H, Sawamura T, Matsuoka R. Biochemical & Biophysical Research Communications. 2003; 303:247-250; Puccetti L, Pasqui A L, Bruni F, Pastorelli M, Clani F, Palazzuoli A, Pontani A, Ghezzi A, Auteri A. International Journal of Cardiology. 2007; 1119:41-47; Mingyi, C. et al. Pharmacol. Ther. 2002; 95:89-100). Activation of cell surface expressed LOX-1 on endothelial cells is tethered to downstream inflammatory signaling pathways including NF-κB and MAPK resulting in inflammatory gene expression, superoxide generation, eNOS deficiency, and increased monocyte adhesion to endothelial cells (Mehta J L, Chen J, Hermonat P L, Romeo F, Novelli G. Cardiovasc Res. 2006; 69:36-45) similar to that observed for C reactive protein (CRP). As a scavenger receptor, LOX-1 is capable of interacting with a variety of structurally and functionally distinct ligands including oxLDL, platelets, aged red blood cells, apoptotic cells, advanced glycation end products (AGEs), HSP70, bacteria, and phosphatidylserine (Chen X P, Zhang T T, Du G H. Cardiovascular Drug Reviews. 2007; 25: 146-161.).

A relatively non-specific charge-to-charge interaction is believed to be the common mechanism by which LOX-1 interacts with these diverse ligands. The crystal structure of LOX-1 reveals that a number of positively charged arginine residues on the surface of the LOX-1 homodimer forms a “basic-spine” structure that mediates the binding to negatively charged ligands such as oxLDL (Ishigaki T, Ohki I, Oyama T, Machida S, Morikawa K, Tate S. Acta Crystallograph Sect F Struct Biol Cryst Commun. 2005; 61:524-527; Ohki I, Ishigaki T, Oyama T, Matsunaga S, Xie Q, Ohnishi-Kameyama M, Murata T, Tsuchiya D, Machida S, Morikawa K, Tate S. Structure. 2005; 13:905-917; Park H, Adsit F G, Boyington J C. J Biol. Chem. 2005; 280: 13593-13599).

Prevention of binding of certain ligands to LOX-1 may result in inhibition of many of the various deleterious effects caused by ligand receptor interaction. However, it may be beneficial to retain certain functions associated with ligand binding to LOX-1, in particular, certain scavenging functions associated with receptor activation. Accordingly, there remains a need in the art for improved methods for selectively inhibiting the interaction of certain ligands with the LOX-1 receptor, while retaining the binding of other ligands to the LOX-1 receptor. The present invention addresses these needs and provides a method and compositions for such selective binding of ligands to LOX-1.

### SUMMARY OF THE INVENTION

The present invention provides methods and compositions for selectively inhibiting binding of one ligand to a lectin-like oxidized low-density lipoprotein receptor (LOX-1), without inhibiting binding of another ligand to LOX-1. In particular, the present invention demonstrates that C-reactive protein (CRP) can directly interact with LOX-1, yet the mechanism for binding of CRP to LOX-1 appears to be distinct from the interaction of other ligands with LOX-1, such as, but not limited to, the interaction of oxidized low density lipoprotein (oxLDL) with LOX-1. Accordingly, the results presented herein demonstrate that LOX-1 is a novel receptor for CRP and that some of the pathologic activities associated with CRP may be mediated by LOX-1 downstream signaling. Furthermore, the studies presented herein demonstrate that it is possible to generate a binding partner, such as, but not limited to, an antibody, to LOX-1, which interferes with binding of one ligand to LOX-1, but does not interfere with binding of another ligand to LOX-1. Based on these findings, it is possible that certain detrimental effects associated with particular ligand binding to LOX-1 may be inhibited, while maintaining certain beneficial effects associated with the binding of other ligands to LOX-1.

Accordingly, one embodiment of the invention provides a method of selectively inhibiting binding of a ligand to a lectin-like oxidized low-density lipoprotein receptor (LOX-1), comprising contacting the LOX-1 with a binding partner that inhibits binding of at least one ligand to LOX-1, but not one other ligand for LOX-1.

In one embodiment, the methods provide for selectively inhibiting binding of a ligand to LOX-1 that is expressed on a cell. In one embodiment, the cell is selected from the group consisting of an endothelial cell, a macrophage, a monocyte, a dendritic cell, a vascular smooth muscle cell (SMC) a chondrocyte, a platelet, an intestinal cell and a cardiac myocyte.

In one embodiment, the methods provide for selective inhibition of binding of a ligand to LOX-1 using a binding partner that is selected from the group consisting of a polypeptide, an antibody and a small molecule. The small molecule may be a synthetic, semi-synthetic, or naturally derived organic compound, or an organic compound coupled with an inorganic compound that has a molecular weight of less than about 3,000 daltons. In one embodiment, the binding partner is an antibody. The antibody may be a monoclonal antibody, a polyclonal antibody, a chimeric antibody, a single chain antibody or a fragment of any one of the monoclonal, polyclonal, chimeric or single chain antibodies. In one embodiment, the antibody is selected from the group consisting of a mouse antibody, a rat antibody, a goat antibody, a sheep antibody, a rabbit antibody, a porcine antibody, a horse antibody, a human antibody and a humanized antibody.

In one embodiment, certain ligands that may be selectively inhibited using the methods described herein comprise oxidized low density lipoprotein (ox-LDL) and C reactive protein (CRP).

In one embodiment, the methods described herein provide a binding partner that inhibits the binding of oxidized LDL with LOX-1, but does not inhibit the binding of C reactive protein with LOX-1.

In one embodiment, the methods described herein utilize a binding partner, which is an antibody such as, but not limited to, a rat anti-mouse LOX-1 antibody.

In one embodiment, the methods described herein utilize a binding partner that is a rat anti-mouse LOX-1 antibody comprising a variable light (V_{L}) chain amino acid sequence comprising the amino acid sequence of SEQ ID NO: 1 and a variable heavy (V_{H}) chain amino acid sequence comprising the amino acid sequence of SEQ ID NO: 3. In one embodiment, the V_{L} chain is encoded by a nucleic acid sequence comprising the nucleic acid sequence of SEQ ID NO: 2 and wherein the V_{H} chain is encoded by a nucleic acid sequence comprising the nucleic acid sequence of SEQ ID NO: 4.

In one embodiment, the methods described herein utilize a binding partner that is a rat anti-mouse LOX-1 antibody comprising a variable light (V_{L}) chain amino acid sequence comprising the amino acid sequence of SEQ ID NO: 5 and a variable heavy (V_{H}) chain amino acid sequence comprising the amino acid sequence of SEQ ID NO: 7. In one embodiment, the V_{L} chain is encoded by a nucleic acid sequence comprising the nucleic acid sequence of SEQ ID NO: 6 and wherein the V_{H} chain is encoded by a nucleic acid sequence comprising the nucleic acid sequence of SEQ ID NO: 8.

In one embodiment, the methods described herein utilize a ligand that binds to LOX-1 that is selected from the group consisting of a modified lipoprotein, an anionic phospholipid, a cellular ligand, a bile salt-dependent lipase and C-reactive protein. In one embodiment, the modified lipoprotein is selected from the group consisting of oxidized low density lipoprotein (ox-LDL), acetylated low density lipoprotein (Ac-LDL), and advanced glycation end-products (AGEs). In one embodiment, the anionic phospholipids is phosphatidylserine or phosphatidylinositol. In one embodiment, the cellular ligand is selected from the group consisting of apoptotic cells, aged cells, activated platelets and bacterial cells.

In one embodiment, the methods of the present invention, described above, result in elimination of at least one detrimental biological effect associated with ligand binding to LOX-1, but retains one or more other non-detrimental biological effects associated with ligand binding to LOX-1.

One embodiment of the invention provides an isolated or purified binding partner that interacts with, or binds to LOX-1, wherein the binding partner is characterized by its ability to inhibit the binding of at least one ligand to LOX-1, but not one other ligand for LOX-1.

In one embodiment, the binding partner is selected from the group consisting of a polypeptide, an antibody and a small molecule. The small molecule may be a synthetic, semi-synthetic, or naturally derived organic compound, or an organic compound coupled with an inorganic compound that has a molecular weight of less than about 3,000 daltons. In one embodiment, the binding partner is an antibody, which may be a monoclonal antibody, a polyclonal antibody, a chimeric antibody, a single chain antibody, or a fragment of any one of the monoclonal, polyclonal, chimeric or a single chain antibody. In one embodiment, the binding partner is an antibody selected from the group consisting of a mouse antibody, a rat antibody, a goat antibody, a sheep antibody, a rabbit antibody, a porcine antibody, a horse antibody, a human antibody and a humanized antibody.

In one embodiment, the binding partner is a rat anti-mouse LOX-1 antibody.

In one embodiment, the binding partner is a rat anti-mouse LOX-1 antibody comprising a variable light (V_{L}) chain amino acid sequence comprising the amino acid sequence of SEQ ID NO: 1 and a variable heavy (V_{H}) chain amino acid sequence comprising the amino acid sequence of SEQ ID NO: 3. In one embodiment, the binding partner is a rat anti-mouse LOX-1 antibody comprising a V_{L} chain encoded by a nucleic acid sequence comprising the nucleic acid sequence of SEQ ID NO: 2 and a V_{H} chain encoded by a nucleic acid sequence comprising the nucleic acid sequence of SEQ ID NO: 4.

In one embodiment, the binding partner is a rat anti-mouse LOX-1 antibody comprising a variable light (V_{L}) chain amino acid sequence comprising the amino acid sequence of SEQ ID NO: 5 and a variable heavy (V_{H}) chain amino acid sequence comprising the amino acid sequence of SEQ ID NO: 7. In one embodiment, the binding partner is a rat anti-mouse LOX-1 antibody comprising a V_{L} chain encoded by a nucleic acid sequence comprising the nucleic acid sequence of SEQ ID NO: 6 and a V_{H} chain is encoded by a nucleic acid sequence comprising the nucleic acid sequence of SEQ ID NO: 8.

In one embodiment, the binding partner, as described above, inhibits the binding of oxidized LDL with LOX-1, but does not inhibit the binding of C reactive protein with LOX-1.

One embodiment of the invention provides a pharmaceutical composition comprising a therapeutically effective amount of at least one of the binding partners, as described herein.

One embodiment of the invention provides a method of treating a mammal suffering from a disease or condition associated with elevated levels of LOX-1 or a LOX-1 ligand, comprising administering an isolated or purified LOX-1 binding partner, as described above and a pharmaceutically acceptable carrier.

In one embodiment, the mammal in need of such treatment is a human or non-human mammal.

In one embodiment, the disease or condition associated with elevated levels of a LOX-1 ligand, for which treatment is desired is selected from the group consisting of atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation.

In one embodiment, the LOX-1 binding partner is administered to a mammal enterally or parenterally.

In one embodiment, the LOX-1 binding partner is administered intravenously, intramuscularly, subcutaneously, sublingually, or bucally.

One embodiment of the invention provides a method of screening for an inhibitor of C reactive protein (CRP) binding to LOX-1, the method comprising:
a) combining LOX-1 with CRP and a candidate inhibitor; and
b) determining whether or not the candidate inhibitor interferes with LOX-1 binding to CRP;
   wherein a candidate inhibitor that interferes with the binding of LOX-1 to CRP is identified as an inhibitor of CRP binding to LOX-1.

In one embodiment, the method of screening is performed in a cell-free system.

In one embodiment, the method of screening is performed in a cell-based system.

In one embodiment, the method of screening is performed in vitro.

In one embodiment, the method of screening provides for a candidate inhibitor that is selected from the group consisting of a polypeptide, an antibody or a small molecule.

In one embodiment, the method of screening provides for CRP coupled to a detectable label.

In one embodiment, the method of screening provides for a candidate inhibitor to be coupled to a detectable label.

In one embodiment, the method of screening provides for the LOX-1, or the CRP attached to a solid support.

In one embodiment, the method of screening provides for the candidate inhibitor to be determined by an assay selected from the group consisting of an Enzyme Linked Immunoassay (ELISA), fluorescence activated cell sorting, fluorescent resonance energy transfer (FRET), Alphascreen, fluorescence polarization (FP) or surface plasmon resonance (SPR).

One embodiment of the invention provides a method of screening for a selective LOX-1 inhibitor, wherein the inhibitor selectively inhibits the binding of one ligand, but not one other ligand to LOX-1, the method comprising:
a) combining LOX1 with the first ligand and a candidate inhibitor;
b) determining whether or not the candidate inhibitor interferes with LOX1 binding to the first ligand;
c) combining LOX1 with the candidate inhibitor and the second ligand;
d) determining whether or not the candidate inhibitor interferes with LOX1 binding to the second ligand;

wherein a candidate inhibitor that interferes with binding of LOX1 to the first ligand but does not interfere with binding of LOX1 to the second ligand is selective for binding of LOX1 to the first ligand.

In one embodiment, the method of screening is performed in a cell-free system.

In one embodiment, the method of screening is performed in a cell-based system.

In one embodiment, the method of screening is performed in vitro.

In one embodiment, the method of screening provides for a first ligand that is oxLDL and the second ligand that is CRP.

In one embodiment, the method of screening provides for a first ligand that is CRP and the second ligand that is oxLDL.

In one embodiment, the method of screening provides for a candidate inhibitor that is selected from the group consisting of a polypeptide, an antibody or a small molecule.

In one embodiment, the method of screening provides for a ligand that is coupled to a detectable label.

In one embodiment, the method of screening provides for a candidate inhibitor that is coupled to a detectable label.

In one embodiment, the method of screening provides that the candidate inhibitor displaces one ligand, but not one other ligand from LOX-1, or wherein the candidate inhibitor interferes with the binding of one ligand, but not one other ligand to LOX-1.

In one embodiment, the method of screening provides that the LOX-1, or the first or second ligand for LOX-1 is attached to a solid support.

In one embodiment, the method of screening provides that the selectivity of the candidate inhibitor for one ligand, but not one other ligand for LOX-1 is determined by an assay selected from the group consisting of an Enzyme Linked Immunoassay (ELISA), fluorescence activated cell sorting, fluorescent resonance energy transfer (FRET), Alphascreen, fluorescence polarization (FP) or surface plasmon resonance (SPR).

One embodiment of the invention provides a method of modulating LOX-1-mediated pro-inflammatory gene expression in a cell comprising contacting the cell with an agent that reduces the levels of oxLDL or CRP, or by contacting the cell with a LOX-I inhibitor.

In one embodiment, the method provides for inhibition of expression of a pro-inflammatory gene that encodes a cytokine, a chemokine, or a cell adhesion molecule in a cell comprising contacting the cell with an agent that reduces the levels of oxLDL or CRP, or by contacting the cell with a LOX-I inhibitor. In one embodiment, the method of the invention provides for inhibiting expression of a cytokine in a cell, wherein the cytokine is interleukin-8, comprising contacting the cell with an agent that reduces the levels of oxLDL or CRP, or by contacting the cell with a LOX-I inhibitor. In one embodiment, the method of the invention provides for inhibiting expression of a cell-adhesion molecule in a cell, wherein the cell-adhesion molecule is ICAM-1 or VCAM-1, comprising contacting the cell with an agent that reduces the levels of oxLDL or CRP, or by contacting the cell with a LOX-I inhibitor. In one embodiment, the method of the invention provides for inhibiting expression of a chemokine in a cell, wherein the chemokine is MCP-1, comprising contacting the cell with an agent that reduces the levels of oxLDL or CRP, or by contacting the cell with a LOX-I inhibitor.

In one embodiment, the method of the invention provides for a LOX-1 inhibitor that prevents the binding of one ligand, but not one other ligand to LOX-1.

In one embodiment, the method of the invention provides for a LOX-1 inhibitor that is selected from the group consisting of a polypeptide, an antibody, a nucleic acid and a small molecule.

In one embodiment, the method of the invention provides for a LOX-1 inhibitor that is a siRNA molecule. In one embodiment, the siRNA molecule comprises the nucleic acid sequence of any of SEQ ID NOs: 25, 26, 27 or 28, or a combination thereof.

In one embodiment, the method provides for a LOX-1 inhibitor that is an antibody selected from a monoclonal antibody, a polyclonal antibody, a chimeric antibody, a single chain antibody, or a fragment of any one of the monoclonal, polyclonal, chimeric or single chain antibodies selected from the group consisting of a mouse antibody, a rat antibody, a goat antibody, a sheep antibody, a rabbit antibody, a porcine antibody, a horse antibody, a human antibody and a humanized antibody. In one embodiment, the antibody is selected from the group consisting of a mouse antibody, a rat antibody, a goat antibody, a sheep antibody, a rabbit antibody, a porcine antibody, a horse antibody, a human antibody and a humanized antibody. In one embodiment, the antibody is a rat anti-mouse LOX-1 antibody.

### BRIEF DESCRIPTION OF THE DRAWINGS

FIGS. 1A and 1B. Demonstrates CRP interaction with ECD of LOX-1 by ELISA.

FIGS. 2A, 2B and 2C. Demonstrates CRP interaction with ECD of LOX-1 by Alphascreen analyses.

FIG. 3. Demonstrates CRP interaction with cell surfaced expressed LOX-1.

FIGS. 4A and 4B. Shows two binding curves of oxLDL and CRP to CHO/LOX-1 cells.

FIG. 5. Demonstrates CRP interaction with endogenously expressed LOX-1 in HAECT-1 cells.

FIG. 6. Demonstrates CRP binding to LOX-1 is distinct from oxLDL binding to LOX-1.

FIG. 7. Demonstrates CRP-mediated gene regulation is inhibited by anti-LOX-1 antibody.

FIG. 8. Shows the Rat Anti-Mouse LOX1-11A2 V_{L} and V_{H} Sequences

FIG. 9. Shows the Rat Anti-Mouse LOX1-33F1V_{L} and V_{H} Sequences

FIG. 10. Shows two binding curves that demonstrate the ability of antibody 11A2 to selectively compete oxidized low density lipoprotein (oxLDL), but not C reactive protein (CRP), binding to a recombinant mouse LOX-1 Fc protein, as shown using an ALPHAscreen binding assay.

FIG. 11. Demonstrates that CRP-mediated gene induction is inhibited by LOX-1 siRNA.

FIG. 12. Demonstrates that LOX-1 activation by oxidized LDL or C reactive protein treatment increases IL-8 expression as shown by microarray analysis.

FIG. 13. Demonstrates that LOX-1 activation by oxidized LDL treatment increases IL-8 expression as shown by ELISA.

### DETAILED DESCRIPTION OF THE INVENTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting.

Accordingly, in the present application, there may be employed conventional molecular biology, microbiology, and recombinant DNA techniques within the skill of the art. Such techniques are explained fully in the literature. See, e.g., Byrd, C M and Hruby, D E, Methods in Molecular Biology, Vol. 269: Vaccinia Virus and Poxyirology, Chapter 3, pages 31-40; Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Although any methods and materials similar or equivalent to those described herein can be used in the practice or testing of the invention, the preferred methods and materials are now described. Unless otherwise specified, all publications mentioned herein are incorporated herein by reference in their entirety.

### DEFINITIONS

The terms used herein have the meanings recognized and known to those of skill in the art, however, for convenience and completeness, particular terms and their meanings are set forth below.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure.

The term “advanced glycation end-products” or “AGEs” refers to a heterogeneous group of molecules formed from the nonenzymatic reaction of reducing sugars with free amino groups of proteins, lipids, and nucleic acids. The initial product of this reaction is called a Schiff base, which spontaneously rearranges itself into an Amadori product, as is the case of the well-known hemoglobin A_{1c} (A1C). These initial reactions are reversible depending on the concentration of the reactants. A lowered glucose concentration will unhook the sugars from the amino groups to which they are attached; conversely, high glucose concentrations will have the opposite effect, if persistent. A series of subsequent reactions, including successions of dehydrations, oxidation-reduction reactions, and other arrangements lead to the formation of AGEs. Several compounds, e.g., ^{ε}N-carboxymethyl-lysine, pentosidine, or methylglyoxal derivatives, serve as examples of well-characterized and widely studied AGEs. In addition to hyperglycemia, oxidative stress can lead to formation of AGEs, such as ^{ε}N-carboxymethyl-lysine.

The term “aged cells”, as used herein, refers to cells that have been incubated at 37° C. for at least 4 days.

In the present invention, the term “anionic phospholipids” refers to phosphatidylserine or phosphatidylinositol.

The term “antibody” is used interchangeably with the term “immunoglobulin” herein, and includes intact antibodies, fragments of antibodies, e.g., Fab, F(ab′)₂ fragments, and intact antibodies and fragments that have been mutated or modified either in their constant and/or variable region (e.g., modifications to produce chimeric, partially humanized, or fully humanized antibodies, as well as mutations to produce antibodies with a desired trait, e.g., reduced FcR binding or altered complement fixation). In addition, mutations or modifications can be made to alter the affinity or avidity of the antibody (e.g., to increase species cross-reactivity or otherwise alter the binding properties of the antibody). An antibody may be isolated from rats, rabbits, goats, sheep, swine, dogs, cats, or horses, for example. An antibody may also be isolated from transgenic animals (e.g., mice) that are capable, upon immunization, of producing a full repertoire of human antibodies in the absence of endogenous immunoglobulin production. See, e.g., Jackobovits et al., Proc. Natl. Acad. Sci. USA, 90:2551 (1993); Jakobovits et al., Nature, 362:255-258 (1993); Bruggermann et al., Year in Immune, 7:33 (1983); and Duchosal et al. Nature 355:258 (1992). Human antibodies can also be derived from phage-display libraries (Hoogenboom et al., J. Mol. Biol. 227:381 (1991); Marks et al., J. Mol. Biol., 222:581-597 (1991); Vaughan et al. Nature Biotech 14:309 (1996)).

Such modification may be readily prepared to include various changes, substitutions, insertions, and deletions. For example, antibody sequences may be optimized for codon usage in the cell type used for antibody expression. To increase the serum half life of the antibody, a salvage receptor binding epitope may be incorporated, if not present already, into the antibody heavy chain sequence. See U.S. Pat. No. 5,739,277. Additional modifications to enhance antibody stability include modification of IgG4 to replace the serine at residue 241 with proline. See Angal et al. (1993) Mol. Immunol. 30: 105-108. Other useful changes include substitutions as required to optimize efficiency in conjugating the antibody with a drug. For example, an antibody may be modified at its carboxyl terminus to include amino acids for drug attachment, for example one or more cysteine residues may be added. The constant regions may be modified to introduce sites for binding of carbohydrates or other moieties.

“Single domain antibodies” can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, cow and shark. According to one aspect of the invention, a single domain antibody as used herein is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

In embodiments where the binding partner is a polypeptide, and more particularly an antibody or a fragment thereof, it can include at least one, or two full-length heavy chains, and at least one, or two light chains. Alternatively, the antibodies or fragments thereof can include only an antigen-binding fragment (e.g., an Fab, F(ab′)₂, Fv or a single chain Fv fragment). The antibody or fragment thereof can be a monoclonal or single specificity antibody. The antibody or fragment thereof can also be a human, humanized, chimeric, CDR-grafted, or in vitro generated antibody. In yet other embodiments, the antibody has a heavy chain constant region chosen from, e.g., IgG1, IgG2, IgG3, or IgG4. In another embodiment, the antibody has a light chain chosen from, e.g., kappa or lambda. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). Typically, the antibody or fragment thereof specifically binds to a predetermined antigen, e.g., a lectin-like oxidized low density lipoprotein receptor (LOX-1) associated with a disease or condition, e.g., atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation.

The term “antibody fragment” refers to a part or portion of an antibody or antibody chain comprising fewer amino acid residues than an intact or complete antibody or antibody chain. Antibody fragments can be obtained via chemical or enzymatic treatment of an intact or complete antibody or antibody chain. Antibody fragments can also be obtained by recombinant means. Exemplary antibody fragments include Fab, Fab′, F(ab′)₂, Fabc, Fd, dAb, and scFv and/or Fv fragments. These antibodies or fragments thereof are included in the scope of the invention, e.g. as “binding partners” that interact with, or bind to, LOX-1, provided that the antibody or fragment inhibits the binding of at least one ligand to LOX-1, but not one other ligand for LOX-1. In certain embodiments, these antibodies or fragments thereof inhibit one or more LOX-1-associated activities (e.g., inhibits binding of a LOX-1 ligand to LOX-1, thereby preventing at least one functional aspect associated with such binding, for example, the induction of inflammatory cytokines, or the formation of reactive oxygen species upon binding of a ligand to LOX-1).

Intact antibodies, are typically tetrameric glycosylated proteins composed of two light (L) chains of approximately 25 kDa each and two heavy (H) chains of approximately 50 kDa each, inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

One of skill in the art will recognize that each subunit structure, e.g., a CH, VH, CL, VL, CDR, FR structure, comprises active fragments, e.g., the portion of the VH, VL, or CDR subunit that binds to the antigen, i.e., the binding fragment, or, e.g., the portion of the CH subunit that binds to and/or activates, e.g., an Fc receptor and/or complement.

Antibodies that are used as binding partners for LOX-1, as described herein, are generally made, for example, via traditional hybridoma techniques (Kohler et al., Nature 256:495-499 (1975)), recombinant DNA methods (U.S. Pat. No. 4,816,567), or phage display techniques using antibody libraries (Clackson et al., Nature 352:624-628 (1991); Marks et al., J. Mol. Biol. 222:581-597 (1991)). For various other antibody production techniques, see Antibodies: A Laboratory Manual, eds. Harlow et al., Cold Spring Harbor Laboratory, 1988.

Further, the antibodies may be tagged or conjugated with a detectable or functional label. These labels include radiolabels (e.g., ¹³¹I or ⁹⁹Tc), enzymatic labels (e.g., horseradish peroxidase or alkaline phosphatase), and other chemical moieties (e.g., biotin), which may be cytotoxic, such as chemotherapy agents (e.g., doxorubicin and vinblastine), as well as more potently cytotoxic derivatives of natural products (e.g., calicheamicin, maytansine, and duocarmycin).

This invention also encompasses binding partners for LOX-1, which are “antigen-binding fragments of antibodies”, wherein the antigen is LOX-1, and which include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) a camelid or camelized variable domain, e.g., a VHH domain; (vii) a single chain Fv (scFv); (viii) a bispecific antibody; and (ix) one or more antigen binding fragments of an immunoglobulin fused to an Fc region. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-26; Huston et al. (1988) Proc. Natl. Acad. Sci. U.S.A. 85:5879-83). Such single chain antibodies are also intended to be encompassed within the term “antigen-binding fragment” of an antibody. These antibody fragments are obtained using conventional techniques known to those skilled in the art, and the fragments are evaluated for function in the same manner as are intact antibodies.

The inventive further encompasses binding partner that are shark IgNARs; see, e.g., Dooley et al., Proc. Natl. Acad. Sci. U.S.A., 103:1846-1851 (2006).

An “apoptotic cell” refers to a cell that has undergone “apoptosis”, which refers to programmed cell death and is characterized by membrane blebbing, chromatin condensation and fragmentation, and formation of apoptotic bodies. Degradation of genomic DNA during apoptosis results in formation of characteristic, nucleosome sized DNA fragments; this degradation produces a diagnostic (about) 180 bp laddering pattern when analyzed by gel electrophoresis. A later step in the apoptotic process is degradation of the plasma membrane, rendering apoptotic cells leaky to various dyes (e.g., trypan blue and propidium iodide). Specific markers for apoptosis include, but are not limited to, annexin V staining, DNA laddering, staining with dUTP and terminal transferase [TUNEL].

The term “bacterial cells” refers to any gram positive or gram negative microorganism that acts as a ligand for LOX-1.

The term “bile salt-dependent lipase” is an enzyme normally present in blood, but which originates from exocrine pancreatic secretion. This enzyme may have pathophysiologic relevance in atherosclerosis.

As used herein in connection with the design or development of binding partners of LOX-1, the term “bind” and “binding” and like terms refer to a non-convalent energetically favorable association between the specified molecules (i.e., the bound state has a lower free energy than the separated state, which can be measured calorimetrically). For binding to a target, the binding is at least selective, that is, the binding partner binds preferentially to a particular target or to members of a target family at a binding site, as compared to non-specific binding to unrelated proteins not having a similar binding site. For example, BSA is often used for evaluating or controlling for non-specific binding. In addition, for an association to be regarded as binding, the decrease in free energy going from a separated state to the bound state must be sufficient so that the association is detectable in a biochemical assay suitable for the molecules involved. Binding partners can be characterized by their affinity for the target molecule as measured by determining the dissociation constant or by measuring their effect on the activity of the target molecule.

In addition, the interaction of a ligand with its target structure can be assessed by measurement of the free energy in kcal/mol using approaches that involve AutoDock, Molecular Dynamics (MD) and Molecular Mechanics/Poisson-Boltzmann Solvent Accessible surface area (MM-PBSA) calculations. This may be done using the crystal structures of the ligand binding domains of the receptor, if known. Molecular docking is used to generate several distinct binding orientations. Molecular dynamics simulation is used to further relax the complex. MM-PBSA is then used to estimate the affinity for each binding mode. The binding modes with the lowest free energy are expected to be the most favorable. The energetically most favorable binding mode would provide for a free energy of <0 kcal/mol (negative value), and larger the free energy, the less favorable the interaction. The binding between a ligand and its target is unique in each system and cannot be compared to other systems. However, it can be stated that the smaller the free energy the more favorable the binding. For favorable interactions, the free energy is negative. The more negative, the more favorable the interaction is.

The term “binding partner”, as used in the context of the present invention, relates primarily to polypeptides, antibodies or fragments thereof, and small synthetic, semi-synthetic or naturally derived molecules/compounds that bind to LOX-1, but may also refer to other molecules that bind to LOX-1. In one embodiment, the “binding partner” inhibits the binding of one ligand to LOX-1, but does not inhibit the binding of one other ligand to LOX-1. In one embodiment, a “binding partner” may comprise a “binding portion” of an antibody (or “antibody portion”). The term “binding portion” or “antibody portion” includes one or more complete domains, e.g., a pair of complete domains, as well as fragments of an antibody that retain the ability to specifically bind to LOX-1. It has been shown that fragments of a full-length antibody can perform the binding function of an antibody.

Binding fragments may be produced by recombinant DNA techniques, or by enzymatic or chemical cleavage of intact immunoglobulins. Moreover, antibodies, antibody portions and immunoadhesion molecules can be obtained using standard recombinant DNA techniques, as described herein and as known in the art. Binding fragments include Fab, Fab′, F(ab′)₂, Fabc, Fd, dAb, Fv, single chains, single-chain antibodies, e.g., scFv, and single domain antibodies (Muyldermans et al., 2001, 26:230-5), and an isolated complementarity determining region (CDR). Single chain antibodies may be considered as a “binding partner”, as described herein, as they are considered to fall within the term “binding portion” of an antibody. Other forms of single chain antibodies, such as diabodies may also be considered as binding partners for use in the methods of the invention. An antibody or binding portion thereof also may be part of a larger immunoadhesion molecules formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

Other than “bispecific” or “bifunctional” antibodies, an antibody is understood to have each of its binding sites identical. A “bispecific” or “bifunctional antibody” is an artificial hybrid antibody having two different heavy/light chain pairs and two different binding sites. A bispecific antibody can also include two antigen binding regions with an intervening constant region. Bispecific antibodies can be produced by a variety of methods including fusion of hybridomas or linking of Fab′ fragments. See, e.g., Songsivilai et al., Clin. Exp. Immunol. 79:315-321, 1990; Kostelny et al., 1992, J. Immunol. 148, 1547-1553.

The terms “cell”, or “cells”, and the like, as used herein, is intended to include any individual cell or cell culture (a “population of cells”), which expresses LOX-1. The terms “cell”, or “cells”, may include the progeny of a single cell; however, the progeny may not necessarily be completely identical (in morphology or in genomic or total DNA complement) to the original parent cell due to natural, accidental, or deliberate mutation. The cells may be eukaryotic cells, and may include, but are not limited to, mammalian cells, such as endothelial cells, macrophages, monocytes, dendritic cells, vascular smooth muscle cells (SMC), chondrocytes, platelets, intestinal cells and cardiac myocytes.

The term “cellular ligand” refers to any cell that acts as a ligand for LOX-1.

It is noted that in this disclosure, terms such as “comprises”, “comprised”, “comprising”, “contains”, “containing” and the like can have the meaning attributed to them in U.S. patent law; eg., they can mean “includes”, “included”, “including” and the like. Terms such as “consisting essentially of” and “consists essentially of” have the meaning attributed to them in U.S. patent law, eg., they allow for the inclusion of additional ingredients or steps that do not detract from the novel or basic characteristics of the invention, ie., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, eg., novel, nonobvious, inventive, over the prior art, eg., over documents cited herein or incorporated by reference herein. And, the terms “consists of” and “consisting of” have the meaning ascribed to them in U.S. patent law; namely, that these terms are closed ended.

The term “diabetes mellitus” refers to high blood sugar or ketoacidosis, as well as chronic, general metabolic abnormalities arising from a prolonged high blood sugar status or a decrease in glucose tolerance. “Diabetes mellitus” encompasses both the type I and type II (Non Insulin Dependent Diabetes Mellitus or NIDDM) forms of the disease. The risk factors for diabetes include the following factors: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

A “disease or condition associated with elevated levels of LOX-1 or a LOX-1 ligand” includes any disease or condition treatable with a binding partner for LOX-1, wherein the binding partner inhibits the binding of at least one ligand to LOX-1, but not one other ligand for LOX-1. The binding/interaction of the binding partner, for example, an antibody as described herein, to LOX-1 results in the inhibition of certain detrimental effects associated with the binding of at least one ligand to LOX-1, but allows or retains other non-detrimental effects associated with the binding of one other ligand to LOX-1. In certain embodiments, such selectivity may be advantageous for treating a “disease or condition associated with elevated levels of LOX-1 or a LOX-1 ligand” including diseases or conditions such as atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation.

The term “domain” refers to a globular region of a heavy or light chain polypeptide comprising peptide loops (e.g., comprising 3 to 4 peptide loops) stabilized, for example, by beta.-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain variable regions”, “heavy chain variable domains”, “VH” regions or “VH” domains).

The phrase “effective amount” or “therapeutically effective amount”, as used herein, means that amount of one or more agent, material, or composition comprising one or more agents of the present invention that is effective for producing some desired effect in an animal. It is recognized that when an agent is being used to achieve a therapeutic effect, the actual dose which comprises the “therapeutically effective amount” will vary depending on a number of conditions including the particular condition being treated, the severity of the disease, the size and health of the patient, the route of administration, etc. A skilled medical practitioner can readily determine the appropriate dose using methods well known in the medical arts.

“Elevated levels of LOX-1 or a LOX-1 ligand” are established by determining the amount of LOX-1 or at least one LOX-1 ligand, as described herein, in a tissue or body fluid, such as, blood (whole blood, blood cells or plasma or serum) of normal patients who do not suffer from a disease, condition or disorder, associated with elevated levels of a LOX-1 ligand and comparing these levels with that of patients who suffer from any disease or disorder associated with elevated levels of a LOX-1 ligand. As noted in the present invention, the ligands may be any modified low density lipoprotein, such as oxidized LDL. The ligand may be C reactive protein or an advanced glycation endproduct, or an apoptotic cell. The skilled artisan would be cognizant of the procedures available for measurement of these ligands in a tissue sample or a body fluid. Furthermore, the diseases or disorders associated with elevated levels of a LOX-1 ligand include, but are not limited to atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation.

By “endothelial cell dysfunction” is meant the inability of an endothelial cell to maintain its normal function. Non-limiting examples of endothelial cell function include maintaining balanced vascular tone, inhibiting thrombosis, inhibiting pro-inflammatory processes, maintaining vascular integrity (e.g., non-leakiness of the vasculature), and maintaining an anti-proliferative state in both the endothelium and the surrounding smooth muscle cells. The endothelial cell functions ensure proper vascular pressure, patency, and perfusion. Endothelial cell dysfunction is generally characterized by, for example, the increased adherence of mononuclear cells to the endothelium, the stimulation of macrophage lipoprotein lipase (LPL) production and overexpression of LPL, accelerated apoptosis, phosphorylation of vasodilator-stimulated phosphoprotein (VASP) and leukocyte adhesion through intercellular adhesion molecules (for example, ICAM-1). An endothelial cell disorder is any disorder that is characterized by endothelial cell dysfunction. Non-limiting examples of diseases or disorders that are characterized by endothelial cell dysfunction include angiogenic disorders such as cancers which require neovascularization to support tumor growth, infectious diseases, autoimmune disorders, vascular malformations, DiGeorge syndrome, HHT, cavernous hemangioma, transplant arteriopathy, vascular access stenosis associated with hemodialysis, vasculitis, vasculitidis, vascular inflammatory disorders, atherosclerosis, obesity, psoriasis, warts, allergic dermatitis, scar keloids, pyogenic granulomas, blistering disease, Kaposi sarcoma, persistent hyperplastic vitreous syndrome, retinopathy of prematurity, choroidal neovascularization, macular degeneration, diabetic retinopathy, ocular neovascularization, primary pulmonary hypertension, asthma, nasal polyps, inflammatory bowel and periodontal disease, ascites, peritoneal adhesions, contraception, endometriosis, uterine bleeding, ovarian cysts, ovarian hyperstimulation, arthritis, rheumatoid arthritis, chronic articular rheumatism, synovitis, osteoarthritis, osteomyelitis, osteophyte formation, sepsis, and vascular leak. Endothelial cell dysfunction can be determined using assays known in the art including detecting the increased expression of endothelial adhesion molecules or decreased expression or biological activity of nitric oxide synthase (eNOS).

“Fragment” refers to either a protein or polypeptide comprising an amino acid sequence of at least 4 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid.

The term “isolated” or “purified” means that the material is removed from its original environment (e.g., the natural environment if it is naturally occurring). For example, an “isolated” or “purified” polypeptide or protein or antibody, e.g. an “isolated binding partner” or “isolated antibody” is substantially free of cellular material or other contaminating proteins from the cell or tissue source from which the protein or antibody is derived, or substantially free of chemical precursors or other chemicals when chemically synthesized. The language “substantially free of cellular material” includes preparations of a polypeptide/protein or antibody in which the polypeptide/protein or antibody is separated from cellular components of the cells from which it is isolated or recombinantly produced. Thus, a polypeptide/protein or antibody that is substantially free of cellular material includes preparations of the polypeptide/protein or antibody having less than about 30%, 20%, 10%, 5%, 2.5%, or 1%, (by dry weight) of contaminating protein. When the polypeptide/protein or antibody is recombinantly produced, it is also preferably substantially free of culture medium, i.e., culture medium represents less than about 20%, 10%, or 5% of the volume of the protein or antibody preparation. When polypeptide/protein or antibody is produced by chemical synthesis, it is preferably substantially free of chemical precursors or other chemicals, i.e., it is separated from chemical precursors or other chemicals which are involved in the synthesis of the protein. Accordingly, such preparations of the polypeptide/protein or antibody have less than about 30%, 20%, 10%, 5% (by dry weight) of chemical precursors or compounds other than polypeptide/protein or antibody or fragment of interest. Proteins or polypeptides referred to herein as “recombinant” are proteins or polypeptides produced by the expression of recombinant nucleic acids.

An “isolated antibody” includes an antibody that is substantially free of other antibodies having different antigenic specificities. An isolated antibody that specifically binds LOX-1 may bind LOX-1 molecules from other species. Moreover, an isolated antibody may be substantially free of other cellular material and/or chemicals.

The term “lectin-like oxidized low density lipoprotein” or “LOX-1” refers to a 50 kDa type II membrane protein that structurally belongs to the C-type lectin family with a short intracellular N-terminal hydrophilic and a long extracellular C-terminal hydrophilic domain separated by a hydrophobic domain of 26 amino acids. LOX-1 is the primary oxLDL receptor in endothelial cells and as such, mediates most of the toxic effects of ox-LDL. LOX-1 is also present on other cell types, including, but not limited to, macrophages, monocytes, dendritic cells, vascular smooth muscle cells (SMC), chondrocytes, intestinal cells and cardiac myocytes. LOX-1 was identified in bovine aortic endothelial cells and sequenced by Sawamura et al. (Sawamura, T, et al. Nature, 1997, 386:73-77). Mouse LOX-1 was cloned by Hoshikawa, H. et al. (Hoshikawa, H. et al., Biochem Biophys Res Corn, 1998, Vol. 245(3):841-846). The human LOX-1 sequence can be found in GenBank accession number NM_{—}002543.

The term “ligand”, as used in the present invention, refers to a substance, for example, a small molecule that is capable of specifically binding to a larger molecule, for example, a receptor. In the present invention, the receptor is LOX-1 and the ligand may be selected from a modified lipoprotein, such as oxidized low density lipoprotein (oxLDL), acetylated LDL (AcLDL), advanced glycosylation end-products or advanced glycation end-products (used interchangeably and referred to as “AGEs”). In one embodiment, the ligand may be an anionic phospholipid, such as phosphatidylserine or phosphatidylinositol. In one embodiment, the ligand may be an apoptotic cell, an aged cell, an activated platelet or a bacterial cell.

The term “LOX-1 mediated” refers to the fact that the LOX-1 receptor mediates the cellular response upon binding of a LOX-1 ligand, such as CRP or oxLDL to LOX-1 on the cell surface, which then triggers the cell to increase production of certain pro-inflammatory molecules.

In the present invention, the term “modified lipoprotein” refers to any modification of a lipoprotein, for example, a low density lipoprotein, by an oxidative process on either the lipid or protein constituents of the LDL.

The term “modulating” is used to reflect either a positive or a negative change in a cell after binding of a LOX-1 ligand to the LOX-1 receptor. In one embodiment of the present invention, and as used herein in the phrase “modulating a LOX-1-mediated pro-inflammatory gene expression in a cell”, the term “modulating” refers to the inhibition of the expression or synthesis of a pro-inflammatory gene encoding, for example, a cytokine, a chemokine, or a cell adhesion molecule, with particular emphasis on inhibiting those molecules that may play a role in an inflammatory response. The inhibition of expression or synthesis of the pro-inflammatory gene may occur by contacting the cell with an agent that inhibits or blocks binding of a LOX-1 ligand to the cell, for example, an antibody, a polypeptide, a nucleic acid (for example, an siRNA), or a small molecule. Inhibitors are also compounds that decrease, block, prevent, delay activation, inactivate, desensitize, or down regulate LOX-1 expression or function. An inhibitor may also be an agent that reduces the levels of a LOX-1 ligand, such as CRP or oxLDL, or an agent that prevents the synthesis or expression of a LOX-1 ligand, such as CRP or oxLDL.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline, (18) Ringer's solution, (19) ethyl alcohol; (20) phosphate buffer solutions; and (21) other non-toxic compatible substances employed in pharmaceutical formulations.

The term “pro-inflammatory gene” refers to a gene encoding any molecule, such as, but not limited to, a cytokine, a chemokine, or a cell-adhesion molecule, which plays a role in an inflammatory process. Exemplary “pro-inflammatory” genes include, but are not limited to, interleukin-8 (IL-8), intercellular adhesion molecule-1 (ICAM-1), vascular cell adhesion molecule-1 (VCAM-1) and monocyte chemotactic protein-1 (MCP-1).

The terms “protein”, “polypeptide” and “peptide” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include modifications, such as deletions, additions and substitutions (generally conservative in nature, but which may be non-conservative), to a native sequence, preferably such that the protein maintains the ability to elicit an immunological response within an animal to which the protein is administered. Also included are post-expression modifications, eg. glycosylation, acetylation, phosphorylation and the like.

The term “selective” or “selectively” as used herein means having the characteristic or property of being highly specific in binding, activity, or effect. In the present invention, binding partners, such as an antibodies, are described as selective for inhibiting the binding of one ligand to LOX-1 over other ligands, for example, preferential inhibition of binding of oxLDL to LOX-1, but not CRP binding to LOX-1. The degree of selectivity may vary, but in many embodiments, a selective binding partner would be at least tenfold selective for the desired target. In certain embodiments, the binding partner, for example, an antibody, would be 100- to 1000-fold selective.

A “small molecule” refers to a composition that has a molecular weight of less than 3 kilodaltons (kDa), and preferably less than 1.5 kilodaltons, and more preferably less than about 1 kilodalton. Small molecules may be nucleic acids, peptides, polypeptides, peptidomimetics, carbohydrates, lipids or other organic (carbon-containing) or inorganic molecules. Small molecules may be synthetic, semi-synthetic, or naturally derived. As those skilled in the art will appreciate, based on the present description, extensive libraries of chemical and/or biological mixtures, often fungal, bacterial, or algal extracts, may be screened with any of the assays of the invention to identify compounds that act as a binding partner that inhibits the binding of at least one ligand to LOX-1, but not one other ligand for LOX-1. A “small organic molecule” is an organic compound (or organic compound complexed with an inorganic compound (e.g., metal)) that has a molecular weight of less than 3 kilodaltons, and preferably less than 1.5 kilodaltons, and more preferably less than about 1 kilodalton.

“Specific binding” of for example, a “binding partner”, such as an antibody to LOX-1, means that the antibody exhibits appreciable affinity for a particular antigen or epitope and, generally, does not exhibit significant crossreactivity. The term “anti-LOX-1 antibody” as used herein refers to an antibody that binds specifically to LOX-1. The antibody may exhibit no crossreactivity (e.g., does not crossreact with non-LOX-1 peptides. “Appreciable” binding includes binding with an affinity of at least 10⁶, 10⁷, 10⁸, 10⁹ M⁻¹, or 10¹⁰ M⁻¹. Antibodies with affinities greater than 10⁷ M⁻¹ or 10⁸ M⁻¹ typically bind with correspondingly greater specificity. Values intermediate of those set forth herein are also intended to be within the scope of the present invention and antibodies of the invention bind to LOX-1 with a range of affinities, for example, 10⁶ to 10¹⁰ M⁻¹, or 10⁷ to 10¹⁰ M⁻¹, or 10⁸ to 10¹⁰ M⁻¹. An antibody that “does not exhibit significant crossreactivity” is one that will not appreciably bind to an entity other than its target (e.g., a different epitope or a different molecule). For example, an antibody that specifically binds to LOX-1 will appreciably bind LOX-1 but will not significantly react with non-LOX-1 proteins or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

A “therapeutic antibody” relates to any of the above antibody molecules, either alone or coupled to a moiety that allows for targeting to a particular receptor, such as LOX-1, or cell type, or to the site of injury, or coupled to a chemical or protein moiety that allows for enhanced uptake by a cell, whereby such therapeutic antibody is used to treat a disease or to ameliorate at least one symptom associated with the disease.

As used herein, “treatment” (including variations thereof, for example, “treat”, “treating” or “treated”) refers to any one or more of the following: (i) the prevention of a disease or condition, (ii) the reduction in the severity of, or, in the elimination of at least one symptom of the disease, and (iii) the substantial or complete elimination of the disorder in question. Hence, treatment may be effected prophylactically (prior to getting the disease) or therapeutically (following exhibiting at least one symptom of the disease). According to a particular embodiment of the present invention, compositions and methods are provided which treat, including prophylactically and/or therapeutically, a host animal against a disease resulting from increased levels of certain ligands that bind to LOX-1.

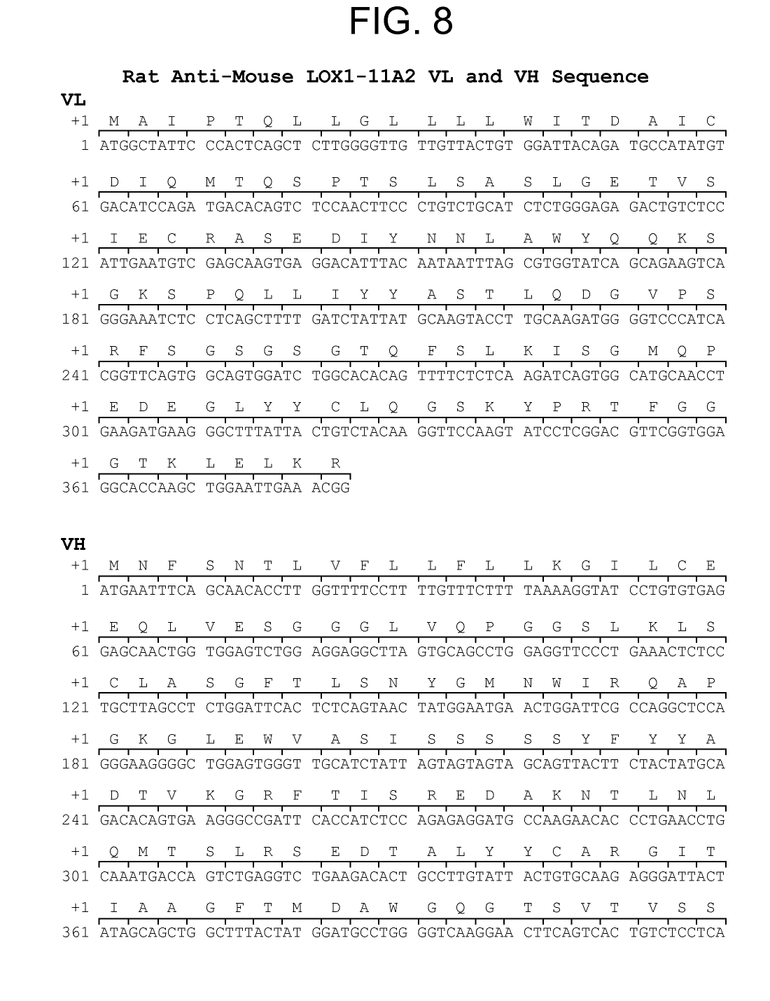

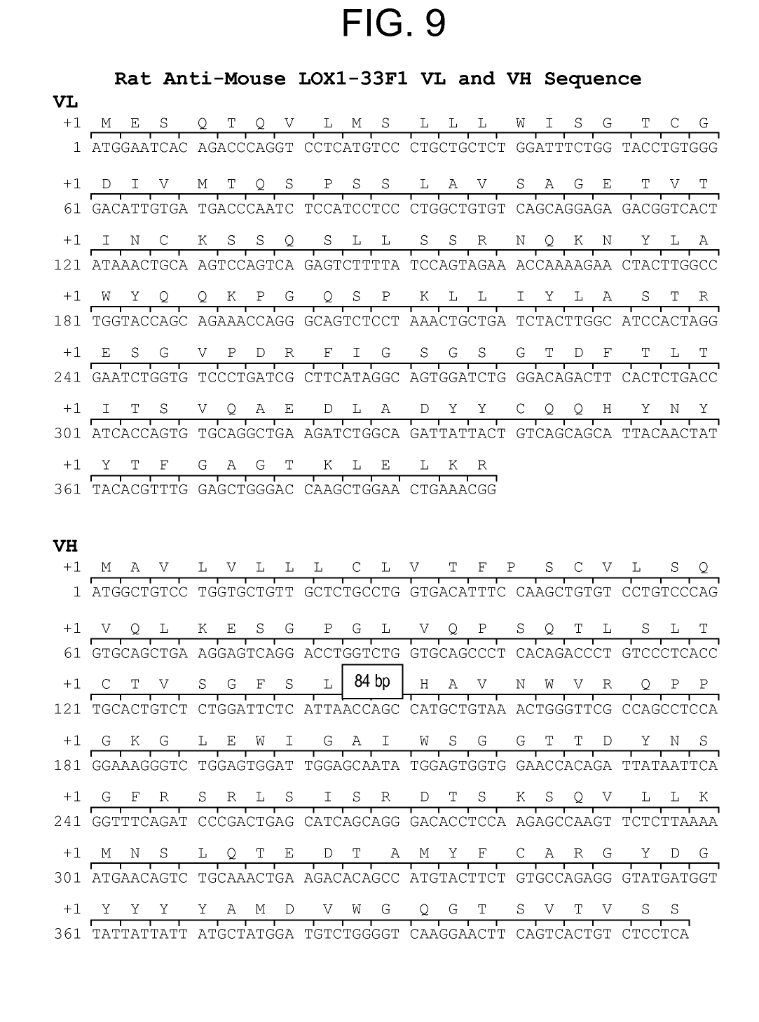

The “V_{L}” or “variable light chain” of the binding partners described herein, e.g. the LOX-1 specific antibodies, refers to the variable light chain domain of two rat anti-mouse LOX-1 antibodies, designated LOX1-11A2 and LOX1-33F1, as shown in FIGS. 8 and 9 and in SEQ ID NOs: 1, 2, 5 and 6. The “V_{H}” or “variable heavy chain” of the binding partners described herein, e.g. the LOX-1 specific antibodies, refers to the variable heavy chain domain of two LOX-1 antibodies, designated LOX1-11A2 and LOX1-33F1, as shown in FIGS. 8, 9 and 10 and in SEQ ID NOs: 3, 4, 7 and 8.

A number of circulating soluble factors are involved in the induction and amplification of vascular inflammation, a condition characterized by endothelial dysfunction and leukocyte adhesion and infiltration. Among these soluble factors, C-reactive protein (CRP) was initially identified as an acute phase serum marker of inflammation but is now recognized as a strong risk factor for cardiovascular events (Verma S, Szmitko P E, Ridker P M. Nature Clinical Practice Cardiovascular Medicine. 2005; 2:29-36). Whether CRP plays a causal role in cardiovascular disease is still debated but it has been shown to have direct pro-inflammatory effects on vascular endothelial cells and leukocytes with modulation of cytokine expression, nitric oxide signaling, inflammatory gene expression and leukocyte adhesion (Devaraj S, Xu D Y, Jialal I. Circulation. 2003; 107:398-404; Venugopal S K, Devaraj S, Jialal I. Current Opinion in Nephrology & Hypertension. 2005; 14:33-37; Wang Q, Zhu X, Xu Q, Ding X, Chen Y E, Song Q. American Journal of Physiology—Heart & Circulatory Physiology. 2005; 288:H1539-1545; Pasceri V, Willerson J T, Yeh E T. Circulation. 2000; 102:2165-2168; Devaraj S, Davis B, Simon S I, Jialal I. American Journal of Physiology—Heart & Circulatory Physiology. 2006; 291:H1170-1176).

The Fcγ receptors FcγRI, FcγRIIa and FcγRIIb have been identified as CRP receptors on leukocytes, vascular smooth muscle cells and endothelial cells (Venugopal S K, Devaraj S, Jialal I. Current Opinion in Nephrology & Hypertension. 2005; 14:33-37) and this interaction has been shown to mediate several CRP-mediated inflammatory functions including eNOS dysregulation, superoxide generation and increased ICAM-1 and VCAM-1 expression (Devaraj S, Davis B, Simon S I, Jialal I. American Journal of Physiology—Heart & Circulatory Physiology. 2006; 291:H1170-1176; Devaraj S, Du Clos T W, Jialal I. Arterioscler Thromb Vasc Biol. 2005; 25:1359-1363; Mineo C, Gormley A K, Yuhanna I S, Osborne-Lawrence S, Gibson L L, Hahner L, Shohet R V, Black S, Salmon J E, Samols D, Karp D R, Thomas G D, Shaul P W. Circulation Research. 2005; 97:1124-1131; Ryu J, Lee C W, Shin J A, Park C S, Kim J J, Park S J, Han K H. Cardiovascular Research. 2007; 75:555-565) lending credence for the causal role of CRP in cardiovascular disease.

LOX-1 is known to promote vascular inflammation and endothelial dysfunction and consequently is thought to play a pathogenic role in diseases such as heart failure, myocardial injury, diabetic nephropathy, hypertension, sepsis, osteoarthritis and rheumatoid arthritis. LOX-1 may also impact other disease processes, since LOX-1 binds other ligands including platelets, aged RBCs, apoptotic cells and advanced glycation end products. The expression of LOX-1 was initially described in endothelial cells (ECs), but has been demonstrated on numerous other cell types such as macrophages, smooth muscle cells and platelets. Based upon a potential beneficial action of LOX-1 in promoting its scavenging functions, such as the elimination of apoptotic cells from the circulation, it may be harmful to eliminate all LOX-1 functions. Therefore, identification of ligand selective LOX-1 antibodies may provide selectivity of action against only that pathogen/ligand that is promoting disease progression.

LOX-1 activation by oxLDL has been shown to stimulate NF-κB and MAPK pathways, generate reactive oxygen species and inhibit nitric oxide production which all leads to endothelial dysfunction. LOX-1 inhibition via blocking antibodies or antisense technology is associated with attenuation of sepsis, heart failure, rheumatoid arthritis, atherosclerosis and the associated ischemic injury. Therefore, LOX-1, and oxLDL selective LOX-1 antibodies, may be a novel target for drug therapy.

Accordingly, the present invention relates to methods and compositions for selectively inhibiting the binding of certain ligands to LOX-1, while retaining the binding of other ligands to LOX-1. In particular, the present invention relates to the identification of ligand selective lectin-like receptor for oxidized low-density lipoprotein (LOX-1) antibodies for use in the treatment of diseases associated with elevated levels of various LOX-1 ligands.

C-reactive protein (CRP) is a risk factor for cardiovascular events and functions to amplify vascular inflammation through promoting endothelial dysfunction (See Verma S, Szmitko P E, Ridker P M. Nature Clinical Practice Cardiovascular Medicine. 2005; 2:29-36). Lectin-like oxidized low-density lipoprotein (oxLDL) receptor-1 (LOX-1) is the primary endothelial receptor for oxLDL and both its expression and function are associated with vascular inflammation. As a scavenger receptor, LOX-1 is capable of binding to a variety of structurally unrelated ligands. The present invention demonstrates that CRP can act as a novel ligand for LOX-1. The direct interaction between these two proteins was demonstrated with purified protein in both ELISA and alphascreen assays. This interaction could be disrupted with known LOX-1 ligands, such as oxLDL and carrageenan. Moreover, the CRP interaction with cell surface expressed LOX-1 was confirmed in cell-based immunofluorescent binding studies. Mutagenesis studies demonstrated that the arginine residues forming the basic spine structure on the LOX-1 ligand binding interface were dispensable for CRP binding, suggesting a novel ligand binding mechanism for LOX-1 distinct from that used for oxLDL binding. Treatment of human endothelial cells with CRP led to activation of pro-inflammatory genes including LOX-1, MCP-1, and VCAM-1 and were LOX-1 dependent as demonstrated by the antagonism observed with an anti-LOX-1 antibody. The present examples identify and characterize the direct interaction between LOX-1 and CRP and suggest that this interaction may mediate CRP-induced endothelial dysfunction.

More particularly, the examples presented herein demonstrate that CRP can directly interact with LOX-1 and the binding mechanism for LOX-1 appears to be distinct from oxLDL interaction. In human aortic endothelial cells (HAECT-1), CRP treatment results in the elevation in LOX-1 and its downstream gene targets that is sensitive to inhibition by an anti-LOX-1 antibody. The results shown here suggest that LOX-1 is a novel receptor for CRP and that some of the pathologic activities associated with CRP may be mediated by LOX-1 downstream signaling. The examples presented herein further demonstrate that antibodies to LOX-1 may be generated, which selectively inhibit interaction of oxLDL with LOX-1, but which do not inhibit the interaction of C reactive protein to LOX-1. Therefore, the present invention demonstrates that it is possible to generate antibodies against LOX-1 that may have selective biologic action.

LOX-1 was originally identified as a major receptor for oxidized low density lipoprotein (oxLDL) in endothelial cells (Sawamura T. et al. Nature 1997; 386:73-77). It has since been identified on many other cell types, including macrophages, monocytes, vascular smooth muscle cells (SMC), cultured rat and human chondrocytes and human INT407 intestinal cells (Sawamura T. et al, supra; Mingyi C. et al. Pharmacol. Ther. 2002, 95:89-100; Kakutani, M. et al. Biochem Biophys Res Comm 2001, 282:180-185). The methods of the present invention are carried out using cells as described above, which express LOX-1 endogenously, or cells into which the nucleic acid encoding LOX-1 is inserted using standard methods known to those skilled in the art. For example, representative host cells include mammalian and human cells, such as Chinese Hamster Ovary (CHO) cells, HEK-293 cells, HeLa cells, CV-1 cells, and COS cells. Methods for generating a stable cell line following transformation of a heterologous construct into a host cell are known in the art. Representative non-mammalian host cells include insect cells (Potter et al. (1993) Int. Rev. Immunol. 10(2-3):103-112).

Several ligands for LOX-1 have been identified. These include, but are not limited to, a modified lipoprotein, an anionic phospholipid, a cellular ligand, a bile salt-dependent lipase and C-reactive protein. More particularly, the modified lipoprotein may be selected from oxidized low density lipoprotein (ox-LDL), acetylated low density lipoprotein (Ac-LDL), and advanced glycation end-products (AGEs). The anionic phospholipids may be selected from phosphatidylserine or phosphatidylinositol. The cellular ligand may be selected from apoptotic cells, aged cells, activated platelets and bacterial cells.

The present invention provides methods for selectively inhibiting binding of one ligand for LOX-1 but not inhibiting the binding of one other ligand for LOX-1. Accordingly, the present invention utilizes various assays for identifying and/or generating binding partners for LOX-1 that exhibit this selective inhibition. The binding partners so identified may be polypeptides, antibodies, or small molecules, as described herein. The small molecules may be synthetic, semi-synthetic or naturally derived compounds. The binding partners identified may be used for treating diseases or conditions associated with elevated levels of LOX-1 and/or LOX-1 ligands, or may be used for diagnosing certain diseases or conditions associated with elevated levels of LOX-1 or LOX-1 ligands. These elevated levels may be the result of one or many factors, including, but not limited to, chemicals, inflammatory cytokines, stress, or a pathological condition. Elevated LOX-1 and/or LOX-1 ligands are found in many disease states, including, but not limited to, atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation.

In one embodiment, binding partners that selectively interact with (e.g., bind to) and block or antagonize the binding of one ligand, but not one other ligand to LOX-1, are identified in a cell-based assay system. In accordance with this embodiment, cells expressing LOX-1, a fragment of LOX-1, LOX-1 related polypeptide, or a binding fragment thereof, are contacted with a candidate binding partner or a control compound and the ability of the candidate binding partner to interact with LOX-1 or fragment thereof is determined. Alternatively, the ability of a candidate binding partner to compete for binding with a known ligand or compound known to bind LOX-1 is measured. If desired, this assay may be used to screen a plurality (e.g. a library) of candidate binding partners. The cell, for example, can be of eukaryotic origin (e.g., yeast, insect or mammalian). Further, the cells can express LOX-1 endogenously or be genetically engineered to express LOX-1, a binding fragment or a LOX-1 fusion protein. In some embodiments, LOX-1 or a fragment thereof, or the candidate binding partner is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between LOX-1 and a candidate binding partner. The ability of the candidate binding partner to interact directly or indirectly with LOX-1 or fragment thereof or a fusion protein or to modulate the activity of LOX-1 can be determined by methods known to those of skill in the art. For example, the interaction or modulation by a candidate binding partner can be determined by flow cytometry, a scintillation assay, immunoprecipitation or western blot analysis, based on the present description, or by a competitive radioreceptor assay.

Another method includes the exposure of cells expressing LOX-1 to a candidate LOX-1 binding partner, and determining the duration and intensity of the response (for instance, the function of the cells in culture) in the presence of the candidate binding partner and comparing the duration and intensity to that response in the absence of the candidate binding partner or in the presence of a known LOX-1 ligand. The comparison step of the invention can be preferably performed directly, i.e., by comparing the culture's response to the candidate LOX-1 binding partner to that of a known LOX-1 binding partner in a contemporaneous parallel culture. Alternatively, the comparison can be made with a historical control showing an effect on cell function or release of inflammatory mediators that is comparable to that observed under the same conditions with the culture and a known LOX-1 binding partner.

In one embodiment, the comparison is performed longitudinally. Replicate cultures, i.e., at least duplicate, are established and the candidate binding partner is introduced into the cultures. The response of the cultures at time points that are shortly after the introduction and before and at or after some time (for instance one hour) following the introduction is determined. A LOX-1 binding partner can be identified by the persistence of the response by comparison to a contemporaneous control.

Selecting the candidate binding partner that interacts with or binds to LOX-1 or otherwise inhibits or blocks binding of a known ligand for LOX-1 may be performed in multiple ways. The candidate binding partners may first be chosen based on their structural and functional characteristics, using one of a number of approaches known in the art. For instance, homology modeling can be used to screen small molecule libraries in order to determine which molecules would be candidates to interact with LOX-1 thereby selecting plausible targets. See neogenesis.com for a commercially available screening of compounds using multiple different approaches such as an automated ligand identification system and quantized surface complementarity. The candidate binding partners to be screened can include both natural and synthetic compounds. Furthermore, any desired compound may be examined for its ability to interact with or bind to LOX-1 including as described below.

Binding to or interaction with LOX-1 may be determined by performing an assay such as, e.g., a binding assay between a desired binding partner and LOX-1. In one aspect, this is done by contacting the binding partner to LOX-1 and determining its dissociation rate. Numerous possibilities for performing binding assays are well known in the art. The indication of a compound's ability to bind to LOX-1 is determined, e.g., by a dissociation rate, and the correlation of binding activity and dissociation rates is well established in the art. For example, the assay may be performed by radio-labeling a reference compound, or other suitable radioactive marker, and incubating it with the cell bearing LOX-1. Test compounds are then added to these reactions in increasing concentrations. After optimal incubation, the reference compound and receptor complexes are separated, e.g., with chromatography columns, and evaluated for bound ¹²⁵I-labeled peptide with a gamma (γ) counter. The amount of the test compound necessary to inhibit 50% of the reference compound's binding is determined. These values are then normalized to the concentration of unlabeled reference compound's binding (relative inhibitory concentration (RIC)⁻¹=concentrationₜₑₛₜ/concentration_{refeence}). A small RIC⁻¹ value indicates strong relative binding, whereas a large RIC⁻¹ value indicates weak relative binding. See, for example, Latek et al., Proc. Natl. Acad. Sci. USA, Vol. 97, No. 21, pp. 11460-11465, 2000. A LOX-1 binding partner may be computationally evaluated and designed by means of a series of steps in which chemical groups or fragments are screened and selected for their ability to associate with the individual binding pockets or interface surfaces of the protein (e.g. LOX-1). One skilled in the art may employ one of several methods to screen chemical groups or fragments for their ability to associate with LOX-1. This process may begin by visual inspection of, for example, the protein/protein interfaces or the binding site on a computer screen based on the available crystal complex coordinates of LOX-1, including a protein known to interact with LOX-1. Selected fragments or chemical groups may then be positioned in a variety of orientations, or docked, at an individual surface of LOX-1 that participates in a protein/protein interface or in the binding pocket. Docking may be accomplished using software such as QUANTA and SYBYL, followed by energy minimization and molecular dynamics with standard molecular mechanics forcefields, such as CHARMM and AMBER (AMBER, version 4.0 (Kollman, University of California at San Francisco © 1994); QUANTA/CHARMM (Molecular Simulations, Inc., Burlington, Mass., ©1994)). Specialized computer programs may also assist in the process of selecting fragments or chemical groups. These include: GRID (Goodford, 1985, J. Med. Chem. 28:849-857), available from Oxford University, Oxford, UK; MCSS (Miranker & Karplus, 1991, Proteins: Structure, Function and Genetics 11:29-34), available from Molecular Simulations, Burlington, Mass.; AUTODOCK (Goodsell & Olsen, 1990, Proteins: Structure, Function, and Genetics 8:195-202), available from Scripps Research Institute, La Jolla, Calif.; and DOCK (Kuntz et al., 1982, J. Mol. Biol. 161:269-288), available from University of California, San Francisco, Calif. Once suitable chemical groups or fragments that bind to LOX-1 have been selected, they can be assembled into a single compound or inhibitor. Assembly may proceed by visual inspection of the relationship of the fragments to each other in the three-dimensional image displayed on a computer screen in relation to the structure coordinates thereof. This would be followed by manual model building using software such as QUANTA or SYBYL. Useful programs to aid one of skill in the art in connecting the individual chemical groups or fragments include: CAVEAT (Bartlett et al., 1989, ‘CAVEAT: A Program to Facilitate the Structure-Derived Design of Biologically Active Molecules’. In Molecular Recognition in Chemical and Biological Problems', Special Pub., Royal Chem. Soc. 78:182-196), available from the University of California, Berkeley, Calif.; 3D Database systems such as MACCS-3D (MDL Information Systems, San Leandro, Calif.). This area is reviewed in Martin, 1992, J. Med. Chem. 35:2145-2154); and HOOK (available from Molecular Simulations, Burlington, Mass.). Instead of proceeding to build a LOX-1 binding partner that selectively inhibits the binding of one ligand but not one other ligand to LOX-1, in a step-wise fashion, one fragment or chemical group at a time, as described above, such compounds may be designed as a whole or ‘de novo’ using either an empty binding site or the surface of a protein that participates in protein/protein interactions or optionally including some portion(s) thereof. These methods include: LUDI (Bohm, 1992, J. Comp. Aid. Molec. Design 6:61-78), available from Molecular Simulations, Inc., San Diego, Calif.; LEGEND (Nishibata & ltai, 1991, Tetrahedron 47:8985), available from Molecular Simulations, Burlington, Mass.; and LeapFrog (available from Tripos, Inc., St. Louis, Mo.). Other molecular modeling techniques may also be employed in accordance with this invention. See, e.g., Cohen et al., 1990, J. Med. Chem. 33:883-894. See also, Navia & Murcko, 1992, Current Opinions in Structural Biology 2:202-210.

Once a candidate binding partner has been designed by the above methods, the efficiency with which that binding partner may bind to or interact with LOX-1 may be tested and optimized by computational evaluation. Selective binding partners may interact with the receptor in more than one conformation that is similar in overall binding energy. In those cases, the deformation energy of binding is taken to be the difference between the energy of the free compound and the average energy of the conformations observed when the binding partner binds to the receptor protein.

Specific computer software is available in the art to evaluate compound deformation energy and electrostatic interaction. Examples of programs designed for such uses include: Gaussian 92, revision C (Frisch, Gaussian, Inc., Pittsburgh, Pa. ©1992); AMBER, version 4.0 (Kollman, University of California at San Francisco © 1994); QUANTA/CHARMM (Molecular Simulations, Inc., Burlington, Mass., ©1994); and Insight Il/Discover (Biosym Technologies Inc., San Diego, Calif., © 1994). These programs may be implemented, for instance, using a computer workstation, as are well-known in the art. Other hardware systems and software packages will be known to those skilled in the art.

Once a LOX-1 binding partner (preferably a selective inhibitor of one ligand binding to LOX-1 but not one other ligand binding to LOX-1) has been optimally designed, for example as described above, substitutions may then be made in some of its atoms or chemical groups in order to improve or modify its binding properties, or its pharmaceutical properties such as stability or toxicity. Generally, initial substitutions are conservative, i.e., the replacement group will have approximately the same size, shape, hydrophobicity and charge as the original group. One of skill in the art will understand that substitutions known in the art to alter conformation should be avoided. Such altered chemical compounds may then be analyzed for efficiency of binding to LOX-1 by the same computer methods described in detail above.

A candidate binding partner or a candidate inhibitor refers to a composition which is evaluated in a test or assay, for example, to assess the ability to selectively inhibit the binding of one ligand to LOX-1, but not one other ligand for LOX-1. Examples of candidate binding partners include, but are not limited to, proteins, polypeptides, antibodies, small molecules and other drugs. In one embodiment, candidate binding partners can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683). Phage display libraries may be used to screen potential LOX-1 binding partners that may be further screened using the methods described herein for selectively inhibiting the binding of one ligand but not one other ligand for LOX-1. Their usefulness lies in the ability to screen, for example, a library displaying a billion different compounds with only a modest investment of time, money, and resources. For use of phage display libraries in a screening process, see, for instance, Kay et al., Methods, 240-246, 2001.

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

The methods of screening candidate binding partners may also include the specific identification or characterization of such binding partners, whose LOX-1 binding potential was determined by the methods described above. If the identity of the compound is known from the start of the experiment, no additional assays are needed to determine its identity. However, if the screening for compounds that bind LOX-1 is done with a library of compounds, it may be necessary to perform additional tests to positively identify a compound that satisfies all required conditions of the screening process. There are multiple ways to determine the identity of the compound. One process involves mass spectrometry, for which various methods are available and known to the skilled atisan (see for instance neogenesis.com). Neogenesis' ALIS (au toma ted ligand identification system) spectral search engine and data analysis software allow for a highly specific identification of a binding partner structure based on the exact mass of the binding partner. One skilled in the art can also readily perform mass spectrometry experiments to determine the identity of the compound.

Antibodies, including polyclonal and monoclonal antibodies, particularly anti-LOX-1 antibodies may be useful as binding partners that bind to LOX-1 and selectively inhibit the binding of one ligand but not one other ligand to LOX-1. While certain anti-LOX-1 antibodies are commercially available, these do not provide the selectivity described above. However, the present invention provides novel anti-LOX-1 antibodies that provide such selectivity, as described herein. These antibodies may be generated or prepared using standard procedures for preparation of polyclonal or monoclonal antibodies known to those skilled in the art. Also, these antibodies, including both polyclonal and monoclonal antibodies, may possess certain diagnostic applications and may for example, be utilized for the purpose of detecting and/or measuring conditions such as atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation.

LOX-1 may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the activity(ies) of LOX-1 may be discovered or synthesized, and may be used in diagnostic and/or therapeutic protocols.

Proteins or polypeptides for use as binding partners, as described herein, optionally, further include a moiety that enhances one or more of, e.g., stability, effector cell function or complement fixation. For example, an antibody or antigen-binding protein can further include a pegylated moiety, or albumin.

As noted above, the present invention provides assays for identifying candidate binding partners e.g. anti-LOX-1 antibodies or fragments thereof, that selectively inhibit the binding of one LOX-1 ligand to LOX-1, but not one other LOX-1 ligand (e.g., oxLDL or CRP) to a LOX-1 receptor.

In one embodiment, the assays detect candidate binding partners, e.g. anti-LOX-1 antibodies that modulate the signaling activities of the LOX-1 receptor induced by ligand binding to LOX-1, wherein the ligand may be selected from the group consisting of a modified lipoprotein, such as oxLDL or AcLDL, an anionic phospholipid, a cellular ligand, a bile salt-dependent lipase and C-reactive protein. Such signaling activities include, but are not limited to, binding to other cellular components, activating NF-κB transcriptional activity, and the like.

The above-noted LOX-1 ligands are relevant to signaling pathways involved in inflammatory conditions, as well as, cell growth and proliferation, including cancerous cell growth.

A variety of assay formats will suffice and, in light of the present disclosure, those not expressly described herein will nevertheless be comprehended by one of ordinary skill in the art. Assay formats, which approximate such conditions as formation of protein complexes, enzymatic activity, may be generated in many different forms, and include assays based on cell-free systems, e.g., purified proteins or cell lysates, as well as cell-based assays which utilize intact cells. Simple binding assays can be used to detect compounds that inhibit the interaction between a LOX-1 ligand (e.g a modified lipoprotein, an anionic phospholipid, a cellular ligand, a bile salt-dependent lipase and C-reactive protein) and LOX-1. Compounds to be tested can be produced, for example, by bacteria, yeast or other organisms (e.g., natural products), produced chemically (e.g., small molecules, including peptidomimetics), or produced recombinantly.

In one embodiment, a cell is manipulated after incubation with a candidate binding partner and assayed for signaling activities of the LOX-1 receptor induced by binding of the receptor with one of the ligands described above. In one embodiment, a bioassay for such activities may include NF-κB activity assays (e.g., NF-κB luciferase or GFP reporter gene assays).

Exemplary NF-κB luciferase or GFP reporter gene assays may be carried out as described by McFarlane et al. (McFarlane, S. M. et al. (2002) FEBS Letters. 515: 119-126). Briefly, cells expressing LOX-1 or a variant thereof are transfected with an NF-κB-luciferase reporter gene. The transfected cells are then incubated with a known ligand for LOX-1 and with or without a candidate binding partner. Subsequently, NF-κB-stimulated luciferase activity is measured in cells treated with the candidate binding partner or without the candidate binding partner. Alternatively, cells can be transfected with an NF-κB-GFP reporter gene (Stratagene). The transfected cells are then incubated with a known ligand for LOX-1 and with or without a candidate binding partner. Subsequently, NF-κB-stimulated gene activity are monitored by measuring GFP expression with a fluorescence/visible light microscope set-up or by FACS analysis.

In one embodiment, the present invention provides a method for reconstituting a protein so preparations include a receptor polypeptide (e.g., LOX-1), and one or more LOX-1 ligands. Assays of the present invention include labeled in vitro protein-protein binding assays, immunoassays for protein binding, and the like. The purified LOX-1 protein may also be used for determination of three-dimensional crystal structure, which can be used for modeling intermolecular interactions. The purified LOX-1 antibody may also be used for determination of three-dimensional crystal structure, which can be used for modeling intermolecular interactions.

In one embodiment, a LOX-1 ligand or a LOX-1 receptor polypeptide (e.g., LOX-1) can be endogenous to the cell selected to support the assays. Alternatively, a LOX-1 ligand or a LOX-1 receptor polypeptide (e.g., LOX-1) can be derived from exogenous sources. For instance, polypeptides can be introduced into the cell by recombinant techniques (such as through the use of an expression vector), as well as by microinjecting the polypeptide itself or mRNA encoding the polypeptide.

In one embodiment, a complex between a LOX-1 ligand and a LOX-1 receptor polypeptide can be generated in whole cells, taking advantage of cell culture techniques to support the subject assays. For example, as described below, a complex can be constituted in a eukaryotic cell culture system, including mammalian and yeast cells. Advantages to generating the subject assays in an intact cell include the ability to detect compounds that are functional in an environment more closely analogous to that for therapeutic use of the compounds. Furthermore, certain of the in vivo embodiments of the assay, such as examples given below, are amenable to high through-put analysis of candidate binding partners.

In one in vitro embodiment of the present assay, a reconstituted complex comprises a reconstituted mixture of at least semi-purified proteins. By semi-purified, it is meant that the proteins utilized in the reconstituted mixture have been previously separated from other cellular proteins. For instance, in contrast to cell lysates, proteins involved in the complex formation are present in the mixture to at least 50% purity relative to all other proteins in the mixture, in one embodiment are present at 90-95% purity, and in a further embodiment are present at 95-99% purity. In one embodiment of the subject method, the reconstituted protein mixture is derived by mixing highly purified proteins such that the reconstituted mixture substantially lacks other proteins (such as of cellular origin) that might interfere with or otherwise alter the ability to measure the complex assembly and/or disassembly.

In one embodiment, assaying in the presence and absence of a candidate binding partner can be accomplished in any vessel suitable for containing the reactants. Examples include microtitre plates, test tubes and micro-centrifuge tubes.

In one embodiment, drug screening assays can be generated which detect candidate binding partners, e.g. anti-LOX-1 antibodies, on the basis of their ability to interfere with assembly, stability or function of a complex between a LOX-1 ligand (e.g., oxLDL or CRP) and a LOX-1 receptor polypeptide (e.g., LOX-1). In an exemplary binding assay, the compound of interest is contacted with a mixture comprising a LOX-1 ligand and a LOX-1 receptor. Detection and quantification of the complex provide a means for determining the candidate binding partner's efficacy at inhibiting interaction between the two components of the complex. The efficacy of the candidate binding partner can be assessed by generating dose response curves from data obtained using various concentrations of the test antibody. Moreover, a control assay can also be performed to provide a baseline for comparison. In the control assay, the formation of complexes is quantitated in the absence of the candidate binding partner.

In one embodiment, association between the two polypeptides in a complex (e.g., a LOX-1 ligand and a LOX-1 receptor polypeptide), may be detected by a variety of techniques, many of which are effectively described above. For instance, modulation in the formation of complexes can be quantitated using, for example, detectably labeled proteins (e.g., radiolabeled, fluorescently labeled, or enzymatically labeled), by immunoassay, by two-hybrid assay, or by chromatographic detection. Surface plasmon resonance systems, such as those available from Biacore International AB (Uppsala, Sweden), may also be used to detect protein-protein interaction.

In one embodiment, one polypeptide in a complex comprising a LOX-1 ligand and a LOX-1 receptor polypeptide can be immobilized to facilitate separation of the complex from uncomplexed forms of the other polypeptide, as well as to accommodate automation of the assay. In one embodiment, an antibody can be provided which adds a domain that permits the antibody to be bound to an insoluble matrix. For example, an antibody can be absorbed onto glutathione sepharose beads (Sigma Chemical, St. Louis, Mo.) or glutathione derivatized microtitre plates, or directly or indirectly attached to magnetic beads, which are then combined with a potential interacting protein (e.g., an ³⁵S-labeled ligand), and the test antibody are incubated under conditions conducive to complex formation. Following incubation, the beads are washed to remove any unbound interacting antibody, and the matrix bead-bound radiolabel determined directly (e.g., beads placed in scintillant), or in the supernatant after the complexes are dissociated, e.g., when microtitre plate is used. Alternatively, after washing away unbound antibody, the complexes can be dissociated from the matrix, separated by SDS-PAGE gel, and the level of interacting polypeptide found in the matrix-bound fraction quantitated from the gel using standard electrophoretic techniques.

In one embodiment, ALPHAscreen technology may be used to screen for receptor ligand interactions and for determining the effect of candidate binding partners on the interaction between the receptor and its ligand(s). This technology was first described by Ullman in 1994 (Ullman, E F et al., Proc. Nat. Acad. Sci. USA, 1994, 91:5426-5430) and it is based on the principle of luminescent oxygen channeling ((Ullman, E F, et al., Clin. Chem. 1996, 42: 1518-1526; UlIman, E F et al., Proc. Nat. Acad. Sci. USA, 1994, 91:5426-5430). The ALPHA screen, which is now commercially available (Perkin Elmer Alphascreen® Histidine detection kit, 676069M), is a bead based, nonradioactive amplified luminescent proximity homogenous assay, in which a donor and an acceptor pair of 250 nm diameter reagent-coated polystyrene microbeads are brought into proximity by a biomolecular interaction of binding partners immobilized to the beads. Excitation of the assay mixture with a high intensity laser at 680 nm induces the formation of singlet oxygen at the surface of the donor bead, following conversion of ambient oxygen to a more excited singlet state by a photosensitizer present in the donor bead. The singlet oxygen molecules can diffuse up to 200 nm, and, if an acceptor bead is in the proximity, can react with a thioxene derivative present in the bead, generating chemiluminescence at 370 nm that further activates the fluorophores contained in the same bead. The fluorophores subsequently emit light at 520-620 nm. The donor bead generates about 60,000 singlet oxygen molecules, resulting in an amplified signal.

In one embodiment, a two-hybrid assay (also referred to as an interaction trap assay) can be used for detecting the interaction of two polypeptides in the complex of LOX-1 and LOX-1 ligand (see also, U.S. Pat. No. 5,283,317; Zervos et al. (1993) Cell 72: 223-232; Madura et al. (1993) J Biol Chem 268: 12046-12054; Bartel et al. (1993) Biotechniques 14: 920-924; and Iwabuchi et al. (1993) Oncogene 8: 1693-1696), and for subsequently detecting candidate binding partners, e.g. anti-LOX-1 antibodies which inhibit binding between a LOX-1 and a LOX-1 ligand. This assay includes providing a host cell, for example, a yeast cell (preferred), a mammalian cell or a bacterial cell type. The host cell contains a reporter gene having a binding site for the DNA-binding domain of a transcriptional activator used in the bait protein, such that the reporter gene expresses a detectable gene product when the gene is transcriptionally activated. A first chimeric gene is provided which is capable of being expressed in the host cell, and encodes a “bait” polypeptide. A second chimeric gene is also provided which is capable of being expressed in the host cell, and encodes the “fish” polypeptide. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

In one embodiment, the invention provides a two-hybrid assay to identify candidate binding partners, e.g. anti-LOX-1 antibodies that inhibit the binding of a LOX-1 ligand (e.g., oxLDL or CRP and a receptor polypeptide (e.g., LOX-1). To illustrate, a “bait” polypeptide comprising a receptor polypeptide and a “fish” polypeptide comprising a LOX-1 ligand (such as oxLDL or CRP), are introduced in the host cell. In one embodiment, the bait comprises human or murine LOX-1, or a sequence with 80 to 99% identity to human or murine LOX-1 that can bind LOX-1-ligand. Cells are subjected to conditions under which the bait and fish polypeptides are expressed in sufficient quantity for the reporter gene to be activated.

The interaction of the two fusion polypeptides results in a detectable signal produced by the expression of the reporter gene. Accordingly, the level of interaction between the two polypeptides in the presence of a candidate binding partner, e.g a test anti-LOX-1 antibody, and in the absence of the test anti-LOX-1 antibody can be evaluated by detecting the level of expression of the reporter gene in each case. Various reporter constructs may be used in accord with the methods of the invention and include, for example, reporter genes which produce such detectable signals as selected front the group consisting of an enzymatic signal, a fluorescent signal, a phosphorescent signal and drug resistance.

In many drug screening programs that test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test antibody. Moreover, the effects of cellular toxicity and/or bioavailability of the test antibody can be generally ignored in the in vitro system, the assay instead being focused primarily on the effect of the drug on the molecular target as may be manifest in an alteration of binding affinity with other proteins or changes in enzymatic properties of the molecular target.

In one embodiment, a complex formation between a LOX-1 ligand and a LOX-1 receptor, and the effect of a candidate binding partner on complex formation, may be assessed by immunoprecipitation and analysis of co-immunoprecipitated proteins or affinity purification and analysis of co-purified proteins. Fluorescence Resonance Energy Transfer (FRET)-based assays may also be used to determine such complex formation. The occurrence of FRET also causes the fluorescence lifetime of the donor fluorescent moiety to decrease. This change in fluorescence lifetime can be measured using a technique termed fluorescence lifetime imaging technology (FLIM) (Verveer et al. (2000) Science 290: 1567-1570, Squire et al. (1999) J: Microsc. 193: 36; Verveer et al. (2000) Biophys. J. 78: 2127). Global analysis techniques for analyzing FLIM data have been developed. These algorithms use the understanding that the donor fluorescent moiety exists in only a limited number of states each with a distinct fluorescence lifetime. Quantitative maps of each state can be generated on a pixel-by-pixel basis.

To perform FRET-based assays, a LOX-1 ligand (e.g., oxLDL or CRP) and a LOX-1 receptor polypeptide (e.g., LOX-1) are both fluorescently labeled. Suitable fluorescent labels are well known in the art. Examples are provided below, but suitable fluorescent labels not specifically discussed are also available to those of skill in the art and may be used. Fluorescent labeling may be accomplished by expressing a polypeptide as a polypeptide with a fluorescent protein, for example fluorescent proteins isolated from jellyfish, corals and other coelenterates. Exemplary fluorescent proteins include the many variants of the green fluorescent protein (GFP) of Aequoria victoria. Variants may be brighter, dimmer, or have different excitation and/or emission spectra. Certain variants are altered such that they no longer appear green, and may appear blue, cyan, yellow or red (termed BFP, CFP, YFP, and REP, respectively). Fluorescent proteins may be stably attached to polypeptides through a variety of covalent and noncovalent linkages, including, for example, peptide bonds (e.g., expression as a fusion protein), chemical cross-linking and biotin-streptavidin coupling. For examples of fluorescent proteins, see U.S. Pat. Nos. 5,625,048, 5,777,079, 6,066,476, and 6,124,128, Prasher et al. (1992) Gene, 111: 229-233; Reign et al. (1994) Proc. Natl. Acad. Sci., USA, 91: 12501-04; Ward et al. (1982) Photochem. Photobiol., 35: 803-808; Levine et al. (1982) Comp. Biochem. Physiol., 72B: 77-g5; Tersikh et al. (2000) Science 290: 1585-88.

FRET-based assays may be used in cell-based assays and in cell-free assays. FRET-based assays are amenable to high-throughput screening methods including Fluorescence Activated Cell Sorting and fluorescent scanning of microtiter arrays.

In general, where a screening assay is a binding assay (whether protein-protein binding, compound-protein binding, etc.), one or more of the molecules may be coupled or linked to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g., albumin, detergents, etc that are used to facilitate optimal protein-protein binding and/or reduce nonspecific or battleground interactions. Reagents that improve the efficiency of the assay, such as protease inhibitors, nuclease inhibitors, anti-microbial compounds, etc. may be used. The mixture of components are added in any order that provides for the requisite binding. Incubations are performed at any suitable temperature, typically between 4° C. and 40° C. Incubation periods are selected for optimum activity, but may also be optimized to facilitate rapid high-throughput screening.

In one embodiment, the invention provides complex-independent assays. Such assays comprise identifying a candidate binding partner, e.g a test antibody that is a candidate inhibitor of the binding of one LOX-1 ligand to LOX-1, but not one other LOX-1 ligand to LOX-1.

In one embodiment, a compound that binds to a LOX-1 receptor polypeptide may be identified by using a LOX-1 receptor to which has been added an additional domain that permits the protein to be bound to an insoluble matrix. For example, a LOX-1 polypeptide fused with a GST protein can be adsorbed onto glutathione sepharose beads (Sigma Chemical, St. Louis, Mo.) or glutathione derivatized microtitre plates, which are then combined with a potential labeled binding compound and incubated under conditions conducive to binding. Following incubation, the beads are washed to remove any unbound compound, and the matrix bead-bound label determined directly, or in the supernatant after the bound compound is dissociated.

In one embodiment, a label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures. In one embodiment, such methods comprise forming the mixture in vitro. In one embodiment, such methods comprise cell-based assays by forming the mixture in vivo. In one embodiment, the methods comprise contacting a cell that expresses a receptor polypeptide (e.g., LOX-1) or a variant thereof with the candidate binding partner, e.g a test antibody.

In one embodiment, an assay is based on a cell-free system, e.g., purified proteins or cell lysates, as well as a cell-based assay that utilizes intact cells. Simple binding assays can be used to detect compounds that interact with the receptor polypeptide. Compounds to be tested can be produced, for example, by bacteria, yeast or other organisms (e.g., natural products), produced chemically (e.g., small molecules, including peptidomimetics), or produced recombinantly.

Optionally, candidate binding partners, e.g. anti-LOX-1 antibodies identified from these assays may be used to treat or diagnose LOX-1-associated disorders.

One embodiment of the invention provides a method of using a binding partner of LOX-1, e.g. an antibody against LOX-1, or nucleic acids encoding LOX-1 to diagnose a subject having or predisposed to having, a disease characterized by high levels of LOX-1 and/or a LOX-1 ligand such as atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation. Thus, in another embodiment of the invention, one may look for a decrease in expression of the LOX-1 gene or gene product after appropriate therapy for these conditions.

The diagnostic method of the invention provides contacting a biological sample such as a biopsy sample, tissue, or cell isolated from a subject with an antibody which binds LOX-1. The antibody is allowed to bind to the LOX-1 antigen to form an antibody-antigen complex. The LOX-1 antigen, as used herein, includes the LOX-1 protein or peptides isolated therefrom. The conditions and time required to form the antibody-antigen complex may vary and are dependent on the biological sample being tested and the method of detection being used. Once non-specific interactions are removed by, for example, washing the sample, the antibody-antigen complex is detected using any immunoassay used to detect and/or quantitate antigens [see, for example, Harlow and Lane, Antibodies: A Laboratory Manual, Cold Spring Harbor Laboratory, New York (1988) 555-612]. Such well-known immunoassays include antibody capture assays, antigen capture assays, and two-antibody sandwich assays. In an antibody capture assay, the antigen is attached to solid support, and labeled antibody is allowed to bind. After washing, the assay is quantitated by measuring the amount of antibody retained on the solid support. In an antigen capture assay, the antibody is attached to a solid support, and labeled antigen is allowed to bind. The unbound proteins are removed by washing, and the assay is quantitated by measuring the amount of antigen that is bound. In a two-antibody sandwich assay, one antibody is bound to a solid support, and the antigen is allowed to bind to this first antibody. The assay is quantitated by measuring the amount of a labeled second antibody that binds to the antigen.

These immunoassays typically rely on labeled antigens, antibodies, or secondary reagents for detection. These proteins may be labeled with radioactive compounds, enzymes, biotin, or fluorochromes. Of these, radioactive labeling may be used for almost all types of assays. Enzyme-conjugated labels are particularly useful when radioactivity must be avoided or when quick results are needed. Biotin-coupled reagents usually are detected with labeled streptavidin. Streptavidin binds tightly and quickly to biotin and may be labeled with radioisotopes or enzymes. Fluorochromes, although requiring expensive equipment for their use, provide a very sensitive method of detection. Those of ordinary skill in the art will know of other suitable labels, which may be employed in accordance with the present invention. The binding of these labels to antibodies or fragments thereof may be accomplished using standard techniques such as those described by Kennedy, et al. [(1976) Clin. Chim. Acta 70:1-31], and Schurs, et al. [(1977) Clin. Chim Acta 81:1-40].

In accordance with the diagnostic methods of the invention, the presence or absence of the antibody-antigen complex is correlated with the presence or absence in the biological sample of the LOX-1 gene product. A biological sample containing elevated levels of the LOX-1 gene product is indicative of atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation. Accordingly, the diagnostic methods of the invention may be used as part of a routine screen in subjects suspected of having such diseases or for subjects who may be predisposed to having such diseases. Moreover, the diagnostic methods of the invention may be used alone or in combination with other well-known diagnostic methods to confirm such diseases.

The diagnostic methods of the invention further provide that an antibody of the invention may be used to monitor the levels of LOX-1 antigen in patient samples at various intervals of drug treatment to identify whether and to which degree the drug treatment is effective in restoring health. Furthermore, LOX-1 antigen levels may be monitored using an antibody of the invention in studies evaluating efficacy of drug candidates in model systems and in clinical trials. For example, using an antibody of this invention, LOX-1 antigen levels may be monitored in biological samples of individuals treated with known or unknown therapeutic agents. This may be accomplished with cell lines in vitro or in model systems and clinical trials, depending disease being investigated. Increased total levels of LOX-1 antigen in biological samples during or immediately after treatment with a drug candidate indicates that the drug candidate may actually exacerbate the disease. No change in total levels of LOX-1 antigen indicates that the drug candidate is ineffective in treating the disease. A lowering in total levels of LOX-1 antigen indicates that the drug candidate is effective in treating the disease. This may provide valuable information at all stages of pre-clinical drug development, clinical drug trials as well as subsequent monitoring of patients undergoing drug treatment.

Variants of anti-LOX-1 antibodies of the invention, which act as binding partners that selectively inhibit the binding of one ligand but not one other ligand for LOX-1, may be produced using standard recombinant techniques, including site-directed mutagenesis, or recombination cloning. A diversified repertoire of anti-LOX-1 antibodies may be prepared via gene arrangement and gene conversion methods in transgenic non-human animals (U.S. Patent Publication No. 2003/0017534), which are then tested for relevant activities using binding assays and functional assays. Variants may be obtained using an affinity maturation protocol for mutating CDRs (Yang et al. (1995) J. Mol. Biol. 254: 392-403), chain shuffling (Marks et al. (1992) Biotechnology (NY) 10: 779-783), use of mutator strains of E. coli (Low et al. (1996) J. Mol. Biol. 260: 359-368), DNA shuffling (Patten et al. (1997) Curr. Opin. Biotechnol. 8: 724-733), phage display (Thompson et al. (1996) J. Mol. Biol. 256: 77-88), and sexual PCR (Crameri et al. (1998) Nature 391: 288-291). Antibodies may also be produced in transgenic animals (Houdebine (2002) Curr. Opin. Biotechnol. 13(6):625-629) and transgenic plants (Schillberg et al. (2003) Cell Mol. Life Sci. 60(3):433-45).

As discussed above, binding partners that demonstrate selectivity for one ligand of LOX-1, but not one other ligand of LOX-1, may be monoclonal, chimeric and humanized antibodies, which have been modified by, e.g., deleting, adding, or substituting other portions of the antibody, e.g., the constant region, are also within the scope of the invention. For example, an antibody can be modified as follows: (i) by deleting the constant region; (ii) by replacing the constant region with another constant region, e.g., a constant region meant to increase half-life, stability or affinity of the antibody, or a constant region from another species or antibody class; or (iii) by modifying one or more amino acids in the constant region to alter, for example, the number of glycosylation sites, effector cell function, Fc receptor (FcR) binding, complement fixation, among others.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260, the contents of all of which are hereby incorporated by reference). Similar type of alterations could be described which if applied to the murine, or other species immunoglobulin would reduce or eliminate these functions.

For example, it is possible to alter the affinity of an Fc region of an antibody (e.g., an IgG, such as a human IgG) for an FcR (e.g., FcγR1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

In one embodiment, the binding partner that is an antibody of the present invention can be administered in combination with other agents as part of a combinatorial therapy. For example, in the case of inflammatory conditions, the subject antibodies can be administered in combination with one or more other agents useful in the treatment of inflammatory diseases or conditions. In the case of cardiovascular disease conditions, and particularly those arising from atherosclerotic plaques, which are thought to have a substantial inflammatory component, the subject antibodies can be administered in combination with one or more other agents useful in the treatment of cardiovascular diseases. In the case of cancer, the subject antibodies can be administered in combination with one or more anti-angiogenic factors, chemotherapeutics, or as an adjuvant to radiotherapy. It is further envisioned that the administration of the subject antibodies will serve as part of a cancer treatment regimen that may combine many different cancer therapeutic agents. In the case of IBD, the subject antibodies can be administered with one or more anti-inflammatory agents, and may additionally be combined with a modified dietary regimen.

The invention includes methods for selectively inhibiting the interaction between LOX-1 and one LOX-1 ligand, but not one other LOX-1 ligand. Preferably, such methods are used for treating LOX-1-associated disorders.

Such methods may comprise administering a binding partner for LOX-1, for example, an antibody raised to LOX-1 as disclosed herein. Such methods comprise administering an antibody that binds specifically to one or more epitopes of a LOX-1 protein. In yet another embodiment, such methods comprise administering a compound that inhibits the binding of LOX-1 to one LOX-1 ligand, but not to one other ligand. In one embodiment, the LOX-1 ligand is oxidized LDL. In one embodiment, the LOX-1 ligand is C reactive protein. Exemplary methods of identifying such compounds were discussed previously.

In one embodiment, the interaction is inhibited in vitro, such as in a reaction mixture comprising purified proteins, cells, biological samples, tissues, artificial tissues, etc. In one embodiment, the interaction is inhibited in vivo, for example, by administering an antibody that binds to LOX-1 or a LOX-1-binding fragment thereof. The antibody or fragment thereof bind to LOX-1 and inhibit binding of one LOX-1 ligand, but not one other LOX-1 ligand.

The invention includes methods for preventing or treating a LOX-1 related disorder by inhibiting the interaction between LOX-1 and a LOX-1 ligand. Such methods include administering an antibody to LOX-1 in an amount effective to inhibit the interaction and for a time sufficient to prevent or treat said disorder.

Representative nucleic acids of the invention comprise nucleotide sequences encoding the variable heavy and variable light chains of two rat anti-mouse LOX-1 antibodies, designated LOX1-A11A2 and LOX1-33F1. The sequences described below do not contain the signal sequences. Nucleic acids are deoxyribonucleotides or ribonucleotides and polymers thereof in single-stranded, double-stranded, or triplexed form. Unless specifically limited, nucleic acids may contain known analogues of natural nucleotides that have similar properties as the reference natural nucleic acid. Nucleic acids include genes, cDNAs, mRNAs, and cRNAs. Nucleic acids may be synthesized, or may be derived from any biological source, including any organism.

SEQ ID NO. 4 is the nucleotide sequence encoding the variable heavy chain of the antibody designated LOX1-11A2 and SEQ ID NO: 2 is the nucleotide sequence encoding the variable light chain of the antibody designated LOX1-11A2. The corresponding amino acid sequences of the variable heavy and variable light chain of anti-LOX-1 antibody LOX1-11A2 are shown in SEQ ID NOs: 3 and 1, respectively.

SEQ ID NO. 8 is the nucleotide sequence encoding the variable heavy chain of the antibody designated LOX1-33F1 and SEQ ID NO: 6 is the nucleotide sequence encoding the variable light chain of the antibody designated LOX1-33F1. The corresponding amino acid sequences of the variable heavy and variable light chain of anti-LOX-1 antibody LOX1-33F1 are shown in SEQ ID NOs. 7 and 5, respectively.

Nucleic acids of the invention may also comprise a nucleotide sequence that is substantially identical to any one of SEQ ID NOs: 2, 4, 6 and 8, including nucleotide sequences that are at least 90%, 91%, 92%, 93%, 94%, 95%, 96%, 97%, 98%, 99%, 99.5%, or 99.9% identical to any one of SEQ ID NOs: 2, 4, 6 and 8.

Nucleic acids of the invention may also comprise a nucleotide sequence encoding an anti-LOX-1 antibody variable region having an amino acid sequence that is substantially identical to any of the amino acid sequences shown in SEQ ID NOs: 1, 3, 5 and 7, including a nucleotide sequence encoding an amino acid sequence that is at least 85%, 86%, 87%, 88%, 89%, 90%, 91%, 92%, 93%, 94%, 95%, 96%, 97%, 98%, 99%, 99.5%, or 99.9% identical to any of SEQ ID NOs: 1, 3, 5 and 7.

Sequences are compared for maximum correspondence using a sequence comparison algorithm using the full-length variable region encoding sequence of any one of SEQ ID NOs: 1, 3, 5 and 7, a nucleotide sequence encoding a full length variable region having any one of the sequences shown in SEQ ID NO: 1, 3, 5 and 7 as the query sequence, as described herein below, or by visual inspection.

Substantially identical sequences may be polymorphic sequences, i.e., alternative sequences or alleles in a population. An allelic difference may be as small as one base pair. Substantially identical sequences may also comprise mutagenized sequences, including sequences comprising silent mutations. A mutation may comprise one or more residue changes, a deletion of one or more residues, or an insertion of one or more additional residues.

Substantially identical nucleic acids are also identified as nucleic acids that hybridize specifically to or hybridize substantially to a nucleotide sequence encoding an antibody variable heavy chain or antibody variable light chain as shown in any one of SEQ ID NOs: 2, 4, 6 and 8, under stringent conditions. In the context of nucleic acid hybridization, two nucleic acid sequences being compared may be designated a probe and a target. A probe is a reference nucleic acid molecule, and a target is a test nucleic acid molecule, often found within a heterogeneous population of nucleic acid molecules. A target sequence is synonymous with a test sequence.

For hybridization studies, useful probes are complementary to or mimic at least about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of any one of SEQ ID NOs: 2, 4, 6, and 8, or to the full length of any nucleotide sequence encoding a variable heavy or variable light chain amino acid sequence of a LOX-1 antibody as shown in SEQ ID NOs: 2, 4, 6 and 8. Such fragments may be readily prepared, for example, by chemical synthesis of the fragment, by application of nucleic acid amplification technology, or by introducing selected sequences into recombinant vectors for recombinant production.

Specific hybridization refers to the binding, duplexing, or hybridizing of a molecule only to a particular nucleotide sequence under stringent conditions when that sequence is present in a complex nucleic acid mixture (e.g., total cellular DNA or RNA). Specific hybridization may accommodate mismatches between the probe and the target sequence depending on the stringency of the hybridization conditions.

Stringent hybridization conditions and stringent hybridization wash conditions in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, part I chapter 2, Elsevier, New York, N.Y. Generally, highly stringent hybridization and wash conditions are selected to be about 5° C. lower than the thermal melting point (Tm) for the specific sequence at a defined ionic strength and pH. Typically, under stringent conditions a probe will hybridize specifically to its target subsequence, but to no other sequences.

The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds (1989) Molecular Cloning: A Laboratory Manual, Cold Spring Harbor Laboratory Press, Cold Spring Harbor, N.Y., for a description of SSC buffer. Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4×. to 6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions may also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that may be used to identify nucleotide sequences that are substantially identical to reference nucleotide sequences encoding the LOX-1 binding partners, e.g. the variable heavy and variable light chain anti-LOX-1 antibodies, of the present invention: a probe nucleotide sequence preferably hybridizes to a target nucleotide sequence in 7% sodium dodecyl sulphate (SDS), 0.5M NaPO₄, 1 mM EDTA at 50° C. followed by washing in 2×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.5×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further herein below. Nucleic acid molecules that do not hybridize to each other under stringent conditions are still substantially identical if the corresponding proteins are substantially identical. This may occur, for example, when two nucleotide sequences comprise conservatively substituted variants as permitted by the genetic code.

Conservatively substituted variants are nucleic acid sequences having degenerate codon substitutions wherein the third position of one or more selected (or all) codons is substituted with mixed-base and/or deoxyinosine residues. See Batzer et al. (1991) Nucleic Acids Res. 19:5081; Ohtsuka et al. (1985) J. Biol. Chem. 260:2605-2608; and Rossolini et al. (1994) Mol. Cell Probes 8:91-98.

Nucleic acids of the invention also comprise nucleic acids complementary to any one of SEQ ID NOs: 2, 4, 6 and 8, or nucleotide sequences encoding an antibody variable region amino acid sequence shown in SEQ ID NOs: 2, 4, 6 and 8, and complementary sequences thereof. Complementary sequences are two nucleotide sequences that comprise antiparallel nucleotide sequences capable of pairing with one another upon formation of hydrogen bonds between base pairs. As used herein, the term complementary sequences means nucleotide sequences which are substantially complementary, as may be assessed by the same nucleotide comparison methods set forth below, or is defined as being capable of hybridizing to the nucleic acid segment in question under relatively stringent conditions such as those described herein. A particular example of a complementary nucleic acid segment is an antisense oligonucleotide.

A subsequence is a sequence of nucleic acids that comprises a part of a longer nucleic acid sequence. An exemplary subsequence is a probe, described herein above, or a primer. The term primer as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

An elongated sequence comprises additional nucleotides (or other analogous molecules) incorporated into the nucleic acid. For example, a polymerase (e.g., a DNA polymerase) may add sequences at the 3′ terminus of the nucleic acid molecule. In addition, the nucleotide sequence may be combined with other DNA sequences, such as promoters, promoter regions, enhancers, polyadenylation signals, intronic sequences, additional restriction enzyme sites, multiple cloning sites, and other coding segments. Thus, the invention also provides vectors comprising the disclosed nucleic acids, including vectors for recombinant expression, wherein a nucleic acid of the invention is operatively linked to a functional promoter. When operatively linked to a nucleic acid, a promoter is in functional combination with the nucleic acid such that the transcription of the nucleic acid is controlled and regulated by the promoter region. Vectors refer to nucleic acids capable of replication in a host cell, such as plasmids, cosmids, and viral vectors.

Nucleic acids of the present invention may be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions is also known in the art. See e.g., Sambrook et al. (eds.) (1989) Molecular Cloning: A Laboratory Manual. Cold Spring Harbor Laboratory Press, Cold Spring Harbor, N.Y.; Silhavy et al. (1984) Experiments with Gene Fusions. Cold Spring Harbor Laboratory Press, Cold Spring Harbor, N.Y.; Glover & Hames (1995) DNA Cloning: A Practical Approach, 2nd ed. IRL Press at Oxford University Press, Oxford/N.Y.; Ausubel (ed.) (1995) Short Protocols in Molecular Biology, 3rd ed. Wiley, New York. The nucleic acids of the invention may be used to generate LOX-1 binding partners, e.g. selective antibodies to LOX-1 using standard molecular biology techniques, or they may be used to identify new binding partners as described in the invention.

The invention relates to and includes methods of treating LOX-1 or LOX-1 ligand-related or associated disorders. LOX-1-related disorders may be characterized generally as including any disorder in which an affected cell exhibits elevated expression of LOX-1 or one or more LOX-1 ligands. LOX-1-related disorders may also be characterized as any disorder that is treatable (i.e., one or more symptoms may be eliminated or ameliorated) by a decrease in LOX-1 expression and/or function. For example, LOX-1 expression and/or function can be decreased by administration of an agent, eg a binding partner, that disrupts the interaction between LOX-1 and one LOX-1 ligand, such as an antibody to LOX-1.

The increased expression of LOX-1 is associated with several pathological states, such as atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation. LOX-1 ligands are produced in tissue affected with many inflammatory disorders, including arthritis (such as osteoarthritis). The enhanced expression of LOX-1 is thought to play a role in endothelial cell dysfunction that leads to vascular disease in diabetics.

The invention includes a method of treating inflammation and diseases or conditions characterized by activation of the inflammatory cytokine cascade in a subject, comprising administering an effective amount of a LOX-1 binding partner, such as an anti-LOX-1 antibody or a composition (e.g., pharmaceutical composition) comprising an anti-LOX-1 antibody. Also, studies the effect of a LOX-1 binding partner, such as an anti-LOX-1 antibody can be done in particular animal models to assess their effectiveness in therapy for these conditions. For example, studies can be done in an animal model for a delayed-type hypersensitivity response, for colitis in IL-10 null mice, for collagen-induced arthritis, and for experimental autoimmune encephalitis model. An inflammatory condition that is suitable for the methods of treatment described herein can be one in which the inflammatory cytokine cascade is activated.

The inflammatory cytokine cascade may cause a systemic reaction, as occurs with septic shock. The LOX-1 binding partners, e.g. anti-LOX-1 antibodies and/or LOX-1-binding fragments thereof of the invention can be used to treat sepsis, or septic shock. Sepsis is a systemic inflammatory response to infection, and is associated with organ dysfunction, hypoperfusion, or hypotension. In septic shock, a severe form of sepsis, hypotension is induced despite adequate fluid resuscitation. Sepsis has a complex physiology, defined by systemic inflammation and organ dysfunction, including abnormalities in body temperature; cardiovascular parameters and leukocyte count; elevated liver enzymes and altered cerebral function. The response in sepsis is to an infection or stimulus that becomes amplified and dysregulated. The murine CLP model of sepsis results in a polymicrobial infection, with abdominal abscess and bacteremia, and recreates the hemodynamic and metabolic phases observed in human disease. Studies may be conducted in a murine CLP model of sepsis to show that LOX-1 plays an important role in the pathogenesis of sepsis. Administration of an anti-LOX-1 antibody that binds specifically to LOX-1 at the time of surgery, as well as up to 36 hours after the surgery, may provide significant therapeutic protection to the mice, as evidenced by increased survival and improved pathology scores.

The inflammatory condition that is treated or prevented by the antibodies and methods of the invention may be mediated by a localized inflammatory cytokine cascade. Non-limiting examples of inflammatory conditions that can be usefully treated using anti-LOX-1 antibodies and/or fragments thereof and/or compositions of the present invention include, e.g., diseases involving the gastrointestinal tract and associated tissues (such as ileus, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, coeliac disease, hepatitis, Crohn's disease, enteritis, and Whipple's disease); systemic or local inflammatory diseases and conditions (such as asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, and sarcoidosis); diseases involving the respiratory system and associated tissues (such as bronchitis, emphysema, rhinitis, cystic fibrosis, pneumonitis, adult respiratory distress syndrome, pneumoultramicroscopicsilicovolcanoconiosis, alvealitis, bronchiolitis, pharyngitis, pleurisy, and sinusitis); diseases arising from infection by various viruses (such as influenza, respiratory syncytial virus, HIV, hepatitis B virus, hepatitis C virus and herpes), bacteria (such as disseminated bacteremia, Dengue fever), fingi (such as candidiasis) and protozoal and multicellular parasites (such as malaria, filariasis, amebiasis, and hydatid cysts); dermatological diseases; diseases involving the cardiovascular system and associated tissues (such as stenosis, restenosis, vasulitis, angiitis, endocarditis, arteritis, atherosclerosis, thrombophlebitis, pericarditis, congestive heart failure, myocarditis, myocardial ischemia, periarteritis nodosa, and rheumatic fever); diseases involving the central or peripheral nervous system and associated tissues (such as meningitis, encephalitis, multiple sclerosis, cerebral infarction, cerebral embolism, Guillane-Barre syndrome, neuritis, neuralgia, spinal cord injury, paralysis, and uveitis); diseases of the bones, joints, muscles and connective tissues (such as the various arthritides and arthralgias, osteomyelitis, fasciitis, Paget's disease, gout, periodontal disease, rheumatoid arthritis, and synovitis); other autoimmune and inflammatory disorders (such as myasthenia gravis, thryoiditis, systemic lupus erythematosus, Goodpasture's syndrome, Behcets's syndrome, allograft rejection, graft-versus-host disease, Type I diabetes, ankylosing spondylitis, Berger's disease, and Retier's syndrome); as well as various cancers, tumors and proliferative disorders (such as Hodgkins disease); and, in any case the inflammatory or immune host response to any primary disease.

Anti-LOX-1 antibodies or binding fragments thereof of the invention can be used to treat or prevent complications of diabetes, and pathological conditions associated with diabetes.

Anti-LOX-1 antibodies or binding fragments thereof of the invention can be used to treat or prevent atherosclerosis. It has been shown that oxLDL plays an important role in the pathogenesis of atherosclerosis (Kita, T. et al. Ann NY Acad Sci 2001, 947:199-205). As the major receptor for oxLDL, LOX-1 mediates most of the toxic effects of oxLDL. LOX-1 is also expressed in vivo in large arteries (aortic, carotid, thoracic, coronary arteries, and veins), which are the predilection sites of atherosclerosis. LOX-1 is expressed in macrophages, smooth muscle cells and vascular endothelial cells, which are the three most important cells involved in the development of atherosclerosis (Sawamura, T et al., Nature 1997, 386: 73-77; Shi, X et al. J cell Sci, 2001, 114:1273-1282). Upregulated LOX-1 expression was found in atherosclerotic lesions in humans and experimental animal models. In aorta without atherosclerosis, LOX-1 expression was undetectable, while in carotid arteries, endothelial cells covering early atherosclerotic lesions were more frequently positive for LOX-1 expression. In addition, the upregulation of LOX-1 mediated a series of pathophysiological effects in atherosclerosis. In particular, LOX-1 functions as a cell-adhesion molecule that mediates the platelet-endothelium interaction and is involved in endotoxin induced inflammation, which may initiate and promote atherosclerosis (Kakutani, M. Proc. Natl. Acad Sci USA 2000, 97:360-364). Also, some anti-atherosclerotic drugs, for example, statin drugs, could inhibit the oxLDL mediated LOX-1 expression, the uptake of oxLDL, the adhesion molecule expression and down-regulation of eNOS (Mehta, J L et al., Biochem Biophys Res Commun 2001, 289:857-861).

The binding partners of the invention, for example, the anti-LOX-1 antibodies, may be used to treat arthritis, such as, rheumatoid arthritis and osteoarthritis. In a rat zymosan-induced arthritis model, LOX-1 is expressed on synovial endothelium and postcapillary venules. In addition, administration of anti-LOX-1 antibody suppressed joint swelling, leukocyte infiltration and joint nitrite accumulation, as well as cartilage destruction, suggesting that LOX-1 might play a role in promoting joint inflammation and cartilage destruction. (Nakagawa, T. et al. Arthritis Rheum 2002, 46:2486-2494) Furthermore, Akagi et al found a significant increase in LOX-1 expression in human osteoarthritis samples and cultured chondrocytes and the increase in LOX-1 expression significantly correlated with the modified Mankin score. (Akagi, M et al. Osteoarthritis Cartilage 2006, 28 [Epub].

The binding partners of the invention, e.g. the anti-LOX-1 antibodies may also be used to treat ischemia-reperfusion and myocardial infarction. LOX-1 expression was observed in anesthetized rats subjected to myocardial ischemia-reperfusion. Treatment with LOX-1 antibody prevented ischemia-reperfusion induced upregulation of LOX-1, apoptosis, lipid peroxidation and reducing the myocardial infarct size induced by ischemia-reperfusion. (Li, D et al., J Am Coll Cardiol 2003, 41:1048-1055).

Accordingly, the list of LOX-1-related disorders that may be treated or prevented with an inventive composition include: acute inflammatory diseases (such as sepsis), shock (e.g., septic shock, hemorrhagic shock), chronic inflammatory diseases (such as rheumatoid and psoriatic arthritis, osteoarthritis, ulcerative colitis, irritable bowel disease, multiple sclerosis, psoriasis, lupus, systemic lupus nephritis, and inflammatory lupus nephritis, and other autoimmune diseases), cardiovascular diseases (e.g., atherosclerosis, stroke, fragile plaque disorder, angina and restenosis), diabetes (and particularly cardiovascular diseases in diabetics), complications of diabetes, cancers (e.g., lung cancer, squamous cell carcinoma, prostate cancer, human pancreatic cancer, renal cell carcinoma melanoma), vasculitis and other vasculitis syndromes such as necrotizing vasculitides, nephropathies, retinopathies, and neuropathies.

The invention provides for the administration of a binding partner to LOX-1, e.g. the anti-LOX-1 antibodies described herein, in vivo. The subject antibodies may be administered as pharmaceutical compositions, and may also be administered with one or more additional agents. The administration of the subject antibodies can be part of a therapeutic regimen to treat a particular condition. Conditions that can be treated by administration of either the antibodies alone, or by administration of the subject antibodies in combination with other agents, include LOX-1-associated disorders. By way of example, LOX-1-associated disorders include, but are not limited to, atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation, and other conditions that are aggravated by inflammation (i.e., the symptoms of which may be ameliorated by decreasing inflammation).

Methods of administration of the antibody based compositions can be by any of a number of methods well known in the art. These methods include local or systemic administration and further include intradermal, intramuscular, intraperitoneal, intravenous, subcutaneous, oral, and intranasal routes of administration, including use of a nebulizer and inhalation. In addition, it may be desirable to introduce the pharmaceutical compositions of the invention into the central nervous system by any suitable route, including intraventricular and intrathecal injection. Intraventricular injection may be facilitated by an intraventricular catheter, for example, attached to a reservoir, such as an Ommaya reservoir. Methods of introduction may also be provided by rechargeable or biodegradable devices, e.g., depots. Furthermore, it is contemplated that administration may occur by coating a device, implant, stent, or prosthetic.

For example, cartilage severely damaged by conditions of the joints such as rheumatoid arthritis and osteoarthritis can be replaced, in whole or in part, by various prosthetics. A variety of suitable transplantable materials exist including those based on collagen-glycosaminoglycan templates (Stone et al. (1990) Clin. Orthop. Relat. Red. 252: 129), isolated chondrocytes (Grande et al. (1989) J Orthop Res 7: 208; and Taligawa et al. (1987) Bone Miner 2: 449), and chondrocytes attached to natural or synthetic polymers (Walitani et al. (1989) J Bone Jt Surg 71B: 74; Vacanti et al. (1991) Plast Reconstr Surg 88: 753; von Schroeder et al. (1991) J Biomed Mater Res 25:329; Freed et al. (1993) J Biomed Mater Res 27: 11; and the Vacanti et al. U.S. Pat. No. 5,041,138). For example, chondrocytes can be grown in culture on biodegradable, biocompatible highly porous scaffolds formed from polymers such as polyglycolic acid, polylactic acid, agarose gel, or other polymers that degrade over time as a function of hydrolysis of the polymer backbone into innocuous monomers. The matrices are designed to allow adequate nutrient and gas exchange to the cells until engraftment occurs. The cells can be cultured in vitro until adequate cell volume and density has developed for the cells to be implanted. One advantage of the matrices is that they can be cast or molded into a desired shape on an individual basis, so that the final product closely resembles the patient's own ear or nose (by way of example), or flexible matrices can be used which allow for manipulation at the time of implantation, as in a joint.

These and other implants and prosthetics can be treated with and used to administer the subject antibodies or binding fragments thereof. For example, a composition including the antibody or binding fragment can be applied to or coated on the implant or prosthetic. In this way, the antibodies or fragments thereof can be administered directly to the specific affected tissue (e.g., to the damaged joint).

The subject antibodies can be administered as part of a combinatorial therapy with other agents. Combination therapy refers to any form of administration in combination of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can have a combined (conjoint) effect of different therapeutic compounds.

For example, in the case of inflammatory conditions, the subject antibodies can be administered in combination with one or more other agents useful in the treatment of inflammatory diseases or conditions. Agents useful in the treatment of inflammatory diseases or conditions include, but are not limited to, anti-inflammatory agents, or antiphlogistics. Antiphlogistics include, for example, glucocorticoids, such as cortisone, hydrocortisone, prednisone, prednisolone, fluorcortolone, triamcinolone, methylprednisolone, prednylidene, paramethasone, dexamethasone, betamethasone, beclomethasone, fluprednylidene, desoxymethasone, fluocinolone, flunethasone, diflucortolone, clocortolone, clobetasol and fluocortin butyl ester; immunosuppressive agents such as anti-TNF agents (e.g., etanercept, infliximab) and IL-1 inhibitors; penicillamine; non-steroidal anti-inflammatory drugs (NSAIDs) which encompass anti-inflammatory, analgesic, and antipyretic drugs such as salicyclic acid, celecoxib, difunisal and from substituted phenylacetic acid salts or 2-phenylpropionic acid salts, such as alclofenac, ibutenac, ibuprofen, clindanac, fenclorac, ketoprofen, fenoprofen, indoprofen, fenclofenac, diclofenac, flurbiprofen, piprofen, naproxen, benoxaprofen, carprofen and cicloprofen; oxican derivatives, such as piroxican; anthranilic acid derivatives, such as mefenamic acid, flufenamic acid, tolfenamic acid and meclofenamic acid, anilino-substituted nicotinic acid derivatives, such as the fenamates miflumic acid, clonixin and flunixin; heteroarylacetic acids wherein heteroaryl is a 2-indol-3-yl or pyrrol-2-yl group, such as indomethacin, oxmetacin, intrazol, acemetazin, cinmetacin, zomepirac, tolmetin, colpirac and tiaprofenic acid; idenylacetic acid of the sulindac type; analgesically active heteroaryloxyacetic acids, such as benzadac; phenylbutazone; etodolac; nabunetone; and disease modifying antirheumatic drugs (DMARDs) such as methotrexate, gold salts, hydroxychloroquine, sulfasalazine, ciclosporin, azathioprine, and leflunomide.

Other therapeutics useful in the treatment of inflammatory diseases or conditions include antioxidants. Antioxidants may be natural or synthetic. Antioxidants are, for example, superoxide dismutase (SOD), 21-aminosteroids/aminochromans, vitamin C or E, etc. Many other antioxidants are well known to those of skill in the art.

The subject antibodies may serve as part of a treatment regimen for an inflammatory condition, which may combine many different anti-inflammatory agents. For example, the subject antibodies may be administered in combination with one or more of an NSAID, DMARD, or immunosuppressant. In one embodiment of the application, the subject antibodies or fragments thereof may be administered in combination with methotrexate. In another embodiment, the subject antibodies may be administered in combination with a TNF-α inhibitor.

In the case of cardiovascular disease conditions, and particularly those arising from atherosclerotic plaques, which are thought to have a substantial inflammatory component, the subject antibodies can be administered in combination with one or more other agents useful in the treatment of cardiovascular diseases. Agents useful in the treatment of cardiovascular diseases include, but are not limited to, β-blockers such as carvedilol, metoprolol, bucindolol, bisoprolol, atenolol, propranolol, nadolol, timolol, pindolol, and labetalol; antiplatelet agents such as aspirin and ticlopidine; inhibitors of angiotensin-converting enzyme (ACE) such as captopril, enalapril, lisinopril, benazopril, fosinopril, quinapril, ramipril, spirapril, and moexipril; and lipid-lowering agents such as mevastatin, lovastatin, simvastatin, pravastatin, fluvastatin, atorvastatin, and rosuvastatin.

For the treatment of sepsis and sepsis-related disorders or conditions such as septic shock, anti-LOX-1 antibodies of the invention can be administered in combination with other agents and therapeutic regimens to treat sepsis and sepsis-related disorders or conditions. For example, sepsis can be treated by administering the subject antibodies in combination with antibiotics and/or other pharmaceutical compositions that are the standard of care for the particular symptoms and state of the patient.

In one embodiment, the present invention also provides a method for selectively inhibiting the interaction of one ligand with LOX-1, but not one other ligand for LOX-1, in a subject which comprises administering to the subject a therapeutically effective amount of a binding partner identified by the methods of the invention. A therapeutically effective amount is an amount that is capable of preventing interaction of a LOX-1 ligand with LOX-1 in a subject. Accordingly, the amount will vary with the subject being treated. Administration of the binding partner may be hourly, daily, weekly, monthly, yearly or a single event. For example, the effective amount of the compound may comprise from about 1 μg/kg body weight to about 100 mg/kg body weight. In one embodiment, the effective amount of the compound comprises from about 1 μg/kg body weight to about 50 mg/kg body weight. In one embodiment, the effective amount of the compound comprises from about 10 μg/kg body weight to about 10 mg/kg body weight. The actual effective amount will be established by dose/response assays using methods standard in the art (Johnson et al., Diabetes. 42:1179, (1993)). Thus, as is known to those in the art, the effective amount will depend on bioavailability, bioactivity, and biodegradability of the compound.

For example, the anti-LOX-1 antibodies and compositions of the invention are administered to a patient in need thereof in an amount sufficient to inhibit release of proinflammatory cytokine from a cell and/or to treat an inflammatory condition. The invention includes inhibiting release of a proinflammatory cytokine by at least 10%, 20%, 25%, 50%, 75%, 80%, 90%, or 95%, as assessed using methods described herein or other methods known in the art.

In one embodiment, the subject is an animal. In one embodiment, the subject is a human. In one embodiment, the subject is suffering from a LOX-1-related disease such as atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation.

The subject antibodies or binding fragments thereof can be administered in a dose of from about 1 μg/kg body weight to about 100 mg/kg body weight. In one embodiment, the effective amount of the compound comprises from about 1 μg/kg body weight to about 50 mg/kg body weight. The length frequency of treatment will depend upon inter alia the particular disease state as well as the state of the patient.

### EXAMPLES

The following examples demonstrate certain aspects of the present invention. However, it is to be understood that these examples are for illustration only and do not purport to be wholly definitive as to conditions and scope of this invention. It should be appreciated that when typical reaction conditions (e.g., temperature, reaction times, etc.) have been given, the conditions both above and below the specified ranges can also be used, though generally less conveniently. The examples are conducted at room temperature (about 23° C. to about 28° C.) and at atmospheric pressure. All parts and percents referred to herein are on a weight basis and all temperatures are expressed in degrees centigrade unless otherwise specified.

### Example 1

### Generation of Antibodies that Selectively Inhibit the Binding of One Ligand, But not One Other Ligand to Lox-1: Screen Number 1

A number of rat anti-mouse LOX-1 monoclonal antibodies were generated using standard methods known to those skilled in the art. The selectivity studies were done by analyzing the ability of the antibodies to selectively compete with CRP or oxLDL binding to a recombinant mouse LOX-1 Fc protein by an alphascreen binding assay. Several antibodies were identified which inhibited oxLDL binding to LOX-1, but not CRP binding to LOX-1. These were given the designations 11A2 (also referred to as LOX1-11A2) and 33F1 (also referred to as LOX1-33F1), shown in Tables 1 and 2. Furthermore, as shown in the table below, carrageenan, a non-selective LOX-1 agent was used as an internal control. Carrageenan inhibited CRP binding to LOX-1 with an IC50 equal to 0.149 ug/ml and 0.089 ug/ml for oxLDL with an oxLDL selectivity ratio of 1.678. Therefore, any antibody showing a 3-fold or greater selectivity for either ligand was sought. The most oxLDL selective antibodies included 11A2 and 33F1 with oxLDL selectivity ratios >3.5 compared to carrageenan. The oxLDL selectivity was determined by dividing the values in the bio-CRP+mFc-mLOX1 column by the values in the Bio-oxLDL+mFc-mLOX1 column, shown below. Any antibody having a value greater than 3.0 was considered to be selective for oxLDL.

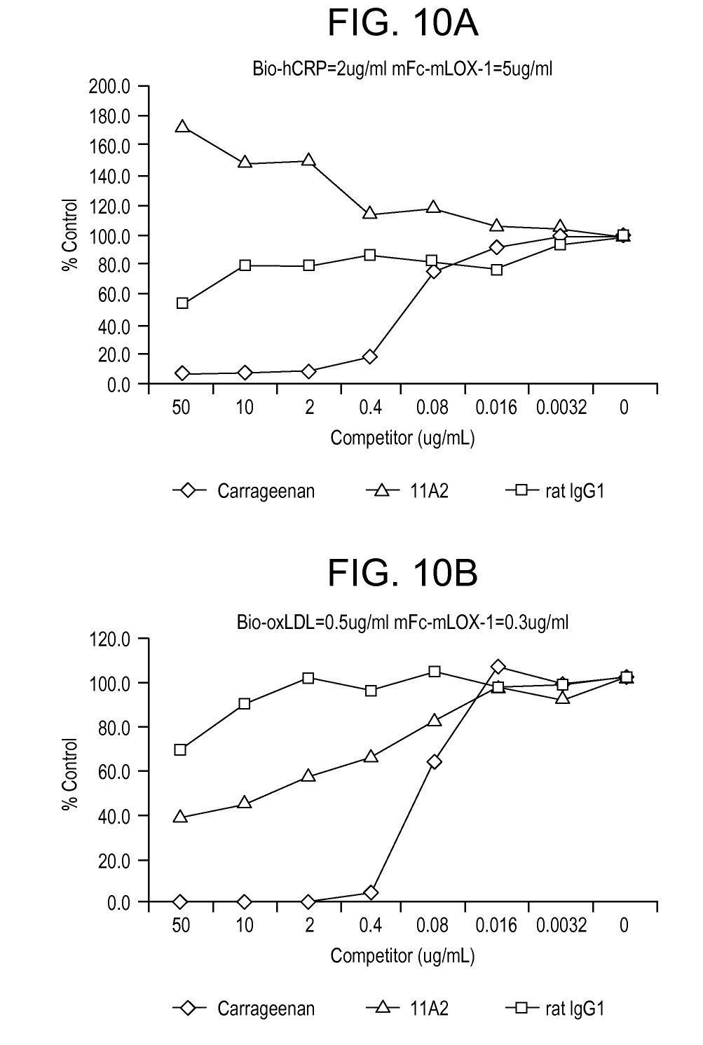

FIG. 10 shows the binding curves that demonstrates the ability of antibody 11A2 to selectively compete oxidized low density lipoprotein (oxLDL), but not C reactive protein (CRP), binding to a recombinant mouse LOX-1 Fc protein, as shown using an ALPHAscreen binding assay. Carrageenan, a non-selective LOX-1 agent, was able to non-selectively compete for both oxLDL and CRP binding to mouse LOX-1 Fc protein, while rat IgG1 did not compete for either oxLDL and CRP binding to mouse LOX-1 Fc protein.

The variable light chain and the variable heavy chain nucleic acid and protein sequences of the 11A2 and 33F1 antibodies were sequenced and the sequences shown in FIGS. 8 and 9. The particular sequence identifiers, with and without the signal sequences, are shown in Table 3.

The variable light chain (VL) of the 11A2 antibody, shown in FIG. 8, has a signal sequence encoded by nucleotides at position 1 through 60. The CDRs of 11A2 VL are encoded by nucleotides at the following positions: 130-162; 208-228 and 325-351.

The variable heavy chain (VH) of the 11A2 antibody, shown in FIG. 8, has a signal sequence encoded by nucleotides at position 1 through 57. The CDRs of 11A2 VH are encoded by nucleotides at the following positions:144-162; 205-255 and 351-387.

The variable light chain (VL) of the 33F1 antibody, shown in FIG. 9, has a signal sequence encoded by nucleotides at position 1 through 60. The CDRs of 33F1 VL are encoded by nucleotides at the following positions:130-180; 226-246 and 343-366.

The variable heavy chain (VH) of the 33F1 antibody, shown in FIG. 9, has a signal sequence encoded by nucleotides at position 1 through 57. The CDRs of 33F1 VH are encoded by nucleotides at the following positions:145-162; 205-252 and 349-384.

TABLE 1

| | Bio-CRP + | Bio-oxLDL + | | |
|---|---|---|---|---|
| | mFc-mLOX1 | mFc-mLOX1 | OxLDL | Adjusted |
| Competitor | IC50 (ug/ml) | IC50 (ug/ml) | selectivity | oxLDL sel |
| 11A2 | n/a | 0.127 | | > |
| 33F1 | 0.193 | 0.030 | 6.507 | 3.9 |
| 15F11 | 0.657 | 0.334 | 1.965 | 1.2 |
| Carrageenan | 0.149 | 0.089 | 1.678 | 1.0 |

### Example 2

### Generation of Antibodies that Selectively Inhibit the Binding of One Ligand, But not One Other Ligand to Lox-1: Screen Number 2

Further studies were done to test the ability of additional rat anti-mouse LOX-1 antibodies to selectively compete oxLDL vs CRP binding to a recombinant mouse LOX-1 Fc protein by analysis in an alphascreen binding assay. As shown in the table below, carrageenan, a non-selective LOX-1 agent was used as an internal control. Carrageenan inhibited CRP binding to LOX-1 with an IC50 equal to 0.149 ug/ml and 0.089 ug/ml for oxLDL with an oxLDL selectivity ratio of 1.678. Therefore, any antibody showing a 3-fold or greater selectivity for either ligand was sought. Several antibodies were identified which inhibited oxLDL binding to LOX-1, but not CRP binding to LOX-1. The most oxLDL selective antibodies included 5E1, 33F1 and 11A2 with oxLDL selectivity ratios >3.0 compared to carrageenan.

TABLE 2

| | Bio-CRP + | Bio-oxLDL + | | |
|---|---|---|---|---|
| | mFc-mLOX1 | mFc-mLOX1 | oxLDL | Adjusted |
| Competitor | IC50 (ug/ml) | IC50 (ug/ml) | selectivity | oxLDL sel |
| 5E1 | 0.204 | 0.038 | 5.405 | 3.2 |
| 11A2 | n/a | 0.127 | | > |
| 21G2 | 0.068 | 0.016 | 4.146 | 2.5 |
| 33F1 | 0.193 | 0.030 | 6.507 | 3.9 |
| 5F1 | 0.213 | 0.061 | 3.477 | 2.1 |
| 1H7 | 0.346 | 0.073 | 4.756 | 2.8 |
| 15F12 | 0.500 | 0.104 | 4.818 | 2.9 |
| 4F2 | n/a | 6.093 | | > |
| 12G5 | 0.157 | 0.055 | 2.847 | 1.7 |
| 1C9 | n/a | n/a | | |
| 8F3 | 0.886 | 0.264 | 3.351 | 2.0 |
| 11G9 | n/a | n/a | | |
| 31G4 | 0.047 | 0.031 | 1.480 | 0.9 |
| 13E9 | 0.000 | 10.000 | | > |
| 29C3 | 0.000 | 40.000 | | > |
| 15F11 | 0.657 | 0.334 | 1.965 | 1.2 |
| Carrageenan | 0.149 | 0.089 | 1.678 | 1.0 |

### Example 3

### The Identification of C Reactive Protein as a Novel Ligand for the Oxidized LDL Receptor, LOX-1

### Materials and Methods

### Reagents and Antibodies

Endotoxin-free, azide-free, purified recombinant human CRP (endotoxin level <0.1 EU per 1 μg protein, R&D Systems), purified recombinant human LOX1 comprised of the extracellular domain (ECD) of human LOX-1 (amino acids 61-273) with an N-terminal polyhistidine tag (His-LOX-1; R&D Systems), His-neuturin control protein from R&D Systems, goat anti-CRP polyclonal antibody, goat anti-LOX1 polyclonal antibody were purchased from R&D Systems. A rabbit anti-CRP polyclonal antibody was purchased from Calbiochem. A monoclonal anti-LOX-1 antibody was purchased from CellSciences. Human oxidized LDL was from Intracel. The siRNAs (human LOX-1 (OLR1) ON-TARGET plus smart pool or siRNA CONTROL non-targeting siRNA) were purchased from Dharmacon (Lafayette, Colo.).

### Generation of LOX1 Arginine to Glutamine Mmutants

Single point mutants (R208N, R229N, R231N, R248N) for LOX-1 were generated using QuikChange site-directed mutagenesis kit (Stratagene) with wildtype LOX-1 pAdori expression vector as PCR template. The primers used for the PCR reactions are:

R208N;

(SEQ ID NO: 17)

Forward primer:
5′-CATTCTGGATGGGGCTGTCTAATAGGAACCCC
AGCTACCCATG3′;

(SEQ ID NO: 18)

Reverse primer:
5′-CATGGGTAGCTGGGGTTCCTATTAG ACAGCCCCATCCAGAATG-
3′.
R229N;

(SEQ ID NO: 19)

Forward primer:
5′-CCTTTGATGCCCC ACT
TATTTAATGTCCGAGGCGCTGTCTCC-3′;

(SEQ ID NO: 20)

Reverse primer:
5′-GGAGACAG CGCCTCGGACATTAAATAAGTGGGGCATCAAAGG-3′.
R231N:

(SEQ ID NO: 21)

Forward primer:
5′-GATGCCCCACTTATTTAGAGTCAATGGCGCTGTCTCCCAGACATAC-
3′

(SEQ ID NO: 22)

Reverse primer:
5′-GTATGTCTGGGAGACAGCGCCATTGACTCTAAATAA
GTGGGGCATC-3′.
R248N:

(SEQ ID NO: 23)

Forward primer:
5′-CAGGTACCTGTGCATATATACA AAATGGAGCTGTTTATGCGGAAAA
C-3′;

(SEQ ID NO: 24)

Reverse primer:
5′-GTTTTCCGCATAAACAGCTCCATTTTGTATATATGCACAGGTACCT
G-3′

Composite mutant LOX-1 with all four arginine resides mutated to asparagines was generated by consecutive rounds of Pfu PCR-based mutagenesis, each round using LOX-1 mutant generated from the previous round of mutagenesis. All constructs were sequence confirmed to contain the correct mutations.

LOX-1 Ligand Binding ELISA

CRP or oxLDL was coated on ELISA plate overnight at 4° C. After washing and blocking with assay buffer (PBS with 0.1% Tween 20 and 1% BSA), the plate was incubated with His-LOX-1 for one hour at room temperature. The plate was washed, and bound His-LOX-1 was allowed to interact with goat-anti-LOX-1 polyclonal antibody for one hour at room temperature. After washing, an HRP conjugated anti-goat secondary antibody was added and the plate was incubated for one hour at room temperature. The binding was detected using TMB substrate system and OD450 was determined on a plate reader.

### Alpha Screen Assay

The interaction of LOX-1 with CRP proteins was confirmed with Alphascreen assay, PerkinElmer AlphaScreen® Histidine (Nickel Chelate) detection kit, 6760619M. For binding assay, His-LOX-1 (1 ug/ml) was bound to nickel chelate acceptor beads and incubated with increasing concentrations of biotin-CRP or biotin-oxLDL bound to streptavidin-coated donor beads. For the competition assay, His-LOX-1 (1 ug/ml) and biotin-CRP (1 ug/ml) or His-LOX-1 (0.5 ug/ml) and biotin-oxLDL (0.3 ug/ml) were incubated with increasing concentrations of competitors along with nickel chelate acceptor beads and streptavidin-coated donor beads. The incubation was carried out in a 384-well plate for 2 hours before measuring the interaction signal in FusionAlpha plate reader (Perkin Elmer). Experiments were performed at least three times and a representative experiment is shown.

### FACS

DI-oxLDL (0-100 μg/ml) or CRP (0-200 μg/ml) diluted in 2% FCS/PBS was incubated with 10⁶ CHO/LOX-1 cells or CHO/Mock cells for 60 mins at 4° C. DI-oxLDL could be directly detected by FACS. CRP binding to the cell surface LOX-1 was detected by incubation with 5 μg/ml rabbit anti-CRP antibody followed by 5 μg/ml Alex596 conjugated anti-rabbit secondary antibody for 30 mins at room temperature. Cells were analyzed using a Becton Dickinson FACSCalibur® flow cytometer with CellQuest software (Becton Dickinson). Results are presented as the geometric mean fluorescence intensity (MFI).

### Binding of CRP to Cell Surface LOX-1

A cDNA clone containing human LOX-1 open reading frame was obtained from Origene. A CHO cell line stably expressing human LOX-1 was established and maintained in alpha medium containing 10% heat-inactivated and dialyzed FBS and 25 nM methotrexate (Sigma). To detect CRP binding to cell surface LOX-1, CHO-LOX-1 cells were incubated with CRP added to CHO-LOX-1 growth medium for 1 h at 4° C. The cells were fixed with 2% paraformaldhyde/PBS for 20 min at 4° C., washed, and incubated with rabbit anti-CRP antibody and goat anti-LOX-1 antibody for 1 h at room temperature. After washing, the cells were incubated with Alex596 conjugated anti-rabbit and Alexa488 conjugated anti-goat secondary antibodies for 1 h at room temperature. The cells were washed and counterstained with Hoechst dye and photographed under fluorescent microscope at 40× magnification. To detect the inhibition of anti-LOX-1 antibody to the binding of LOX-1 to CRP, the cells were pre-incubated with control or anti-LOX-1 monoclonal antibody for 1 h at 4° C. The cells were washed and CRP was added to the cells. The bound CRP was detected as described above.

### HAECT-1 Cell Culture

Transformed human aortic endothelial cells (HAECT-1) (Chadwick C C, Shaw L J, Winneker R C. Experimental Cell Research. 1998; 239:423-429) were cultured in EGM-2 (Lonza) containing 2% FBS, 0.04% hydrocortisone, 0.4% hFGF, 0.1% VEGF, 0.1% R3-IgF, 0.1% ascorbic acid, 0.1% hEGF, 0.1% GA-1000 and 0.1% heparin in Falcon T-175 culture flasks at 34° C. in 5% CO₂. The cells were plated in a 24-well dish at 150,000 cells/well overnight. The cells were pretreated for 1 hr with antibody prior to CRP treatment for 24 hrs. The RNA was purified using Qiagen RNeasy following the manufacturer's protocol and gene expression determined by real time RT-PCR using an ABI 7900.

### SiRNA Transfection

Human aortic endothelial (HAECT) cells at 85-90% confluence were cultured as described above, and were plated in a 96 well dish at 75,000 cells/well and were transfected overnight with 100 nM final concentration of human LOX-1 (OLR1) ON-TARGETplus smart pool small interfering RNA (siRNA), Toll-Like Receptor-4 (TLR4) siRNA or siCONTROL non-targeting siRNA using DharmaFECT-1 (Dharmacon, Lafayette, Colo.) according to the manufacturer's protocol.

The siRNAs specific for LOX-1 include the following sequences:

| | | |
|---|---|---|
| Sequence 1: | GGAUUAGUAGUGACCAUUA; | (SEQ ID NO: 25) |
| Sequence 2: | UGUCUGACCUCCUAACACA; | (SEQ ID NO: 26) |
| Sequence 3: | CAGAAGAAGGCAAACCUAA; | (SEQ ID NO: 27) |
| Sequence 4: | GAGAAGUGCUUGUCUUUGG. | (SEQ ID NO: 28) |

Cells were treated with 10 ug recombinant human CRP (R&D Systems) for 24 hrs post transfection. Total RNA was isolated using the Applied Biosystems RNA purification kit per manufacturer's protocol. The mRNA gene expression levels of LOX-1, Interleukin-8 (IL-8), ICAM-1 and VCAM-1 were quantified by real time RT-PCR using the Qiagen Quantitech kit using an ABI 7900. The relative amount of mRNA was normalized to 18S. *p<0.05 versus control siRNA transfected cells.

### Results:

### Direct Interaction of Purified Recombinant LOX-1 and CRP

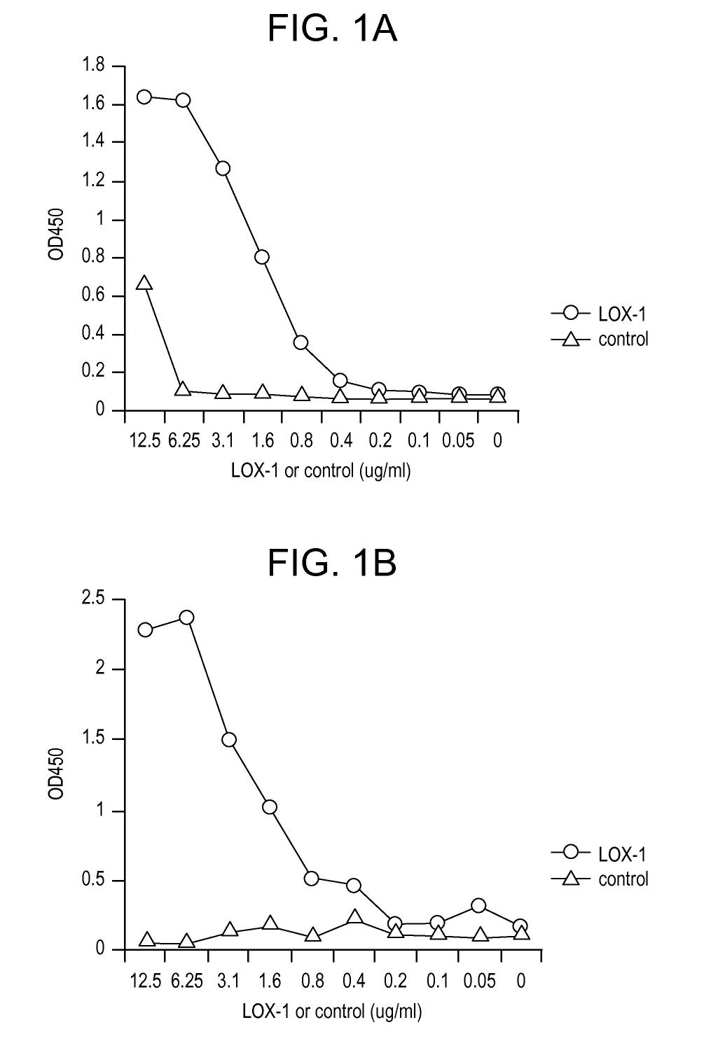

To examine whether CRP can function as a ligand for LOX-1, it was determined whether these two proteins interact directly in a purified system using two different assay formats. In each assay format, the binding of purified recombinant human CRP was compared with the well-characterized LOX-1 ligand, oxLDL to a human LOX-1 extracellular domain his-tagged protein (HIS-LOX). In the ELISA assay, oxLDL (FIG. 1A) or CRP (FIG. 1B) was coated on the well of the microtiter plate overnight at 4° C. After washing and blocking with assay buffer, the plate was incubated with His-LOX-1 for one hour at room temperature. The plate was washed, and bound His-LOX-1 was allowed to interact with goat-anti-LOX-1 polyclonal antibody for one hour at room temperature. The binding was detected using TMB substrate system and OD450 was determined on a plate reader, and binding of His-LOX-1 was detected with a streptavidin antibody. In this format, a dose-dependent interaction of His-LOX-1 with both oxLDL and CRP was observed. (FIGS. 1A and 1B) A His-tagged control protein (His-Neutrurin) exhibited only background signals, indicating that the binding of His-LOX-1 to both oxLDL and CRP was specific.

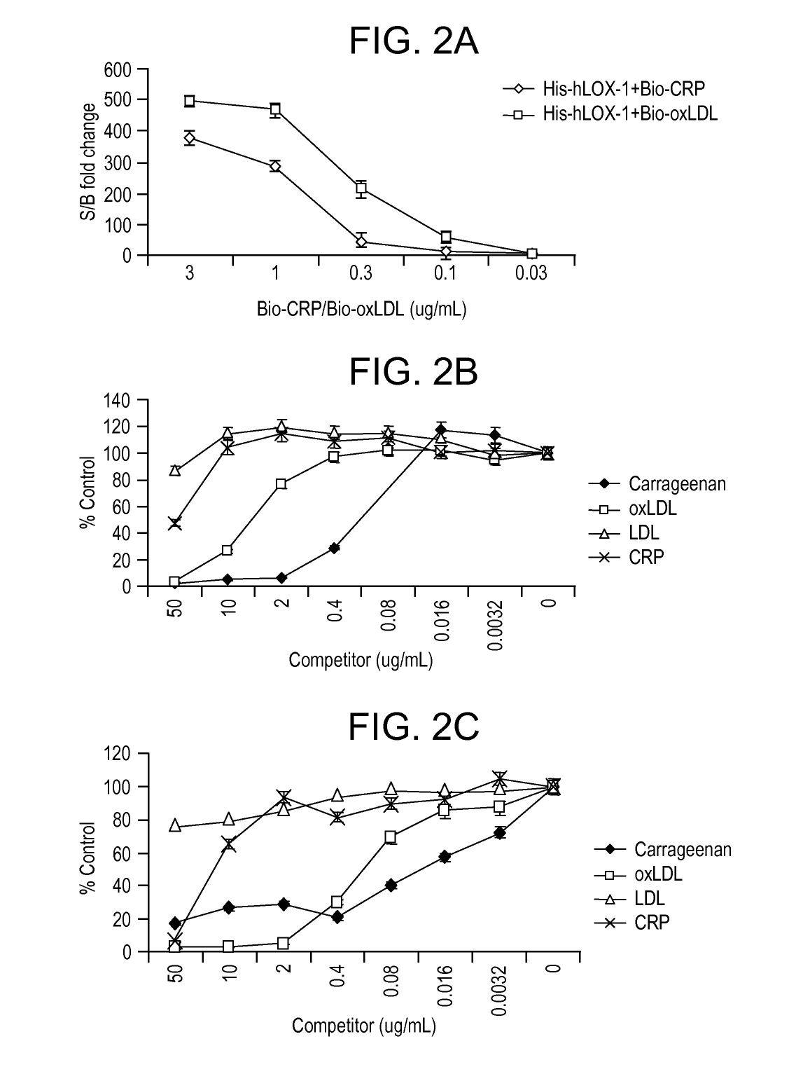

An electrochemical proximity based alphascreen assay was also performed to confirm the CRP interaction with LOX-1. In this assay, (A) His-LOX-1 (1 ug/ml) was bound to nickel chelate acceptor beads and incubated with increasing concentrations of biotin-CRP or biotin-oxLDL bound to streptavidin-coated donor beads. For the competition assay, (B) His-LOX-1 (0.5 ug/ml) and biotin-oxLDL (0.3 ug/ml) or (C) His-LOX-1 (1 ug/ml) and biotin-CRP (1 ug/ml) were incubated with increasing concentrations of competitors as indicated along with nickel chelate acceptor beads and streptavidin-coated donor beads. The incubation was incubated for 2 hours before measuring the interaction signal in FusionAlpha plate reader. Experiments were performed at least three times and a representative experiment is shown. As shown in FIG. 2A, oxLDL and CRP dose-dependently bound to LOX-1 consistent with the ELISA data. To address the specificity of binding of CRP to LOX-1, competition experiments were performed. The LOX-1 antagonist, carrageenan was an effective inhibitor of both CRP and oxLDL interaction with LOX-1 (FIGS. 2B & 2C). While oxLDL could also compete for CRP binding to LOX-1 (FIG. 2B), CRP was not an effective inhibitor of oxLDL-LOX-1 interaction (FIG. 2C). As expected, LDL, a molecule unable to bind LOX-1, failed to compete off the binding of either CRP or oxLDL. These data suggest that CRP binding to LOX-1 may occur through distinct sites relative to oxLDL while carrageenan bridges both oxLDL and CRP interaction sites. Taken together, these data demonstrate that CRP can interact with the extracellular domain of LOX-1.

### Interaction of CRP to LOX-1 on Cell Surface

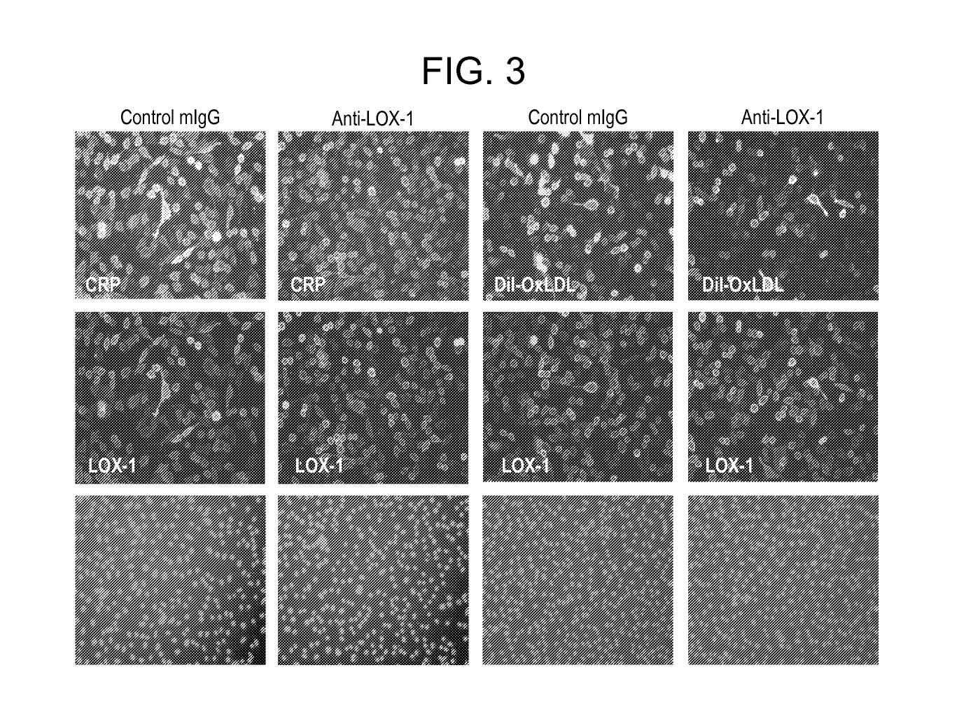

Whether CRP can bind to cell surface expressed LOX-1 was examined. CHO cells stably expressing human LOX-1 were generated (CHO/LOX-1) and used in the binding study. Purified recombinant human CRP was allowed to interact with human LOX-1 on the surface of CHO cells and the binding was detected by immunofluorescent staining using an anti-CRP antibody and Alexa488-conjugated secondary antibodies. More particularly, CRP and Dil-oxLDL were added to CHO-LOX-1 growth medium for 1 h at 4° C. The cells were incubated with rabbit anti-CRP antibody and goat anti-LOX-1 antibody for 1 h at room temperature and then incubated with Alex596 conjugated anti-rabbit and Alexa488 conjugated anti-goat secondary antibodies for 1 h. To detect the inhibition of anti-LOX-1 antibody to the binding of LOX-1 to CRP, the cells were pre-incubated with control or anti-LOX-1 monoclonal antibody for 1 h at 4° C. As shown in FIG. 3, CRP and Dil-oxLDL binding on CHO-LOX-1 cells was detected while no binding was observed on the WT control CHO cells (data not shown). The binding of CRP to the CHO/LOX-1 cells was specific for LOX-1 since its binding, as well as Dil-oxLDL, was attenuated using an anti-LOX-1 neutralizing antibody but not in the presence of an anti-IgG control antibody.

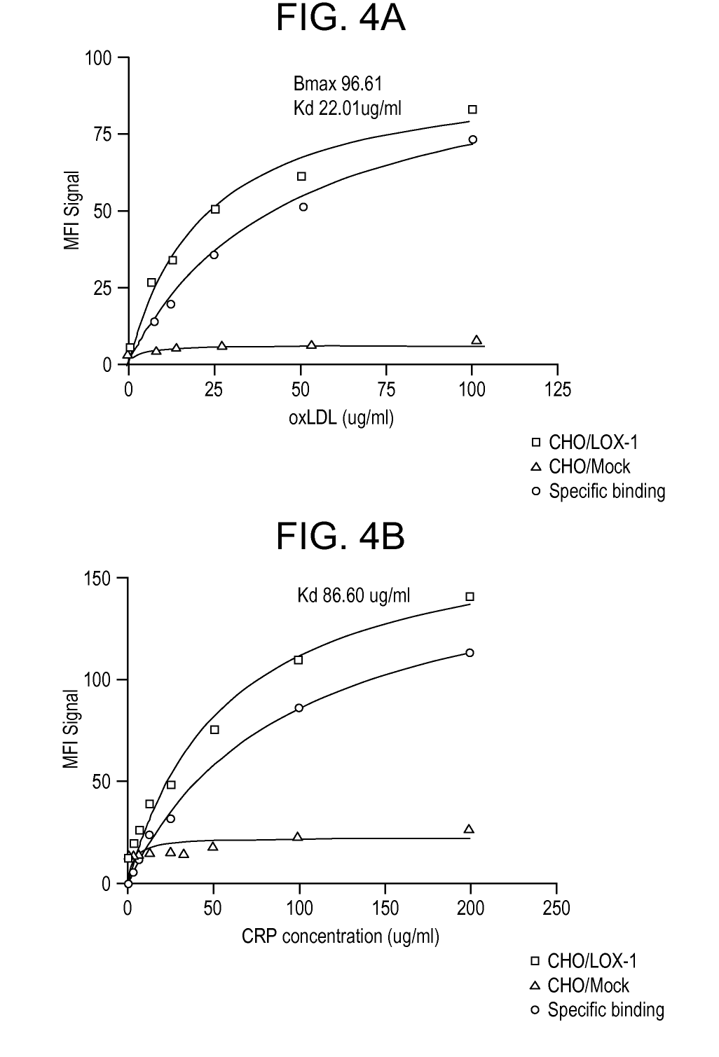

To extend these results, FACS analysis was conducted to determine the binding affinity of CRP for membrane expressed LOX-1 on the CHO/LOX-1 cells. In FIG. 4A, CHO/LOX-1 or CHO/Mock cells were incubated with increasing doses of DI-oxLDL (0-100 ug/ml) for 60 mins at 4° C. DI-oxLDL could be directly detected by FACS. In FIG. 4B, (B) CHO/LOX-1 or CHO/Mock cells were incubated with increasing doses of CRP (0-200 ug/ml) for 60 mins at 4° C. CRP binding to the cell surface LOX-1 was detected by incubation with 5 μg/ml rabbit anti-CRP antibody followed by 5 μg/ml Alex596 conjugated anti-rabbit secondary antibody for 30 mins at room temperature. As a positive control comparator, the binding affinity of oxLDL to LOX-1 was determined and calculated to be 22 ug/ml (FIG. 4A); comparable to previous results using CHO cells over-expressing bovine LOX-1 (Moriwaki H, Kume N, Sawamura T, Aoyama T, Hoshikawa H, Ochi H, Nishi E, Masaki T, Kita T. Arteriosclerosis, Thrombosis & Vascular Biology. 1998; 18:1541-1547). CRP binding to the CHO/LOX-1 cells was also dose dependent with a binding affinity of 86 ug/ml (FIG. 4B) with only background binding observed in the mock control CHO cells.

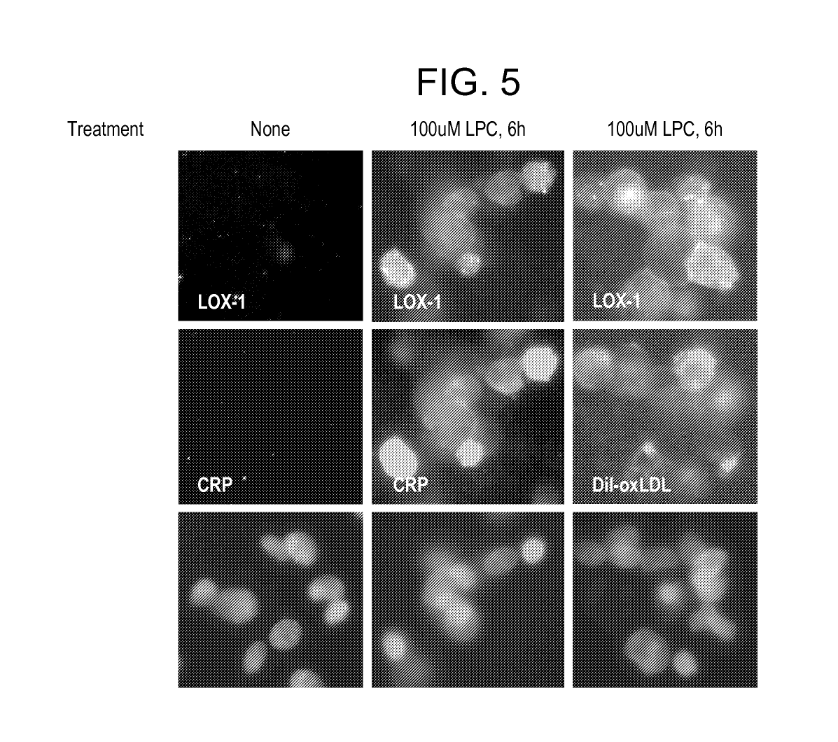

This interaction of CRP with cell-surface LOX-1 was confirmed in human aortic endothelial cells expressing endogenous LOX-1. In particular, HAECT-1 cells were incubated with 100 uM LPC for 6 hours. CRP and Dil-oxLDL were added to the growth medium for 1 h at 4° C. The cells were incubated with rabbit anti-CRP antibody and goat anti-LOX-1 antibody for 1 hour at room temperature and then incubated with Alex596 conjugated anti-rabbit and Alexa488 conjugated anti-goat secondary antibodies for 1 hour. DI-oxLDL binding could be directly detected. To detect the inhibition of anti-LOX-1 antibody to the binding of LOX-1 to CRP, the cells were pre-incubated with control or anti-LOX-1 monoclonal antibody for 1 h at 4° C. In these experiments, the expression of endogenous LOX-1 on the cell surface was non-detectable in untreated cells but largely increased after the cells were stimulated with a 6 hour treatment with 100 μM LPC (FIG. 5). Specific binding of CRP was observed only on the HAECT-1 cells stimulated with LPC with very low level of binding observed on the unstimulated cells, consistent with the relative expression pattern of LOX-1. As a positive control, the binding of Dil-oxLDL to LOX-1 was also only observed on the stimulated HAECT-1 cells. Together, these results confirm the in vitro binding data and indicate that CRP can interact with cell-surface LOX-1.

The Mechanism of LOX-1 Binding to CRP is Distinct from oxLDL Binding

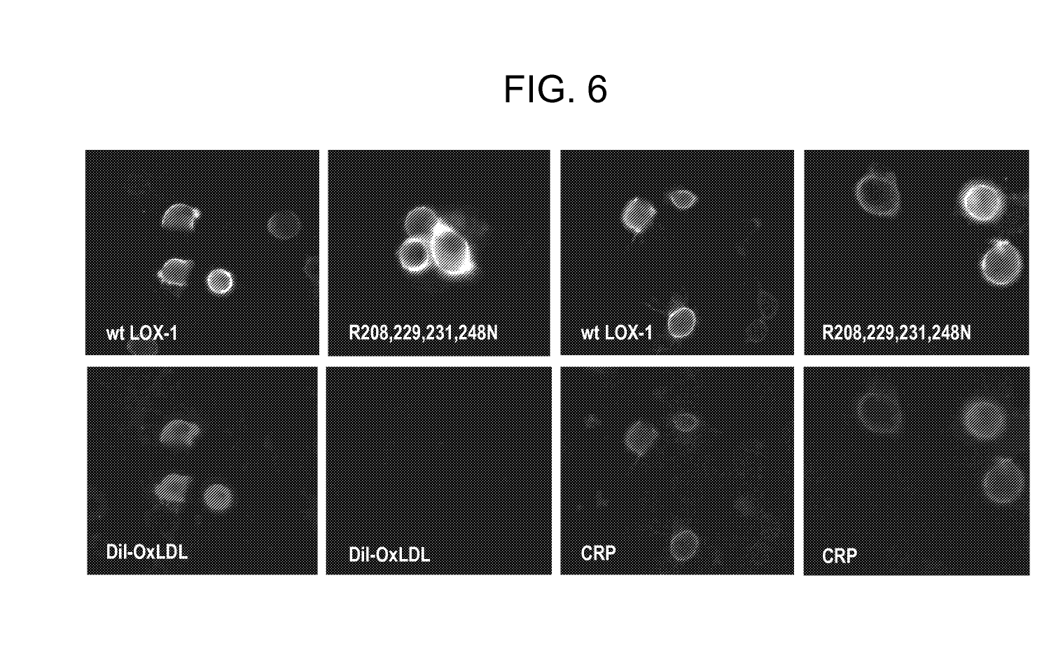

The crystal structure of LOX-1 suggests that arginine 208, 229, 231, and 248 form a “basic spine” on the ligand binding interface and mediate a charge-to-charge interaction with negatively-charged ligand (Ohki I, Ishigaki T, Oyama T, Matsunaga S, Xie Q, Ohnishi-S Kameyama M, Murata T, Tsuchiya D, Machida S, Morikawa K, Tate S. Structure. 2005; 13:905-917; Park H, Adsit F G, Boyington J C. J Biol Chem. 2005; 280:13593-13599). Mutagenesis studies have confirmed that mutations of each of these arginine residues led to partial to complete loss of binding between LOX-1 and acetylated LDL (Ohki I, Ishigaki T, Oyama T, Matsunaga S, Xie Q, Ohnishi-Kameyama M, Murata T, Tsuchiya D, Machida S, Morikawa K, Tate S. Structure. 2005; 13:905-917). To investigate the binding mechanisms by which LOX-1 interacts with CRP, LOX-1 mutants were generated to mutate each arginine 208, 229, 231, and 248 in LOX-1 to glutamine as performed by Ohki et al (Ohki I, Ishigaki T, Oyama T, Matsunaga S, Xie Q, Ohnishi-Kameyama M, Murata T, Tsuchiya D, Machida S, Morikawa K, Tate S. Structure. 2005; 13:905-917). In addition, a combinatorial mutant was generated changing all four arginine residues to glutamine. CHO cells transiently expressing LOX-1 containing all four arginine mutants were used to assess the binding specificity of recombinant CRP to LOX-1. The binding of recombinant CRP as well as Ox-LDL to wild type LOX-1 and an arginine mutant LOX-1 transiently expressed on the surface of transfected CHO cells was determined as described above for FIG. 3. Mutating each of these arginine residues individually in LOX-1 attenuated the binding of oxLDL (data not shown) while mutating all four arginine residues completely abolished oxLDL binding (FIG. 6). Mutating each or all of these arginines did not affect CRP binding (FIG. 6 and data not shown for individual arginine mutants). These results indicate that although the arginine residues are necessary for LOX-1 interaction with oxLDL, they are dispensable for CRP interaction and consistent with the in vitro competitive binding data indicating that CRP interaction with LOX-1 may be distinct from oxLDL.

### CRP Activates the Expression of Pro-Inflammatory Genes Through LOX1 in Endothelial Cells

CRP was previously reported to elicit inflammatory responses in endothelial cells through induction of LOX-1 expression resulting in the increase of monocyte adhesion and oxLDL uptake (Li L, Roumeliotis N, Sawamura T, Renier G. Circ Res. 2004; 95:877-883). To further link whether the biological effects of CRP were dependent upon LOX-1 signaling, studies were done to determine whether CRP could induce transcriptional responses through LOX-1. To address this, two types of studies were performed.

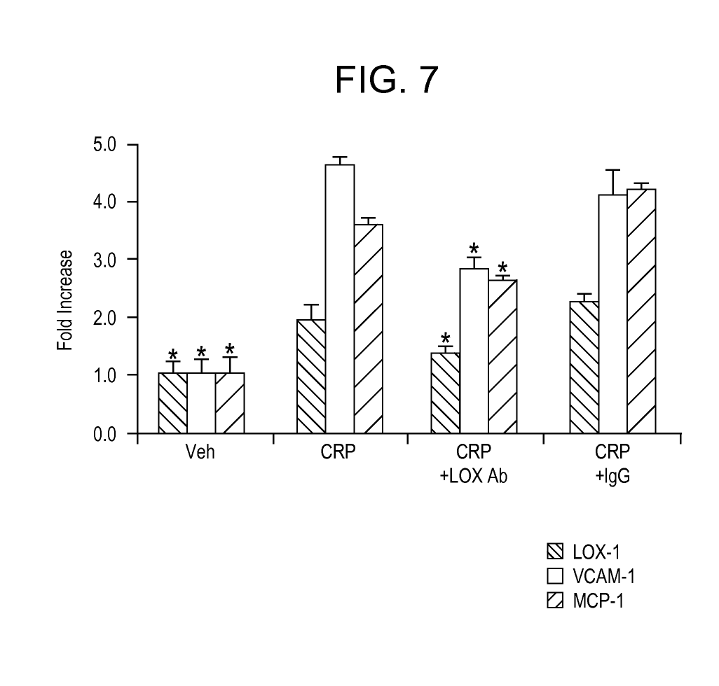

In one study, HAECT-1 cells were pretreated for 1 hour with the anti-LOX-1 or control IgG antibody prior to 10 ug/ml CRP treatment for 24 hrs. The gene expression analysis was determined by real time RT-PCR. As shown in FIG. 7, treatment of HAECT-1 cells with recombinant CRP induced the expression of LOX-1, MCP-1, and VCAM-1. These inductions were inhibited in the presence of an anti-LOX-1 antibody but not with an anti-IgG control antibody.

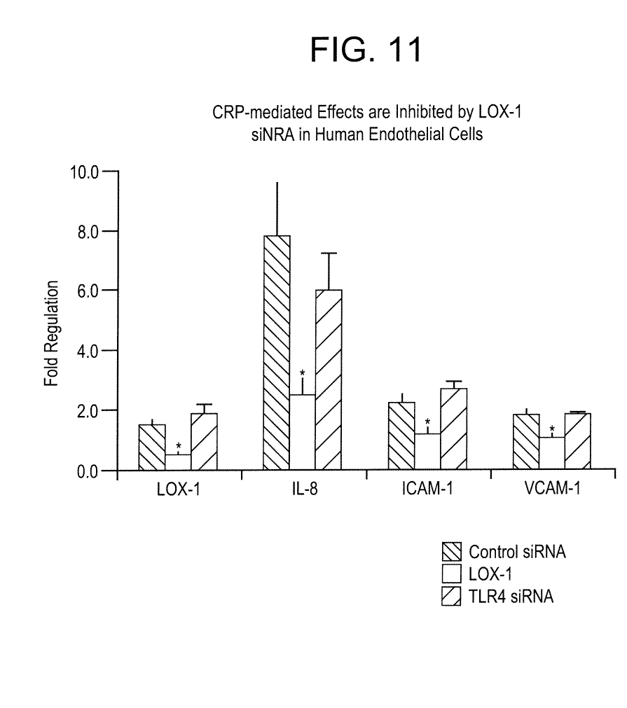

In a second study, a recombinant CRP preparation that is both azide-free and endotoxin-free was tested to determine if CRP could induce biologic responses through LOX-1. In this study, HAECT-1 cells were transfected for 24 hours with LOX-1, Toll-Like Receptor-4 (TLR4) or control siRNA prior to 10 μg/ml CRP treatment for 24 hours. LOX-1, Interleukin-8 (IL-8), ICAM-1 and VCAM-1 mRNA levels were determined by real time RT-PCR normalized to 18S endogenous control and expressed as fold regulation over vehicle treated controls. *p<0.05 versus control siRNA transfected cells. As shown in FIG. 11, treatment of HAECT-1 cells with LOX-1 siRNA resulted in a 50% reduction in LOX-1 mRNA levels compared to the control siRNA treated cells. This reduction of LOX-1 resulted in a lack of CRP induced expression of IL-8, ICAM-1 and VCAM-1 gene expression compared to the control siRNA treated cells. As a control, CRP treatment and induction of IL-8, ICAM-1 and VCAM-1 gene expression was not sensitive to interference by TLR4 siRNA suggesting that the effects of CRP are not due to endotoxin contamination.

Therefore, these results establish that CRP can mediate its effects through LOX-1 activation.

### Discussion

CRP is a strong predictor for cardiovascular events (Verma S, Szmitko P E, Ridker P M. Nature Clinical Practice Cardiovascular Medicine. 2005; 2:29-36; quiz 58; de Ferranti S D, Rifai N. Cardiovasc Pathol. 2007; 16:14-21). Among its biological effects, CRP has been reported to induce endothelial dysfunction through activation of complement, repression of nitric oxide release, upregulation of adhesion molecule expression and monocyte adherence. The activation of the scavenger receptor LOX-1 has been shown to result in the activation of similar signaling pathways involved in endothelial dysfunction. LOX-1 mediates the uptake of oxLDL also leading to the repression of nitric oxide signaling, induction of adhesion molecule expression, monocyte adherence and induction of pro-inflammatory pathways (Mehta J L, Chen J, Hermonat P L, Romeo F, Novelli G. Cardiovasc Res. 2006; 69:36-45).

CRP is demonstrated to be a novel ligand for LOX-1 and the interaction of CRP with LOX-1 results in the induction of endothelial inflammation. Human recombinant CRP was demonstrated to interact with the extracellular domain of human LOX-1 by ELISA and alphascreen proximity assays. Based on both the crystal structure for LOX-1 and site-directed mutagenesis studies, Ohki et al. have specifically identified four arginine residues (Arg208. Arg229, Arg231, Arg248) that constitutes the basic spine (Ohki I, Ishigaki T, Oyama T, Matsunaga S, Xie Q, Ohnishi-Kameyama M, Murata T, Tsuchiya D, Machida S, Morikawa K, Tate S. Structure. 2005; 13:905-917) that are critical for oxLDL binding to LOX-1. These arginine residues mediate charge-to-charge interactions, which presumably allows LOX-1 to interact with diverse, structurally unrelated ligands. To investigate whether this arginine spine was also critical for CRP-LOX-1 interaction, a LOX-1 construct in which the four arginine residues were substituted with glutamine was expressed on CHO cells. While this arginine spine was confirmed to be critical for oxLDL binding to LOX-1, it was dispensable for CRP binding. This is consistent with the inability of CRP to act as a competitor of oxLDL binding to LOX-1. Although oxLDL is an efficient competitor for CRP binding to LOX-1, this may not simply be due to competition for common binding sites. The binding affinity of oxLDL for LOX-1 is higher than CRP's and coupled with the large size of oxLDL, may simply sterically hinder CRP's access to the binding pocket. However, CRP likely shares some common binding components with oxLDL since another well-characterized LOX-1 ligand, carrageenan, and an anti-LOX-1 antibody were able to act as competitors to both.

A number of studies have proposed a direct impact of CRP on eliciting endothelial inflammation (Verma S, Szmitko P E, Ridker P M. Nature Clinical Practice Cardiovascular Medicine. 2005; 2:29-36; quiz 58; Venugopal S K, Devaraj S, Jialal I. Current Opinion in Nephrology & Hypertension. 2005; 14:33-37). These results suggest that some of these effects may be mediated through LOX-1. Minimal binding of CRP was observed in unstimulated HAECT-1 cells but was dramatically induced after LPC treatment, coincident with LOX-1 induction. In addition, treatment of human endothelial cells with CRP resulted in the induction of LOX-1 and its downstream genes, VCAM-1 and MCP-1, which was attenuated with an anti-LOX-1 neutralizing antibody. CRP interacts through the Fcγ receptors, FcγRI, FcγRIIa and/or FcγRIIb expressed on aortic endothelial cells to mediate its biologic effects (Devaraj S, Du Clos T W, Jialal I. Arterioscler Thromb Vasc Biol 2005; 25:1359-1363; Mineo C, Gormley A K, Yuhanna I S, Osborne-Lawrence S, Gibson L L, Hahner L, Shohet R V, Black S, Salmon J E, Samols D, Karp D R, Thomas G D, Shaul P W. Circulation Research. 2005; 97: 1124-1131). However, expression of these Fcγ receptors was not detected by real time RT-PCR in immortalized HAECT-1 cells in either unstimulated or stimulated cells suggesting that the effects of CRP observed are indeed mediated through LOX-1, thus explaining why no CRP binding was observed with the unstimulated cells.

The binding affinity for CRP interaction with LOX-1 was calculated to be 86 ug/ml (747 nM) with the LOX/CHO cells compared to 88 nM calculated for the Fc gamma receptors in primary human aortic endothelial cells (Devaraj S, Du Clos T W, Jialal I. Arterioscler Thromb Vasc Biol. 2005; 25:1359-1363). Despite the weaker binding affinity for LOX-1, it was demonstrated that physiologically relevant levels of CRP are capable of inducing downstream inflammatory responses in HAECT-1 cells via LOX-1. The LOX-1 sensitive regulation of VCAM-1 observed here has also been previously reported to be associated with CRP functioning via the Fcγ receptors (Devaraj S, Du Clos T W, Jialal I. Arterioscler Thromb Vasc Biol 2005; 25:1359-1363), suggesting a potential signaling convergence between the two receptors. This is supported by the observation that CRP can induce endothelial LOX-1 expression through the Fcγ receptors resulting in the induction of endothelial cell-monocyte adhesion and oxLDL uptake (Li L, Roumeliotis N, Sawamura T, Renier G. Circ Res. 2004; 95:877-883). It may be possible that early in inflammation, CRP induces LOX-1 expression through the activation of Fcγ receptors. Once LOX-1 is expressed, it may function synergistically with the Fcγ receptors and lead to exacerbation of endothelial dysfunction.

In conclusion, LOX-1 has been shown to be a novel receptor for CRP. LOX-1 appears to bind CRP via a novel mechanism, which needs to be elucidated through additional studies. In human aortic endothelial cells, the interaction between CRP and LOX-1 elicits pro-inflammatory responses that can be inhibited with an anti-LOX-1 antibody.

### Example 4

### LOX-1 Activation by Oxidized LDL (oxLDL) or C-Reactive Protein (CRP) Treatment Increases Interleukin-8 Expression

### Materials and Methods

Generation of LOX-1 cDNA and Adenovirus

The open reading frame of human LOX-1 (NM_{—}002543) was purchased from Origene (SC118589) and subcloned into pAdori-vector [Kotlyarov, A., et al., Distinct cellular functions of MK2. Mol Cell Biol, 2002. 22(13): p. 4827-35]. Adenovirus expressing LOX-1 and GFP were generated and purified by ViraQuest.

### HAECT Cell Culture and Adenoviral Infection

The HAECT cells are an immortalized human aortic endothelial cell line derived by infection of human aortic endothelial cells freshly isolated from a 53-year-old woman with Adenovirus-SV40 tsA209 and subsequently characterized for the endothelial characteristics (Chadwick, C. C., L. J. Shaw, and R. C. Winneker, Exp Cell Res, 1998. 239(2): p. 423-9). These cells were cultured in EGM-2 basal media (endothelial cell growth medium-2; Cambrex) supplemented with EGM-2 Endothelial Medium SingleQuot Kit containing FBS and growth factors (Cambrex) and were maintained in 5% CO2 environment at 34C to allow the expression of active large T antigen. Oxidized LDL was obtained from Intracel. The adenoviral infection of HAECT cells was carried out at a multiplicity of infection (MOI) of 150.

### TagMan Real-Time Quantitative PCR

Total RNA from HAECT cells was isolated using QIAshredder and RNeasy Mini Kit according to the manufacturer's instructions (Qiagen). CREM, CXCL2, DUSP1, HMOX1, MMP1, DNER and STC1 mRNA levels were measured by TaqMan real-time quantitative PCR using Assay-on-Demand TaqMan reagents (Applied Biosystems). ABI Prism 7000 sequence detection system (PE Applied Biosystems, Foster City, Calif., USA) Threshold cycle (Cₜ) values were obtained and the values were normalized relative to the 18S internal control. TaqMan real-time quantitative PCR was performed in duplicate and the average values were used for quantification. Data analysis was performed as recommended by the manufacturer using the ΔΔCₜ method (PE Applied Biosystems). Data was analyzed using 1-way ANOVA with a Tukey Post hoc test to compare means.

### Gene Expression Profiling

Total RNA from HAECT cells was isolated using QIAshredder and RNeasy Mini Kit according to the manufacturer's instructions (Qiagen). Double-stranded cDNA was synthesized starting with 5 ug of total RNA using the SuperScript System (Invitrogen, Carlsbad, Calif.). The cDNA was purified by filtration through Multiscreen filter plate (Millipore), and transcribed in vitro using T7 RNA polymerase (Epicentre, Madison, Wis.) and biotinylated nucleotides (Perkin-Elmer, Boston, Mass.). Hybridization buffer containing the spike pool reagent was added to each of the fragmented cRNA mixtures and each sample was hybridized to the Human Genome U133 plus 2.0 array (Affymetrix, Santa Clara) at 45° C. for 18 Hrs as recommended by the manufacturer. The human genome array interrogates the expression of over 47,000 transcripts. The hybridized arrays were washed and stained using Affymetrix Fluidics Station 450 and the EukGE-WS2v5_{—}450 protocol according to established protocols. The staining was performed using streptavidin-phycoerythrin conjugate (SAPE; Molecular Probes, Eugene, Oreg.), followed by biotinylated antibody against streptavidin (Vector Laboratories, Burlingame, Calif.), and then SAPE. The arrays were scanned using an Affymetrix Genechip Scanner and cel files were generated with Affymetrix Microarray Suite 5.0 (MAS 5.0) software.

### Microarray Data Analysis:

Signal values were determined by using the Affymetrix GeneChip Operating Software 1.0 (GCOS). All probe sets on all the arrays were normalized to a mean signal intensity value of 100. The default GCOS statistical values and absolute detection calls were used for all subsequent analyses. Filter 1 Selects for probe sets that are robust and detectable. Removes probe sets that are not present in at least 10% of the samples and do not contain an average signal value of 50 or more. Filter 2 Removes poorly expressed genes less stringently than filter 1. To be considered for analysis, a probe set had to have a minimum signal value of 43 and had to be called “Present” in at least one sample per treatment group (data reduction filter).

### Statistical Analysis

ANOVA (analysis of variance) was performed on log-transformed signal values. Multiple factor ANOVA was performed to determine the probe sets significantly regulated by both LOX-1 and treatment with OxLDL with and without a time component. Multiple testing corrections were resolved by applying the BH procedure for controlling FDR to the set of raw p values derived from ANOVA.

### ELISA

Conditioned media was collected and cytokine levels determined by ELISA by Thermo Fisher Scientific—SearchLight Service. Data was analyzed using 1-Way ANOVA with a Tukey post hoc test to compare means *=p<0.05,**=p<0.001.

### Dil-OxLDL Binding Assay

HAECT cells were plated 2×10⁴ cells/well in 96 well plate. The next day the medium was changed to 10% Lipo-Deficient-FBS media. On day three the cells were placed on ice for 30 minutes to bock internalization. 50 ug/ml Dil-OxLDL was added for 2 Hrs while maintaining cells on ice. Cells were washed with PBS three times and examined for Dil-OxLDL binding using microscopy (Nikon Instruments Inc. model TE300) and SPOT imaging software (Diagnostics Instruments). For comparison between experimental conditions images were acquired using identical camera settings.

### Dil-OxLDL Uptake Assay

HAECT cells were plated 1×10⁵ cells/well in 24-well plate. The next day the medium was changed to 10% Lipo-Deficient-FBS media. On day three cells were treated with ug/ml Dil-OxLDL for six Hrs. Cells were washed with PBS three times and examined for Dil-OxLDL uptake using microscopy as described for the Dil-OxLDL binding assay.

### LOX-1 Western

HAECT cells were lysed in Non-denaturing lysis buffer (1% (w/v) Triton X-100, 50 mM TrisCl, pH 7.4, 300 mM NaCl, 5 mM EDTA plus freshly added protease inhibitors). Equal amounts of protein were loaded for SDS-PAGE analysis. Protein gels were transferred to PVDF membranes (Bio Rad). Blots were probed for LOX-1 using goat anti-human LOX-1 (R&D). Proteins were visualized with chemiluminescence using ECL substrate (Amersham Biosciences).

### Results

### Overexpression of LOX-1 in HAECT Cells and the Effect of OxLDL or CRP Treatment

Similarly to endothelial cells under normal physiological conditions, the human aortic endothelial cell line HAECT cells normally express low levels of LOX-1. In order to investigate LOX-1 specific effects in these cells, adenovirus was used to overexpress human LOX-1 in HAECT cells. Infection of these cells with a GFP-expressing adenovirus at MOI of 150 resulted in 90-100% infection (data not shown). The expression of LOX-1 in LOX-1 adenovirus infected cells was confirmed by Western blot analysis (data not shown). The expressed LOX-1 was functional as shown by its ability to bind and uptake Dil fluorescently labeled OxLDL (data not shown).

A cDNA microarray analysis was carried out to evaluate the effects of OxLDL or CRP in LOX-1 expressing HAECT cells and to identify novel gene targets for activated LOX-1. For this purpose, the HAECT cells were infected with LOX-1 or control GFP expressing adenovirus in triplicates, respectively. The GFP-expressing cells are hereafter referred to as control cells. To study the OxLDL or CRP response, LOX-1-expressing or control cells were treated with or without 50 ug/ml OxLDL or 25 ug/ml of CRP for 0, 2, 6, 12, and 24 Hrs. At the end of each treatment, total RNA was isolated for each sample and subjected to gene expression profiling analysis on Affymetrix microarrays. The Affymetrix microarrays did not detect LOX-1 mRNA, likely due to improperly functioning probe sets for this molecule. To confirm the over-expression of LOX-1 in the study, real-time PCR analysis was carried out on the same RNA samples used for transcriptional profiling. The expression of LOX-1 was increased markedly in LOX-1 adenovirus infected cells compared to control cells (data not shown).

### Gene Expression Changes Elicited by OxLDL in HAECT Cells

The Affymetrix microarray HG-U133 2.0 was used for profiling analysis, which interrogated the expression levels of over 47,000 probe sets. For reference, a probe set is defined as a sequence corresponding to a fragment of a specific gene on the microarray. One gene can be represented by different probe sets corresponding to distinct sequences.

OxLDL-induced or CRP-induced transcriptional response was studied. For data analysis, a data reduction filter was first applied to remove lowly expressed genes (Filter 1, described in Materials and Methods), followed by a two-factor ANOVA (infection and treatment factors), after which the effect of OxLDL or CRP treatment was determined. Several genes showed significant changes in this system.

Robust Transcriptional Changes Associated with LOX-1 Expression and OxLDL or CRP Treatment

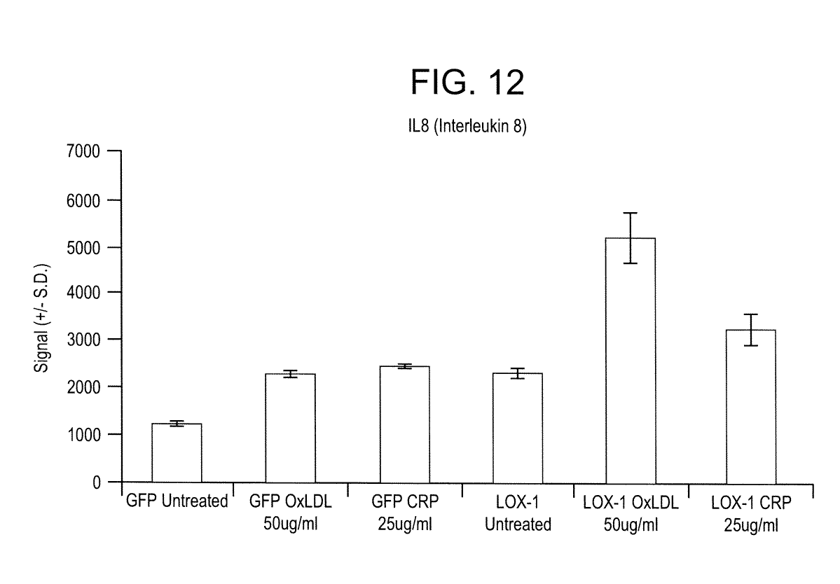

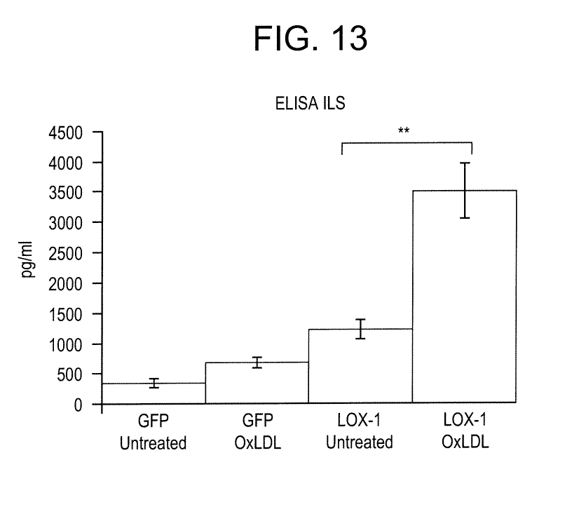

To identify genes with the most robust changes as a function of LOX-1 and OxLDL or CRP treatment, a two-factor ANOVA (infection and treatment Factors) was applied to probe sets that passed Filter 2 (see Methods) at each time point. This led to the identification of probe sets that were dependent on LOX-1 and OxLDL or CRP treatment (p<0.05). To isolate the robustly responding genes, the probe sets were subsequently filtered to select for genes that changed 1.4 fold or greater with OxLDL or CRP treatment in LOX-1 expressing cells. As shown in FIG. 12, treatment of LOX-1 expressing cells with oxLDL or CRP resulted in an increase in Interleukin-8 (IL-8) expression. In order to confirm the microarray data on this gene, Taqman real-time PCR analysis and ELISA were performed and the results confirmed the LOX-1 dependent IL-8 expression changes in response to OxLDL. As shown in FIG. 13, in response to LOX-1 activation by oxLDL, LOX-1 triggers an increase in expression and secretion of IL-8, as shown by ELISA.

TABLE 3
LOX-1 ANTIBODY Sequence Identifiers and Description

| SEQ ID NO | Designation and Description of rat anti-mouse LOX-1 antibodies |
|---|---|
| 1 | LOX1-11A2 V_{L} chain amino acid sequence without signal sequence |
| 2 | LOX1-11A2 V_{L} chain nucleic acid sequence without signal sequence |
| 3 | LOX1-11A2 V_{H} chain amino acid sequence without signal sequence |
| 4 | LOX1-11A2 V_{H} chain nucleic acid sequence without signal sequence |
| 5 | LOX1-33F1 V_{L} chain amino acid sequence without signal sequence |
| 6 | LOX1-33F1 V_{L} chain nucleic acid sequence without signal sequence |
| 7 | LOX1-33F1 V_{H} chain amino acid sequence without signal sequence |
| 8 | LOX1-33F1 V_{H} chain nucleic acid sequence without signal sequence |
| 9 | LOX1-11A2 V_{L} chain amino acid sequence with signal sequence |
| 10 | LOX1-11A2 V_{L} chain nucleic acid sequence with signal sequence |
| 11 | LOX1-11A2 V_{H} chain amino acid sequence with signal sequence |
| 12 | LOX1-11A2 V_{H} chain nucleic acid sequence with signal sequence |
| 13 | LOX1-33F1 V_{L} chain amino acid sequence with signal sequence |
| 14 | LOX1-33F1 V_{L} chain nucleic acid sequence with signal sequence |
| 15 | LOX1-33F1 V_{H} chain amino acid sequence with signal sequence |
| 16 | LOX1-33F1 V_{H} chain nucleic acid sequence with signal sequence |

What is claimed is:

1. A method of selectively inhibiting binding of a ligand to a lectin-like oxidized low-density lipoprotein receptor (LOX-1), comprising contacting the LOX-1 with a binding partner that inhibits binding of at least one ligand to LOX-1, but not one other ligand for LOX-1.

2. The method of claim 1, wherein the LOX-1 is on a cell selected from the group consisting of an endothelial cell, a macrophage, a monocyte, a dendritic cell, a vascular smooth muscle cell (SMC), a chondrocyte, a platelet, an intestinal cell, and a cardiac myocyte.

3. The method of claim 1, wherein the binding partner is an antibody.

4. The method of claim 1, wherein the ligands comprise oxidized low density lipoprotein (ox-LDL) and C reactive protein (CRP).

5. The method of claim 1, wherein the binding partner inhibits the binding of oxidized LDL with LOX-1, but does not inhibit the binding of C reactive protein with LOX-1.

6. The method of claim 3, wherein the antibody is a rat anti-LOX-1 antibody.

7. The method of claim 6, wherein the antibody comprises a variable light (V_{L}) chain comprising the amino acid sequence of SEQ ID NO: 1 or SEQ ID NO: 5.

8. The method of claim 6, wherein the antibody comprises a variable heavy (V_{H}) chain comprising the amino acid sequence of SEQ ID NO: 3 or SEQ ID NO: 7.

9. The method of claim 6, wherein the antibody comprises a variable light (V_{L}) chain comprising the amino acid sequence of SEQ ID NO: 1 and a variable heavy (V_{H}) chain comprising the amino acid sequence of SEQ ID NO: 3.

10. The method of claim 9, wherein the V_{L} chain is encoded by a nucleic acid sequence comprising the nucleic acid sequence of SEQ ID NO: 2 and wherein the V_{H} chain is encoded by a nucleic acid sequence comprising the nucleic acid sequence of SEQ ID NO: 4.

11. The method of claim 6, wherein the antibody comprises a variable light (V_{L}) chain comprising the amino acid sequence of SEQ ID NO: 5 and a variable heavy (V_{H}) chain comprising the amino acid sequence of SEQ ID NO: 7.

12. The method of claim 6, wherein the V_{L} chain is encoded by a nucleic acid sequence comprising the nucleic acid sequence of SEQ ID NO: 6 and wherein the V_{H} chain is encoded by a nucleic acid sequence comprising the nucleic acid sequence of SEQ ID NO: 8.

13. The method of claim 1, wherein the ligand that binds to LOX-1 is selected from the group consisting of a modified lipoprotein, an anionic phospholipid, a cellular ligand, a bile salt-dependent lipase and C-reactive protein.

14. The method of claim 13, wherein the modified lipoprotein is selected from the group consisting of oxidized low density lipoprotein (ox-LDL), acetylated low density lipoprotein (Ac-LDL), and advanced glycation end-products (AGEs).

15. The method of claim 13, wherein the anionic phospholipids is phosphatidylserine or phosphatidylinositol.

16. The method of claim 13, wherein the cellular ligand is selected from the group consisting of apoptotic cells, aged cells, activated platelets and bacterial cells.

17. The method of claim 1, wherein the method results in elimination of at least one detrimental biological effect associated with ligand binding to LOX-1, but retains one or more other non-detrimental biological effects associated with ligand binding to LOX-1, wherein the at least one detrimental effect associated with ligand binding to LOX-1 is endothelial cell dysfunction.

18. An isolated or purified binding partner that interacts with, or binds to LOX-1, wherein the binding partner is characterized by its ability to inhibit the binding of at least one ligand to LOX-1, but not one other ligand for LOX-1.

19. The binding partner of claim 18, wherein the binding partner is an antibody.

20. The antibody of claim 6, wherein the antibody is a rat anti-LOX-1 antibody.

21. The antibody of claim 20, wherein the antibody comprises a variable light (V_{L}) chain comprising the amino acid sequence of SEQ ID NO: 1 or SEQ ID NO: 5.

22. The antibody of claim 20, wherein the antibody comprises a variable heavy (V_{H}) chain comprising the amino acid sequence of SEQ ID NO: 3 or SEQ ID NO: 7.

23. The antibody of claim 20, wherein the antibody comprises a variable light (V_{L}) chain comprising the amino acid sequence of SEQ ID NO: 1 and a variable heavy (V_{H}) chain comprising the amino acid sequence of SEQ ID NO: 3.

24. The antibody of claim 20, wherein the antibody comprises a variable light (V_{L}) chain comprising the amino acid sequence of SEQ ID NO: 5 and a variable heavy (V_{H}) chain comprising the amino acid sequence of SEQ ID NO: 7.

25. The antibody of claim 24, wherein the V_{L} chain is encoded by a nucleic acid sequence comprising the nucleic acid sequence of SEQ ID NO: 6 and wherein the V_{H} chain is encoded by a nucleic acid sequence comprising the nucleic acid sequence of SEQ ID NO: 8.

26. The binding partner of claim 18, wherein the binding partner inhibits the binding of oxidized LDL with LOX-1, but does not inhibit the binding of C reactive protein with LOX-1.

27. A pharmaceutical composition comprising a therapeutically effective amount of the binding partner of claim 18.

28. A method of treating a mammal suffering from a disease or condition associated with elevated levels of LOX-1 or a LOX-1 ligand, comprising administering an isolated or purified LOX-1 binding partner of claim 18 and a pharmaceutically acceptable carrier.

29. The method of claim 28, wherein the mammal is human.

30. The method of claim 28, wherein the disease or condition is selected from the group consisting of atherosclerosis, hypertension, hyperlipidemia, hypercholesterolemia, diabetes mellitus, nitric oxide deficiency, osteoarthritis, myocardial infarction, ischemia-reperfusion, sepsis, diabetic nephropathy, renal disease, cardiomyopathy, heart failure, peripheral artery disease, coronary heart disease and tumor cell proliferation.
